SEQUENCE LISTING (16 sequences)

SEQ ID NO: 1 - protein, 2347 aa, Homo sapiens
MKNIYCLIPKLVNFATLGCLWISVVQCTVLNSCLKSCVTNLGQQLDLGTPHNLSEPCIQGCHFWNSVDQKNCALKCRESCEVGCSSAEGAYEEEVLENADLPTAPFASSIGSHNMTLRWKSANFSGVKYIIQWKYAQLLGSWTYTKTVSRPSYVVKPLHPFTEYIFRVVWIFTAQLQLYSPPSPSYRTHPHGVPETAPLIRNIESSSPDTVEVSWDPPQFPGGPILGYNLRLISKNQKLDAGTQRTSFQFYSTLPNTIYRFSIAAVNEVGEGPEAESSITTSSSAVQQEEQWLFLSRKTSLRKRSLKHLVDEAHCLRLDAIYHNITGISVDVHQQIVYFSEGTLIWAKKAANMSDVSDLRIFYRGSGLISSISIDWLYQRMYFIMDELVCVCDLENCSNIEEITPPSISAPQKIVADSYNGYVFYLLRDGIYRADLPVPSGRCAEAVRIVESCTLKDFAIKPQAKRIIYFNDTAQVFMSTFLDGSASHLILPRIPFADVKSFACENNDFLVTDGKVIFQQDALSFNEFIVGCDLSHIEEFGFGNLVIFGSSSQLHPLPGRPQELSVLFGSHQALVQWKPPALAIGANVILISDIIELFELGPSAWQNWTYEVKVSTQDPPEVTHIFLNISGTMLNVPELQSAMKYKVSVRASSPKRPGPWSEPSVGTTLVPASEPPFIMAVKEDGLWSKPLNSFGPGEFLSSDIGNVSDMDWYNNSLYYSDTKGDVFVWLLNGTDISENYHLPSIAGAGALAFEWLGHFLYWAGKTYVIQRQSVLTGHTDIVTHVKLLVNDMVVDSVGGYLYWTTLYSVESTRLNGESSLVLQTQPWFSGKKVIALTLDLSDGLLYWLVQDSQCIHLYTAVLRGQSTGDTTITEFAAWSTSEISQNALMYYSGRLFWINGFRIITTQEIGQKTSVSVLEPARFNQFTIIQTSLKPLPGNFSFTPKVIPDSVQESSFRIEGNASSFQILWNGPPAVDWGVVFYSVEFSAHSKFLASEQHSLPVFTVEGLEPYALFNLSVTPYTYWGKGPKTSLSLRAPETVPSAPENPRIFILPSGKCCNKNEVVVEFRWNKPKHENGVLTKFEIFYNISNQSITNKTCEDWIAVNVTPSVMSFQLEGMSPRCFIAFQVRAFTSKGPGPYADVVKSTTSEINPFPHLITLLGNKIVFLDMDQNQVVWTFSAERVISAVCYTADNEMGYYAEGDSLFLLHLHNRSSSELFQDSLVFDITVITIDWISRHLYFALKESQNGMQVFDVDLEHKVKYPREVKIHNRNSTIISFSVYPLLSRLYWTEVSNFGYQMFYYSIISHTLHRILQPTATNQQNKRNQCSCNVTEFELSGAMAIDTSNLEKPLIYFAKAQEIWAMDLEGCQCWRVITVPAMLAGKTLVSLTVDGDLIYWIITAKDSTQIYQAKKGNGAIVSQVKALRSRHILAYSSVMQPFPDKAFLSLASDTVEPTILNATNTSLTIRLPLAKTNLTWYGITSPTPTYLVYYAEVNDRKNSSDLKYRILEFQDSIALIEDLQPFSTYMIQIAVKNYYSDPLEHLPPGKEIWGKTKNGVPEAVQLINTTVRSDTSLIISWRESHKPNGPKESVRYQLAISHLALIPETPLRQSEFPNGRLTLLVTRLSGGNIYVLKVLACHSEEMWCTESHPVTVEMFNTPEKPYSLVPENTSLQFNWKAPLNVNLIRFWVELQKWKYNEFYHVKTSCSQGPAYVCNITNLQPYTSYNVRVVVVYKTGENSTSLPESFKTKAGVPNKPGIPKLLEGSKNSIQWEKAEDNGCRITYYILEIRKSTSNNLQNQNLRWKMTFNGSCSSVCTWKSKNLKGIFQFRVVAANNLGFGEYSGISENIILVGDDFWIPETSFILTIIVGIFLVVTIPLTFVWHRRLKNQKSAKEGVTVLINEDKELAELRGLAAGVGLANACYAIHTLPTQEEIENLPAFPREKLTLRLLLGSGAFGEVYEGTAVDILGVGSGEIKVAVKTLKKGSTDQEKIEFLKEAHLMSKFNHPNILKQLGVCLLNEPQYIILELMEGGDLLTYLRKARMATFYGPLLTLVDLVDLCVDISKGCVYLERMHFIHRDLAARNCLVSVKDYTSPRIVKIGDFGLARDIYKNDYYRKRGEGLLPVRWMAPESLMDGIFTTQSDVWSFGILIWEILTLGHQPYPAHSNLDVLNYVQTGGRLEPPRNCPDDLWNLMTQCWAQEPDQRPTFHRIQDQLQLFRNFFLNSIYKSRDEANNSGVINESFEGEDGDVICLNSDDIMPVALMETKNREGLNYMVLATECGQGEEKSEGPLGSQESESCGLRKEEKEPHADKDFCQEKQVAYCPSGKPEGLNYACLTHSGYGDGSD

SEQ ID NO: 2 - protein, 1114 aa, Homo sapiens
MAKATSGAAGLRLLLLLLLPLLGKVALGLYFSRDAYWEKLYVDQAAGTPLLYVHALRDAPEEVPSFRLGQHLYGTYRTRLHENNWICIQEDTGLLYLNRSLDHSSWEKLSVRNRGFPLLTVYLKVFLSPTSLREGECQWPGCARVYFSFFNTSFPACSSLKPRELCFPETRPSFRIRENRPPGTFHQFRLLPVQFLCPNISVAYRLLEGEGLPFRCAPDSLEVSTRWALDREQREKYELVAVCTVHAGAREEVVMVPFPVTVYDEDDSAPTFPAGVDTASAVVEFKRKEDTVVATLRVFDADVVPASGELVRRYTSTLLPGDTWAQQTFRVEHWPNETSVQANGSFVRATVHDYRLVLNRNLSISENRTMQLAVLVNDSDFQGPGAGVLLLHFNVSVLPVSLHLPSTYSLSVSRRARRFAQIGKVCVENCQAFSGINVQYKLHSSGANCSTLGVVTSAEDTSGILFVNDTKALRRPKCAELHYMVVATDQQTSRQAQAQLLVTVEGSYVAEEAGCPLSCAVSKRRLECEECGGLGSPTGRCEWRQGDGKGITRNFSTCSPSTKTCPDGHCDVVETQDINICPQDCLRGSIVGGHEPGEPRGIKAGYGTCNCFPEEEKCFCEPEDIQDPLCDELCRTVIAAAVLFSFIVSVLLSAFCIHCYHKFAHKPPISSAEMTFRRPAQAFPVSYSSSGARRPSLDSMENQVSVDAFKILEDPKWEFPRKNLVLGKTLGEGEFGKVVKATAFHLKGRAGYTTVAVKMLKENASPSELRDLLSEFNVLKQVNHPHVIKLYGACSQDGPLLLIVEYAKYGSLRGFLRESRKVGPGYLGSGGSRNSSSLDHPDERALTMGDLISFAWQISQGMQYLAEMKLVHRDLAARNILVAEGRKMKISDFGLSRDVYEEDSYVKRSQGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKRRDYLDLAASTPSDSLIYDDGLSEEETPLVDCNNAPLPRALPSTWIENKLYGMSDPNWPGESPVPLTRADGTNTGFPRYPNDSVYANWMLSPSAAKLMDTFDS

SEQ ID NO: 3 - protein, 1620 aa, Homo sapiens
MGAIGLLWLLPLLLSTAAVGSGMGTGQRAGSPAAGPPLQPREPLSYSRLQRKSLAVDFVVPSLFRVYARDLLLPPSSSELKAGRPEARGSLALDCAPLLRLLGPAPGVSWTAGSPAPAEARTLSRVLKGGSVRKLRRAKQLVLELGEEAILEGCVGPPGEAAVGLLQFNLSELFSWWIRQGEGRLRIRLMPEKKASEVGREGRLSAAIRASQPRLLFQIFGTGHSSLESPTNMPSPSPDYFTWNLTWIMKDSFPFLSHRSRYGLECSFDFPCELEYSPPLHDLRNQSWSWRRIPSEEASQMDLLDGPGAERSKEMPRGSFLLLNTSADSKHTILSPWMRSSSEHCTLAVSVHRHLQPSGRYIAQLLPHNEAAREILLMPTPGKHGWTVLQGRIGRPDNPFRVALEYISSGNRSLSAVDFFALKNCSEGTSPGSKMALQSSFTCWNGTVLQLGQACDFHQDCAQGEDESQMCRKLPVGFYCNFEDGFCGWTQGTLSPHTPQWQVRTLKDARFQDHQDHALLLSTTDVPASESATVTSATFPAPIKSSPCELRMSWLIRGVLRGNVSLVLVENKTGKEQGRMVWHVAAYEGLSLWQWMVLPLLDVSDRFWLQMVAWWGQGSRAIVAFDNISISLDCYLTISGEDKILQNTAPKSRNLFERNPNKELKPGENSPRQTPIFDPTVHWLFTTCGASGPHGPTQAQCNNAYQNSNLSVEVGSEGPLKGIQIWKVPATDTYSISGYGAAGGKGGKNTMMRSHGVSVLGIFNLEKDDMLYILVGQQGEDACPSTNQLIQKVCIGENNVIEEEIRVNRSVHEWAGGGGGGGGATYVFKMKDGVPVPLIIAAGGGGRAYGAKTDTFHPERLENNSSVLGLNGNSGAAGGGGGWNDNTSLLWAGKSLQEGATGGHSCPQAMKKWGWETRGGFGGGGGGCSSGGGGGGYIGGNAASNNDPEMDGEDGVSFISPLGILYTPALKVMEGHGEVNIKHYLNCSHCEVDECHMDPESHKVICFCDHGTVLAEDGVSCIVSPTPEPHLPLSLILSVVTSALVAALVLAFSGIMIVYRRKHQELQAMQMELQSPEYKLSKLRTSTIMTDYNPNYCFAGKTSSISDLKEVPRKNITLIRGLGHGAFGEVYEGQVSGMPNDPSPLQVAVKTLPEVCSEQDELDFLMEALIISKFNHQNIVRCIGVSLQSLPRFILLELMAGGDLKSFLRETRPRPSQPSSLAMLDLLHVARDIACGCQYLEENHFIHRDIAARNCLLTCPGPGRVAKIGDFGMARDIYRASYYRKGGCAMLPVKWMPPEAFMEGIFTSKTDTWSFGVLLWEIFSLGYMPYPSKSNQEVLEFVTSGGRMDPPKNCPGPVYRIMTQCWQHQPEDRPNFAIILERIEYCTQDPDVINTALPIEYGPLVEEEEKVPVRPKDPEGVPPLLVSQQAKREEERSPAAPPPLPTTSSGKAAKKPTAAEISVRVPRGPAVEGGHVNMAFSQSNPPSELHKVHGSRNKPTSLWNPTYGSWFTEKPTKKNNPIAKKEPHDRGNLGLEGSCTVPPNVATGRLPGASLLLEPSSLTANMKEVPLFRLRHFPCGNVNYGYQQQGLPLEAATAPGAGHYEDTILKSKNSMNQPGP

SEQ ID NO: 4 - protein, 760 aa, Homo sapiens
MKEAALICLAPSVPPILTVKSWDTMQLRAARSRCTNLLAASYIENQQHLQHLELRDLRGLGELRNLTIVKSGLRFVAPDAFHFTPRLSRLNLSFNALESLSWKTVQGLSLQELVLSGNPLHCSCALRWLQRWEEEGLGGVPEQKLQCHGQGPLAHMPNASCGVPTLKVQVPNASVDVGDDVLLRCQVEGRGLEQAGWILTELEQSATVMKSGGLPSLGLTLANVTSDLNRKNVTCWAENDVGRAEVSVQVNVSFPASVQLHTAVEMHHWCIPFSVDGQPAPSLRWLFNGSVLNETSFIFTEFLEPAANETVRHGCLRLNQPTHVNNGNYTLLAANPFGQASASIMAAFMDNPFEFNPEDPIPDTNSTSGDPVEKKDETPFGVSVAVGLAVFACLFLSTLLLVLNKCGRRNKFGINRPAVLAPEDGLAMSLHFMTLGGSSLSPTEGKGSGLQGHIIENPQYFSDACVHHIKRRDIVLKWELGEGAFGKVFLAECHNLLPEQDKMLVAVKALKEASESARQDFQREAELLTMLQHQHIVRFFGVCTEGRPLLMVFEYMRHGDLNRFLRSHGPDAKLLAGGEDVAPGPLGLGQLLAVASQVAAGMVYLAGLHFVHRDLATRNCLVGQGLVVKIGDFGMSRDIYSTDYYRVGGRTMLPIRWMPPESILYRKFTTESDVWSFGVVLWEIFTYGKQPWYQLSNTEAIDCITQGRELERPRACPPEVYAIMRGCWQREPQQRHSIKDVHARLQALAQAPPVYLDVLG

SEQ ID NO: 5 - protein, 838 aa, Homo sapiens
MSSWIRWHGPAMARLWGFCWLVVGFWRAAFACPTSCKCSASRIWCSDPSPGIVAFPRLEPNSVDPENITEIFIANQKRLEIINEDDVEAYVGLRNLTIVDSGLKFVAHKAFLKNSNLQHINFTRNKLTSLSRKHFRHLDLSELILVGNPFTCSCDIMWIKTLQEAKSSPDTQDLYCLNESSKNIPLANLQIPNCGLPSANLAAPNLTVEEGKSITLSCSVAGDPVPNMYWDVGNLVSKHMNETSHTQGSLRITNISSDDSGKQISCVAENLVGEDQDSVNLTVHFAPTITFLESPTSDHHWCIPFTVKGNPKPALQWFYNGAILNESKYICTKIHVTNHTEYHGCLQLDNPTHMNNGDYTLIAKNEYGKDEKQISAHFMGWPGIDDGANPNYPDVIYEDYGTAANDIGDTTNRSNEIPSTDVTDKTGREHLSVYAVVVIASVVGFCLLVMLFLLKLARHSKFGMKDFSWFGFGKVKSRQGVGPASVISNDDDSASPLHHISNGSNTPSSSEGGPDAVIIGMTKIPVIENPQYFGITNSQLKPDTFVQHIKRHNIVLKRELGEGAFGKVFLAECYNLCPEQDKILVAVKTLKDASDNARKDFHREAELLTNLQHEHIVKFYGVCVEGDPLIMVFEYMKHGDLNKFLRAHGPDAVLMAEGNPPTELTQSQMLHIAQQIAAGMVYLASQHFVHRDLATRNCLVGENLLVKIGDFGMSRDVYSTDYYRVGGHTMLPIRWMPPESIMYRKFTTESDVWSLGVVLWEIFTYGKQPWYQLSNNEVIECITQGRVLQRPRTCPQEVYELMLGCWQREPHMRKNIKGIHTLLQNLAKASPVYLDILG

SEQ ID NO: 6 - protein, 839 aa, Homo sapiens
MDVSLCPAKCSFWRIFLLGSVWLDYVGSVLACPANCVCSKTEINCRRPDDGNLFPLLEGQDSGNSNGNASINITDISRNITSIHIENWRSLHTLNAVDMELYTGLQKLTIKNSGLRSIQPRAFAKNPHLRYINLSSNRLTTLSWQLFQTLSLRELQLEQNFFNCSCDIRWMQLWQEQGEAKLNSQNLYCINADGSQLPLFRMNISQCDLPEISVSHVNLTVREGDNAVITCNGSGSPLPDVDWIVTGLQSINTHQTNLNWTNVHAINLTLVNVTSEDNGFTLTCIAENVVGMSNASVALTVYYPPRVVSLEEPELRLEHCIEFVVRGNPPPTLHWLHNGQPLRESKIIHVEYYQEGEISEGCLLFNKPTHYNNGNYTLIAKNPLGTANQTINGHFLKEPFPESTDNFILFDEVSPTPPITVTHKPEEDTFGVSIAVGLAAFACVLLVVLFVMINKYGRRSKFGMKGPVAVISGEEDSASPLHHINHGITTPSSLDAGPDTVVIGMTRIPVIENPQYFRQGHNCHKPDTYVQHIKRRDIVLKRELGEGAFGKVFLAECYNLSPTKDKMLVAVKALKDPTLAARKDFQREAELLTNLQHEHIVKFYGVCGDGDPLIMVFEYMKHGDLNKFLRAHGPDAMILVDGQPRQAKGELGLSQMLHIASQIASGMVYLASQHFVHRDLATRNCLVGANLLVKIGDFGMSRDVYSTDYYRLFNPSGNDFCIWCEVGGHTMLPIRWMPPESIMYRKFTTESDVWSFGVILWEIFTYGKQPWFQLSNTEVIECITQGRVLERPRVCPKEVYDVMLGCWQREPQQRLNIKEIYKILHALGKATPIYLDILG

SEQ ID NO: 7 - protein, 648 aa, Homo sapiens [sequence omitted]

SEQ ID NO: 8 - protein, 766 aa, Homo sapiens [sequence omitted]

SEQ ID NO: 9 - protein, 672 aa, Homo sapiens
MADVFPGNDSTASQDVANRFARKGALRQKNVHEVKDHKFIARFFKQPTFCSHCTDFIWGFGKQGFQCQVCCFVVHKRCHEFVTFSCPGADKGPDTDDPRSKHKFKIHTYGSPTFCDHCGSLLYGLIHQGMKCDTCDMNVHKQCVINVPSLCGMDHTEKRGRIYLKAEVADEKLHVTVRDAKNLIPMDPNGLSDPYVKLKLIPDPKNESKQKTKTIRSTLNPQWNESFTFKLKPSDKDRRLSVEIWDWDRTTRNDFMGSLSFGVSELMKMPASGWYKLLNQEEGEYYNVPIPEGDEEGNMELRQKFEKAKLGPAGNKVISPSEDRKQPSNNLDRVKLTDFNFLMVLGKGSFGKVMLADRKGTEELYAIKILKKDVVIQDDDVECTMVEKRVLALLDKPPFLTQLHSCFQTVDRLYFVMEYVNGGDLMYHIQQVGKFKEPQAVFYAAEISIGLFFLHKRGIIYRDLKLDNVMLDSEGHIKIADFGMCKEHMMDGVTTRTFCGTPDYIAPEIIAYQPYGKSVDWWAYGVLLYEMLAGQPPFDGEDEDELFQSIMEHNVSYPKSLSKEAVSVCKGLMTKHPAKRLGCGPEGERDVREHAFFRRIDWEKLENREIQPPFKPKVCGKGAENFDKFFTRGQPVLTPPDQLVIANIDQSDFEGFSYVNPQFVHPILQSAV

SEQ ID NO: 10 - protein, 671 aa, Homo sapiens
MADPAAGPPPSEGEESTVRFARKGALRQKNVHEVKNHKFTARFFKQPTFCSHCTDFIWGFGKQGFQCQVCCFVVHKRCHEFVTFSCPGADKGPASDDPRSKHKFKIHTYSSPTFCDHCGSLLYGLIHQGMKCDTCMMNVHKRCVMNVPSLCGTDHTERRGRIYIQAHIDRDVLIVLVRDAKNLVPMDPNGLSDPYVKLKLIPDPKSESKQKTKTIKCSLNPEWNETFRFQLKESDKDRRLSVEIWDWDLTSRNDFMGSLSFGISELQKASVDGWFKLLSQEEGEYFNVPVPPEGSEANEELRQKFERAKISQGTKVPEEKTTNTVSKFDNNGNRDRMKLTDFNFLMVLGKGSFGKVMLSERKGTDELYAVKILKKDVVIQDDDVECTMVEKRVLALPGKPPFLTQLHSCFQTMDRLYFVMEYVNGGDLMYHIQQVGRFKEPHAVFYAAEIAIGLFFLQSKGIIYRDLKLDNVMLDSEGHIKIADFGMCKENIWDGVTTKTFCGTPDYIAPEIIAYQPYGKSVDWWAFGVLLYEMLAGQAPFEGEDEDELFQSIMEHNVAYPKSMSKEAVAICKGLMTKHPGKRLGCGPEGERDIKEHAFFRYIDWEKLERKEIQPPYKPKARDKRDTSNFDKEFTRQPVELTPTDKLFIMNLDQNEFAGFSYTNPEFVINV

SEQ ID NO: 11 - protein, 942 aa, Homo sapiens
MASDAVQSEPRSWSLLEQLGLAGADLAAPGVQQQLELERERLRREIRKELKLKEGAENLRRATTDLGRSLGPVELLLRGSSRRLDLLHQQLQELHAHVVLPDPAATHDGPQSPGAGGPTCSATNLSRVAGLEKQLAIELKVKQGAENMIQTYSNGSTKDRKLLLTAQQMLQDSKTKIDIIRMQLRRALQAGQLENQAAPDDTQGSPDLGAVELRIEELRHHFRVEHAVAEGAKNVLRLLSAAKAPDRKAVSEAQEKLTESNQKLGLLREALERRLGELPADHPKGRLLREELAAASSAAFSTRLAGPFPATHYSTLCKPAPLTGTLEVRVVGCRDLPETIPWNPTPSMGGPGTPDSRPPFLSRPARGLYSRSGSLSGRSSLKAEAENTSEVSTVLKLDNTVVGQTSWKPCGPNAWDQSFTLELERARELELAVFWRDQRGLCALKFLKLEDFLDNERHEVQLDMEPQGCLVAEVTFRNPVIERIPRLRRQKKIFSKQQGKAFQRARQMNIDVATWVRLLRRLIPNATGTGTFSPGASPGSEARTTGDISVEKLNLGTDSDSSPQKSSRDPPSSPSSLSSPIQESTAPELPSETQETPGPALCSPLRKSPLTLEDFKFLAVLGRGHFGKVLLSEFRPSGELFAIKALKKGDIVARDEVESLMCEKRILAAVTSAGHPFLVNLFGCFQTPEHVCFVMEYSAGGDLMLHIHSDVFSEPRAIFYSACVVLGLQFLHEHKIVYRDLKLDNLLLDTEGYVKIADFGLCKEGMGYGDRTSTFCGTPEFLAPEVLTDTSYTRAVDWWGLGVLLYEMLVGESPFPGDDEEEVFDSIVNDEVRYPRFLSAEAIGIMRRLLRRNPERRLGSSERDAEDVKKQPFFRTLGWEALLARRLPPPFVPTLSGRTDVSNFDEEFTGEAPTLSPPRDARPLTAAEQAAFLDFDFVAGGC

SEQ ID NO: 12 - protein, 790 aa, Homo sapiens
MLRGGRRGQLGWHSWAAGPGSLLAWLILASAGAAPCPDACCPHGSSGLRCTRDGALDSLHHLPGAENLTELYIENQQHLQHLELRDLRGLGELRNLTIVKSGLRFVAPDAFHFTPRLSRLNLSFNALESLSWKTVQGLSLQELVLSGNPLHCSCALRWLQRWEEEGLGGVPEQKLQCHGQGPLAHMPNASCGVPTLKVQVPNASVDVGDDVLLRCQVEGRGLEQAGWILTELEQSATVMKSGGLPSLGLTLANVTSDLNRKNVTCWAENDVGRAEVSVQVNVSFPASVQLHTAVEMHHWCIPFSVDGQPAPSLRWLFNGSVLNETSFIFTEFLEPAANETVRHGCLRLNQPTHVNNGNYTLLAANPFGQASASIMAAFMDNPFEFNPEDPIPDTNSTSGDPVEKKDETPFGVSVAVGLAVFACLFLSTLLLVLNKCGRRNKFGINRPAVLAPEDGLAMSLHFMTLGGSSLSPTEGKGSGLQGHIIENPQYFSDACVHHIKRRDIVLKWELGEGAFGKVFLAECHNLLPEQDKMLVAVKALKEASESARQDFQREAELLTMLQHQHIVRFFGVCTEGRPLLMVFEYMRHGDLNRFLRSHGPDAKLLAGGEDVAPGPLGLGQLLAVASQVAAGMVYLAGLHFVHRDLATRNCLVGQGLVVKIGDFGMSRDIYSTDYYRVGGRTMLPIRWMPPESILYRKFTTESDVWSFGVVLWEIFTYGKQPWYQLSNTEAIDCITQGRELERPRACPPEVYAIMRGCWQREPQQRHSIKDVHARLQALAQAPPVYLDVLG

SEQ ID NO: 13 - protein, 796 aa, Homo sapiens
MLRGGRRGQLGWHSWAAGPGSLLAWLILASAGAAPCPDACCPHGSSGLRCTRDGALDSLHHLPGAENLTELYIENQQHLQHLELRDLRGLGELRNLTIVKSGLRFVAPDAFHFTPRLSRLNLSFNALESLSWKTVQGLSLQELVLSGNPLHCSCALRWLQRWEEEGLGGVPEQKLQCHGQGPLAHMPNASCGVPTLKVQVPNASVDVGDDVLLRCQVEGRGLEQAGWILTELEQSATVMKSGGLPSLGLTLANVTSDLNRKNVTCWAENDVGRAEVSVQVNVSFPASVQLHTAVEMHHWCIPFSVDGQPAPSLRWLFNGSVLNETSFIFTEFLEPAANETVRHGCLRLNQPTHVNNGNYTLLAANPFGQASASIMAAFMDNPFEFNPEDPIPVSFSPVDTNSTSGDPVEKKDETPFGVSVAVGLAVFACLFLSTLLLVLNKCGRRNKFGINRPAVLAPEDGLAMSLHFMTLGGSSLSPTEGKGSGLQGHIIENPQYFSDACVHHIKRRDIVLKWELGEGAFGKVFLAECHNLLPEQDKMLVAVKALKEASESARQDFQREAELLTMLQHQHIVRFFGVCTEGRPLLMVFEYMRHGDLNRFLRSHGPDAKLLAGGEDVAPGPLGLGQLLAVASQVAAGMVYLAGLHFVHRDLATRNCLVGQGLVVKIGDFGMSRDIYSTDYYRVGGRTMLPIRWMPPESILYRKFTTESDVWSFGVVLWEIFTYGKQPWYQLSNTEAIDCITQGRELERPRACPPEVYAIMRGCWQREPQQRHSIKDVHARLQALAQAPPVYLDVLG

SEQ ID NO: 14 - protein, 822 aa, Homo sapiens
MSSWIRWHGPAMARLWGFCWLVVGFWRAAFACPTSCKCSASRIWCSDPSPGIVAFPRLEPNSVDPENITEIFIANQKRLEIINEDDVEAYVGLRNLTIVDSGLKFVAHKAFLKNSNLQHINFTRNKLTSLSRKHFRHLDLSELILVGNPFTCSCDIMWIKTLQEAKSSPDTQDLYCLNESSKNIPLANLQIPNCGLPSANLAAPNLTVEEGKSITLSCSVAGDPVPNMYWDVGNLVSKHMNETSHTQGSLRITNISSDDSGKQISCVAENLVGEDQDSVNLTVHFAPTITFLESPTSDHHWCIPFTVKGNPKPALQWFYNGAILNESKYICTKIHVTNHTEYHGCLQLDNPTHMNNGDYTLIAKNEYGKDEKQISAHFMGWPGIDDGANPNYPDVIYEDYGTAANDIGDTTNRSNEIPSTDVTDKTGREHLSVYAVVVIASVVGFCLLVMLFLLKLARHSKFGMKGPASVISNDDDSASPLHHISNGSNTPSSSEGGPDAVIIGMTKIPVIENPQYFGITNSQLKPDTFVQHIKRHNIVLKRELGEGAFGKVFLAECYNLCPEQDKILVAVKTLKDASDNARKDFHREAELLTNLQHEHIVKFYGVCVEGDPLIMVFEYMKHGDLNKFLRAHGPDAVLMAEGNPPTELTQSQMLHIAQQIAAGMVYLASQHFVHRDLATRNCLVGENLLVKIGDFGMSRDVYSTDYYRVGGHTMLPIRWMPPESIMYRKFTTESDVWSLGVVLWEIFTYGKQPWYQLSNNEVIECITQGRVLQRPRTCPQEVYELMLGCWQREPHMRKNIKGIHTLLQNLAKASPVYLDILG

SEQ ID NO: 15 - protein, 825 aa, Homo sapiens
MDVSLCPAKCSFWRIFLLGSVWLDYVGSVLACPANCVCSKTEINCRRPDDGNLFPLLEGQDSGNSNGNASINITDISRNITSIHIENWRSLHTLNAVDMELYTGLQKLTIKNSGLRSIQPRAFAKNPHLRYINLSSNRLTTLSWQLFQTLSLRELQLEQNFFNCSCDIRWMQLWQEQGEAKLNSQNLYCINADGSQLPLFRMNISQCDLPEISVSHVNLTVREGDNAVITCNGSGSPLPDVDWIVTGLQSINTHQTNLNWTNVHAINLTLVNVTSEDNGFTLTCIAENVVGMSNASVALTVYYPPRVVSLEEPELRLEHCIEFVVRGNPPPTLHWLHNGQPLRESKIIHVEYYQEGEISEGCLLFNKPTHYNNGNYTLIAKNPLGTANQTINGHFLKEPFPESTDNFILFDEVSPTPPITVTHKPEEDTFGVSIAVGLAAFACVLLVVLFVMINKYGRRSKFGMKGPVAVISGEEDSASPLHHINHGITTPSSLDAGPDTVVIGMTRIPVIENPQYFRQGHNCHKPDTYVQHIKRRDIVLKRELGEGAFGKVFLAECYNLSPTKDKMLVAVKALKDPTLAARKDFQREAELLTNLQHEHIVKFYGVCGDGDPLIMVFEYMKHGDLNKFLRAHGPDAMILVDGQPRQAKGELGLSQMLHIASQIASGMVYLASQHFVHRDLATRNCLVGANLLVKIGDFGMSRDVYSTDYYRVGGHTMLPIRWMPPESIMYRKFTTESDVWSFGVILWEIFTYGKQPWFQLSNTEVIECITQGRVLERPRVCPKEVYDVMLGCWQREPQQRLNIKEIYKILHALGKATPIYLDILG

SEQ ID NO: 16 - protein, 831 aa, Homo sapiens
MDVSLCPAKCSFWRIFLLGSVWLDYVGSVLACPANCVCSKTEINCRRPDDGNLFPLLEGQDSGNSNGNASINITDISRNITSIHIENWRSLHTLNAVDMELYTGLQKLTIKNSGLRSIQPRAFAKNPHLRYINLSSNRLTTLSWQLFQTLSLRELQLEQNFFNCSCDIRWMQLWQEQGEAKLNSQNLYCINADGSQLPLFRMNISQCDLPEISVSHVNLTVREGDNAVITCNGSGSPLPDVDWIVTGLQSINTHQTNLNWTNVHAINLTLVNVTSEDNGFTLTCIAENVVGMSNASVALTVYYPPRVVSLEEPELRLEHCIEFVVRGNPPPTLHWLHNGQPLRESKIIHVEYYQEGEISEGCLLFNKPTHYNNGNYTLIAKNPLGTANQTINGHFLKEPFPVDEVSPTPPITVTHKPEEDTFGVSIAVGLAAFACVLLVVLFVMINKYGRRSKFGMKGPVAVISGEEDSASPLHHINHGITTPSSLDAGPDTVVIGMTRIPVIENPQYFRQGHNCHKPDTYVQHIKRRDIVLKRELGEGAFGKVFLAECYNLSPTKDKMLVAVKALKDPTLAARKDFQREAELLTNLQHEHIVKFYGVCGDGDPLIMVFEYMKHGDLNKFLRAHGPDAMILVDGQPRQAKGELGLSQMLHIASQIASGMVYLASQHFVHRDLATRNCLVGANLLVKIGDFGMSRDVYSTDYYRLFNPSGNDFCIWCEVGGHTMLPIRWMPPESIMYRKFTTESDVWSFGVILWEIFTYGKQPWFQLSNTEVIECITQGRVLERPRVCPKEVYDVMLGCWQREPQQRLNIKEIYKILHALGKATPIYLDILG

(19) United States
(12) Patent Application Publication
(10) Pub. No.: US 2021/0132075 A1
(43) Pub. Date: May 6, 2021

(54) MATERIALS AND METHODS FOR DETECTING FUSION PROTEINS

(71) Applicant: Ventana Medical Systems, Inc., Tucson, AZ (US)

(72) Inventors: Richard Sheng Poe Huang, Morrisville, NC (US); Yen-Catherine Hoang Nguyen Le, Tucson, AZ (US); Ina Menzl, Tucson, AZ (US); Derek T. Smith, Tucson, AZ (US)

(21) Appl. No.: 17/248,266

(22) Filed: Jan. 15, 2021

Related U.S. Application Data
(63) Continuation of application No. PCT/EP2019/069185, filed on Jul. 17, 2019.
(60) Provisional application No. 62/699,618, filed on Jul. 17, 2018.

(51) Int. Cl. G01N 33/68; G01N 33/52; G01N 33/58; G01N 33/574

(52) U.S. Cl. CPC G01N 33/68; G01N 33/5748; G01N 33/581; G01N 33/52

(57) ABSTRACT

Methods for histochemical and cytochemical detection of oncogenic rearrangements of genes that result in expression of a fusion protein; materials, kits, and systems useful in such methods; and products resulting from performance of such methods are disclosed herein. At least two protein binding entities are provided: one targeting a portion of a wild-type protein that is retained in a fusion protein and a one targeting a portion of the wild type protein that is lost during the rearrangement that forms the fusion protein. A sample of a tissue suspected of harboring the fusion protein is stained with each of the two entities (either in simplex format or multiplex format), and the staining pattern resulting from binding of the entities is compared to determine the presence or absence of the fusion protein.

### CROSS-REFERENCE TO RELATED APPLICATIONS

This is a continuation of International Application No. PCT/EP2019/069185, filed Jul. 17, 2019, which claims priority to and the benefit of U.S. Provisional Patent Application No. 62/699,618, filed on Jul. 17, 2018, the content of each of which is incorporated herein by reference in its entirety.

### SEQUENCE LISTING INCORPORATION BY REFERENCE

This application hereby incorporates-by-reference a sequence listing submitted herewith in a computer-readable format, having a file name of P34909US1_Sequence_Listing_ST25, created on Jan. 14, 2021, which is 132,274 bytes in size.

### TECHNICAL FIELD

The present disclosure relates, among other things, to methods for the histochemical and cytochemical detection of oncogenic rearrangements of genes that result in expression of a fusion protein; materials, kits, and systems useful in such methods; and products resulting from performance of such methods.

### BACKGROUND

Fusion proteins have long been recognized as important drivers of oncogenic events and are frequent therapeutic targets. See Latsheva & Babu. However, a cheap and easy method for screening tumors for the presence of fusion proteins has remained elusive. Sequencing, RT-PCR, in situ hybridization, and immunohistochemistry have all been used to identify fusion events in tumors. See generally Bubendorf et al. (reviewing various methods used to identify fusions involving the ROS1 gene in non-small cell lung cancer). However, each has its limitations. Sequencing is expensive and loses spatial context, which may complicate the analysis. RT-PCR loses spatial context and may not be able to identify all potential rearrangements. In situ hybridization is time consuming and technically difficult to perform and interpret.

Immunohistochemistry targeting a domain of the wild-type protein that is retained in the fusion protein is cheap and can be easily performed and interpreted, but cannot distinguish between the fusion protein and the wild-type protein. There still remains a need for a cost-effective way of screening tumors for fusion proteins.

### SUMMARY

The application relates generally to materials and methods for the histochemical or cytochemical detection of a fusion protein using a first biomarker specific reagent that targets a retained portion of a wild-type counterpart protein involved in the fusion protein and a second biomarker specific reagent that targets a lost portion of the same wild-type counterpart protein.

In an embodiment, sets of biomarker-specific reagents that are provided that are capable of distinguishing between samples likely to express only a wild-type counterpart protein, samples that are likely to express a fusion protein involving the wild-type counterpart, and samples that are unlikely to express either the fusion protein or the wild-type counterpart.

In an embodiment, the sets of biomarker-specific reagents are used to stain samples suspected of harboring a fusion protein by affinity histochemical or affinity cytochemical methods.

In an embodiment, a set of stained samples generated by the presently disclosed methods are used to determine the presence or absence of a fusion protein in a patient sample.

In an embodiment, the assay as described herein is used to characterize a tumor sample from a patient.

In an embodiment, a kit is provided for performing the staining methods as described herein, the kit comprises a first biomarker-specific reagent and a second biomarker-specific reagent.

Other embodiments will be apparent from the following disclosure and the appended claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A illustrates an exemplary consensus retained portion and consensus lost portion of a theoretical group of C-terminal fusion proteins.

FIG. 1B illustrates an exemplary consensus retained portion and consensus lost portion of a theoretical group of N-terminal fusion proteins.

FIG. 1C illustrates an exemplary consensus retained portions and excluded region of a theoretical group of N- and C-terminal fusion proteins.

FIG. 2 illustrates an alignment between isoforms TrkA-I (SEQ ID NO: 12), TrkA-II (SEQ ID NO: 13), and TrkA Isoform 3 (SEQ ID NO: 4).

FIG. 3 illustrates an alignment between isoforms GP145-Trkb (SEQ ID NO: 14) and TrkB (Isoform 4) (SEQ ID NO: 5).

FIG. 4 illustrates an alignment between TrkC isoforms 1 (SEQ ID NO: 6), 3 (SEQ ID NO: 15), and 4 (SEQ ID NO: 16).

FIG. 5 illustrates an alignment between canonical amino acid sequences of TrkA (SEQ ID NO: 4), TrkB (SEQ ID NO: 5), and TrkC (SEQ ID NO: 6).

FIG. 6 illustrates an exemplary digital pathology system as disclosed herein.

FIG. 7 is an illustration of the OptiView DAB IHC Detection Kit.

FIG. 8 shows IHC stains using a reference dilution of a c-terminal specific ROS1 antibody and serial dilutions of an n-terminal specific ROS1 antibody.

FIG. 9 illustrates digital images of exemplary “fusion negative” cases (Case ID No. 1 and 3) stained with an C-terminal (left column) or an N-terminal (right column) Ros1 antibody. All images are captured at 2× magnification.

FIG. 10A illustrates digital images of exemplary “fusion positive” cases (Case ID No. 2, 4, and 5) stained with an C-terminal (left column) or an N-terminal (right column) Ros1 antibody. All images are captured at 2× magnification.

FIG. 10B illustrates digital images of exemplary “fusion positive” cases (Case ID No. 9, 6, and 7) stained with an C-terminal (left column) or an N-terminal (right column) Ros1 antibody. All images are captured at 2× magnification.

FIG. 10C illustrates digital images of exemplary “fusion positive” cases (Case ID No. 8) stained with an C-terminal (left column) or an N-terminal (right column) Ros1 antibody. All images are captured at 2× magnification.

### DETAILED DESCRIPTION

### I. Definitions

Unless defined otherwise, technical and scientific terms used herein have the same meaning as commonly understood by a person of ordinary skill in the art. See, e.g., Lackie, DICTIONARY OF CELL AND MOLECULAR BIOLOGY, Elsevier (4th ed. 2007); Sambrook et al., MOLECULAR CLONING, A LABORATORY MANUAL, Cold Springs Harbor Press (Cold Springs Harbor, N.Y. 1989). The term “a” or “an” is intended to mean “one or more.” The terms “comprise,” “comprises,” and “comprising,” when preceding the recitation of a step or an element, are intended to mean that the addition of further steps or elements is optional and not excluded.

Antibody: The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

Antibody fragment: An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)2; diabodies; linear antibodies; single-chain antibody molecules (e.g. scFv); and multispecific antibodies formed from antibody fragments.

Biomarker: As used herein, the term “biomarker” shall refer to any molecule or group of molecules found in a biological sample that can be used to characterize the biological sample or a subject from which the biological sample is obtained. For example, a biomarker may be a molecule or group of molecules whose presence, absence, or relative abundance is:
characteristic of a particular cell or tissue type or state;
characteristic of a particular pathological condition or state; or
indicative of the severity of a pathological condition, the likelihood of progression or regression of the pathological condition, and/or the likelihood that the pathological condition will respond to a particular treatment.

As another example, the biomarker may be a cell type or a microorganism (such as bacteria, mycobacteria, fungi, viruses, and the like), or a substituent molecule or group of molecules thereof.

Biomarker-specific reagent: A specific detection reagent that is capable of specifically binding directly to one or more biomarkers in the cellular sample, such as a primary antibody.

“C-terminal portion of Ros1” shall mean a retained portion of a wild-type Ros1 protein.

C-terminus fusion: A fusion protein in which the retained portion of the reference wild-type counterpart includes the c-terminus of the full-length wild-type counterpart.

Cellular sample: As used herein, the term “cellular sample” refers to any sample containing intact cells, such as cell cultures, bodily fluid samples or surgical specimens taken for pathological, histological, or cytological interpretation.

Consensus Lost Portion: A portion of a wild-type counterpart that is not contained in any of a defined group of fusion proteins having different breakpoints.

Consensus Retained Portion: A portion of a wild-type counterpart that is contained in each of a plurality of fusion proteins having different breakpoints.

Cytochemical detection: A process involving labelling biomarkers or other structures in a cytological sample with biomarker-specific reagents and detection reagents in a manner that permits microscopic detection of the biomarker or other structures in the context of intact cells.

Cytological sample: As used herein, the term “cytological sample” shall refer to a cellular sample that either have no cross-sectional spatial relationship in vivo (such as cellular samples derived from blood samples, urine samples, sputum, etc.) or in which the cross-sectional spatial relationship has been at least partially disrupted (such as tissue smears, liquid-based cytology samples, fine needle aspirates, etc.).

Detection reagent: A “detection reagent” is any reagent that is used to deposit a stain in proximity to a biomarker-specific reagent in a cellular sample. Non-limiting examples include biomarker-specific reagents (such as primary antibodies), secondary detection reagents (such as secondary antibodies capable of binding to a primary antibody), tertiary detection reagents (such as tertiary antibodies capable of binding to secondary antibodies), enzymes directly or indirectly associated with the biomarker specific reagent, chemicals reactive with such enzymes to effect deposition of a fluorescent or chromogenic stain, wash reagents used between staining steps, and the like.

Detectable moiety: A molecule or material that can produce a detectable signal (such as visually, electronically or otherwise) that indicates the presence (i.e. qualitative analysis) and/or concentration (i.e. quantitative analysis) of the detectable moiety deposited on a sample. A detectable signal can be generated by any known or yet to be discovered mechanism including absorption, emission and/or scattering of a photon (including radio frequency, microwave frequency, infrared frequency, visible frequency and ultra-violet frequency photons). The term “detectable moiety” includes chromogenic, fluorescent, phosphorescent, and luminescent molecules and materials, catalysts (such as enzymes) that convert one substance into another substance to provide a detectable difference (such as by converting a colorless substance into a colored substance or vice versa, or by producing a precipitate or increasing sample turbidity). In some examples, the detectable moiety is a fluorophore, which belongs to several common chemical classes including coumarins, fluoresceins (or fluorescein derivatives and analogs), rhodamines, resorufins, luminophores and cyanines. Additional examples of fluorescent molecules can be found in Molecular Probes Handbook—A Guide to Fluorescent Probes and Labeling Technologies, Molecular Probes, Eugene, Oreg., ThermoFisher Scientific, 11^{th} Edition. In other embodiments, the detectable moiety is a molecule detectable via brightfield microscopy, such as dyes including diaminobenzidine (DAB), 4-(dimethylamino) azobenzene-4′-sulfonamide (DAB SYL), tetramethylrhodamine (DISCOVERY Purple), N,N′-biscarboxypentyl-5,5′-disulfonato-indo-dicarbocyanine (Cy5), and Rhodamine 110 (Rhodamine).

Excluded region: A portion of a wild-type counterpart that is present in both: (a) a consensus retained portion of the N-terminus fusions of a defined group of fusion proteins involving the wild-type counterpart, and (b) a consensus retained portion of the C-terminus fusions of the same defined group of fusion proteins.

Histochemical detection: A process involving labelling biomarkers or other structures in a tissue sample with biomarker-specific reagents and detection reagents in a manner that permits microscopic detection of the biomarker or other structures in the context of the cross-sectional relationship between the structures of the tissue sample.

Intensity-matched staining: A set of staining conditions in which, for a wild-type sample, a stain intensity of a first section stained with a lost portion specific binding agent matches a staining intensity of a serial section of the first section stained with a retained portion specific binding agent.

Lost portion shall mean any portion of a wild-type counterpart of an oncogenic fusion protein that is not preserved in an oncogenic fusion protein. In an embodiment, the lost portion includes a portion of a wild-type counterpart protein that is not conserved in at least 50% of, at least 60% of, at least 75% of, at least 80% of, at least 85% of, at least 90% of, at least 91% of, at least 92% of, at least 93% of, at least 94% of, at least 95% of, at least 96% of, at least 97% of, at least 98% of, at least 99% of, or all know fusion proteins resulting from oncogenic rearrangement of the gene encoding the wild-type counterpart protein.

Monoclonal antibody: An antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, or a combination thereof.

N-terminal portion of Ros1 shall mean a lost portion of a wild-type Ros1 protein.

N-terminus fusion: A fusion protein in which the retained portion of the reference wild-type counterpart includes the N-terminus of the full-length wild-type counterpart.

Non-variant region: A portion of a wild-type counterpart that is retained in one or more alternate splice variant(s) of the wild-type counterpart.

“Retained portion” shall mean any portion of a wild-type counterpart of an oncogenic fusion protein that is preserved in the oncogenic fusion protein.

Simplex histochemical stain: A histochemical staining method in which a single biomarker-specific reagent is applied to a single section and stained with a single color stain.

Specific binding: As used herein, the phrase “specific binding,” “specifically binds to,” or “specific for” or other similar iterations refers to measurable and reproducible interactions between a target and a specific detection reagent, which is determinative of the presence of the target in the presence of a heterogeneous population of molecules including biological molecules. For example, an antibody that specifically binds to a target is an antibody that binds this target with greater affinity, avidity, more readily, and/or with greater duration than it binds to other targets. In one embodiment, the extent of binding of a specific detection reagent to an unrelated target is less than about 10% of the binding of the antibody to the target as measured, e.g., by a radioimmunoassay (RIA). In certain embodiments, a biomarker-specific reagent that specifically binds to a target has a dissociation constant (Kd) of ≤1 μM, ≤100 nM, ≤10 nM, ≤1 nM, or ≤0.1 nM. In another embodiment, specific binding can include, but does not require exclusive binding.

Specific detection reagent: Any composition of matter that is capable of specifically binding to a target chemical structure in the context of a cellular sample.

Stain: When used as a noun, the term “stain” shall refer to any substance that can be used to visualize specific molecules or structures in a cellular sample for microscopic analysis, including brightfield microscopy, fluorescent microscopy, electron microscopy, and the like. When used as a verb, the term “stain” shall refer to any process that results in deposition of a stain on a cellular sample.

Subject: As used herein, the term “subject” or “individual” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

Test sample: A tumor sample obtained from a subject having an unknown outcome at the time the sample is obtained.

Tissue sample: As used herein, the term “tissue sample” shall refer to a cellular sample that preserves the cross-sectional spatial relationship between the cells as they existed within the subject from which the sample was obtained.

Tumor sample: A tissue sample obtained from a tumor.

“Wild-type counterpart” shall mean any protein containing a retained portion of an oncogenic fusion protein that is expressed from a gene that has not undergone an oncogenic chromosomal rearrangement event.

“Wild-type sample” shall mean a sample that expresses a wild-type counterpart, and does not express the fusion protein.

### II. Background

The application relates generally to materials and methods for the histochemical or cytochemical detection of a fusion protein using a first biomarker specific reagent that targets a retained portion of a wild-type counterpart protein involved in the fusion protein and a second biomarker specific reagent that targets a lost portion of the same wild-type counterpart protein. The presence of the fusion protein is characterized by a reduction in the ability of the second biomarker specific reagent to bind to the sample relative to the first biomarker specific reagent. The absence of a fusion protein is characterized by similarity in the ability of the first and the second biomarker specific reagent to bind to the sample. Exemplary wild type counterpart proteins are set forth in Table 1:

| Wild-type | | |
|---|---|---|
| protein | Exemplary amino acid sequence | Background References |
| ROS1 | Uniprot P08922-1 (SEQ ID NO: 1) | Uguen & De Braekeleer; |
| | | Stransky et al. |
| RET | Uniprot P07949-1 (SEQ ID NO: 2) | Cascone et al.; Le Rolle et al.; |
| | | Stransky et al. |
| ALK | Uniprot Q9UM73-1 (SEQ ID NO: 3) | Ross et al. (2017); Stransky et al. |
| TrkA | Uniprot P04629-3 (SEQ ID NO: 4) | Amatu et al.; Stransky et al. |
| TrkB | Uniprot Q16620-4 (SEQ ID NO: 5) | Amatu et al.; Stransky et al. |
| TrkC | Uniprot Q16288-1 (SEQ ID NO: 6) | Amatu et al.; Stransky et al. |
| RAF1 | Uniprot P04049-1 (SEQ ID NO: 7) | Palanisamy et al.; Stransky et al. |
| BRAF | Uniprot P15056-1 (SEQ ID NO: 8) | Palanisamy et al.; Ross et al. |
| | | (2016); Stransky et al. |
| PRKCA | Uniprot P17252-1 (SEQ ID NO: 9) | Stransky et al. |
| PRKCB | Uniprot P05771-1 (SEQ ID NO: 10) | Stransky et al. |
| PKN1 | Uniprot Q16512-1 (SEQ ID NO: 11) | Stransky et al. |

This list is not intended to be exhaustive, and the present materials and methods may be useful for detection of fusion proteins involving other wild-type counterpart proteins that are not recited above. More fusion proteins implicated in tumors can be found in, for example, the COSMIC database, which is updated regularly. New fusions are also routinely reported in peer-reviewed publications.

### III. Biomarker-Specific Reagent Sets

The present materials and methods use sets of biomarker-specific reagents that are capable of distinguishing between samples that are likely to express only a wild-type counterpart protein, samples that are likely to express a fusion protein involving the wild-type counterpart, and samples that are unlikely to express either the fusion protein or the wild-type counterpart.

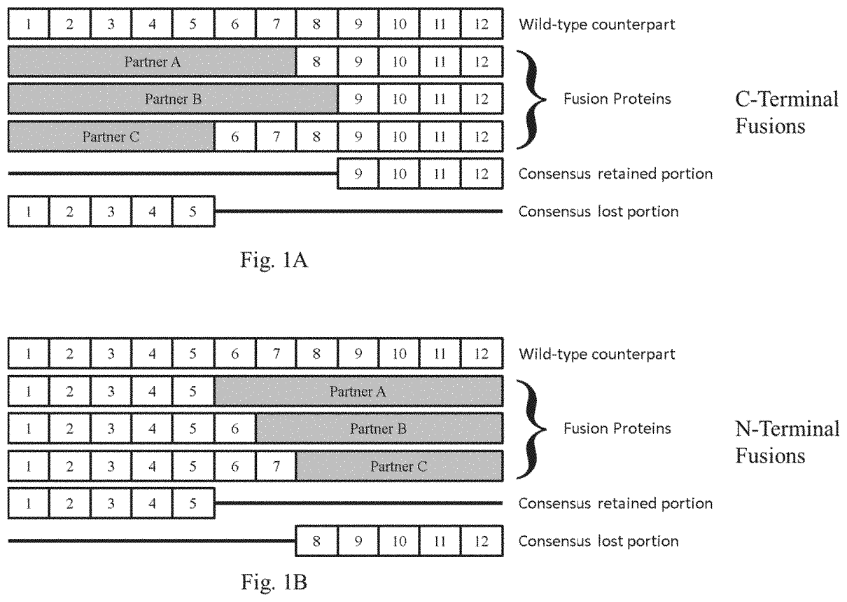

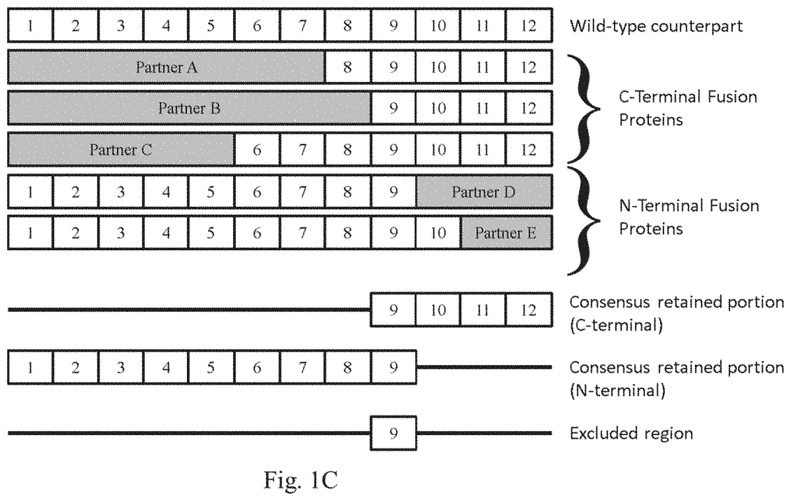

In another embodiment, the set of biomarker-specific reagents is selected by identifying the breakpoint of the wild-type protein that results in the fusion protein. A first biomarker-specific reagent for a retained portion is selected to target a portion of the wild-type protein on the side of the breakpoint retained in the fusion protein, while the biomarker-specific reagent for the lost portion is selected to target a portion of the wild-type counterpart on the side of the breakpoint that is not retained in the fusion protein. Many resources are available for identifying fusion protein breakpoints including, for example, the COSMIC database, which includes notations of breakpoints by genomic location and by the first exon expressed in the resulting fusion protein. For example, in one such embodiment, a first biomarker-specific reagent is selected to target a consensus retained portion and a second biomarker-specific reagent is selected to target a consensus lost portion for a defined group of fusions proteins involving the same wild-type counterpart, wherein at least some of the group of fusion proteins have different breakpoints. The consensus retained portion and the consensus lost portion may be identified by, for example, comparing the first exon of the wild-type counterpart expressed in each of the fusion proteins of the defined group. For C-terminus fusions: (a) the consensus retained portion is the portion of the wild-type counterpart starting with the N-terminal amino acid encoded by the first exon preserved in each of the fusion proteins of the defined group, and ending with the C-terminal amino acid of the wild-type counterpart; and (b) the consensus lost portion is the portion of the wild-type counterpart starting with the N-terminal amino acid of the wild-type counterpart and ending with the C-terminal amino acid encoded by the last exon lost in each of the fusion proteins of the defined group. For N-terminus fusions: (a) the consensus retained portion is the portion of the wild-type counterpart starting with the N-terminal amino acid of the wild-type counterpart and ending with the C-terminal amino acid encoded by the last exon lost in each of the fusion proteins of the defined group; and (b) the consensus lost portion is the portion of the wild-type counterpart starting with the N-terminal amino acid encoded by the first exon preserved in each of the fusion proteins of the defined group, and ending with the C-terminal amino acid of the wild-type counterpart. This is illustrated at FIGS. 1A and 1B. For the C-terminal fusions illustrated at FIG. 1A, three different fusion partners result in three different breakpoints: for fusions with Partner A, the breakpoint results in a fusion protein in which Exon 8 of the wild-type counterpart is the first expressed; for fusions with Partner B, the breakpoint results in a fusion protein in which Exon 9 of the wild-type counterpart is the first expressed; for fusions with Partner C, the breakpoint results in a fusion protein in which Exon 6 of the wild-type counterpart is the first expressed. Thus, the consensus retained portion includes a polypeptide expressed from Exons 9-12 because this is the portion of the wild-type counterpart that is retained in all three fusion proteins. And, the consensus lost portion includes a polypeptide expressed from Exons 1-5, because this is the portion of the wild-type counterpart that is absent from all three fusion proteins. For the N-terminal fusions illustrated at FIG. 1B, three different fusion partners result in three different breakpoints: for fusions with Partner A, the breakpoint results in a fusion protein in which Exon 5 of the wild-type counterpart is the last expressed; for fusions with Partner B, the breakpoint results in a fusion protein in which Exon 6 of the wild-type counterpart is the last expressed; for fusions with Partner C, the breakpoint results in a fusion protein in which Exon 7 of the wild-type counterpart is the last expressed. Thus, the consensus retained portion includes a polypeptide expressed from Exons 1-5 because this is the portion of the wild-type counterpart that is retained in all three fusion proteins. Likewise, the consensus lost portion includes a polypeptide expressed from Exons 8-12, because this is the portion of the wild-type counterpart that is absent from all three fusion proteins. Exemplary breakpoints, consensus retained portions, and consensus lost portions for C-terminus fusions involving some wild-type counterparts are illustrated in Table 2:

TABLE 2

| Wild- | | First | | |
|---|---|---|---|---|
| type | Fusion | Observed | Consensus | Consensus Lost |
| protein | Partner | Exon | Retained Portion | Portion |
| ROS1 | CD74 | 34‡ | Exon 36 through | N-terminus |
| | | 32‡ | C-terminus | through Exon 31 |
| | CEP85L | 36* | | |
| | CLIP1 | 36‡ | | |
| | ERC1 | 36‡ | | |
| | EZR | 34‡ | | |
| | GOPC | 35‡ | | |
| | | 36‡ | | |
| | HLA-A | 34‡ | | |
| | KIAA1598 | 36‡ | | |
| | LRIG3 | 35‡ | | |
| | MYO5A | 35‡ | | |
| | PPFIBP1 | 35‡ | | |
| | PWWP2A | 36‡ | | |
| | SDC4 | 32‡ | | |
| | | 34‡ | | |
| | SLC34A2 | 32‡ | | |
| | TPM3 | 36‡ | | |
| | | 35‡ | | |
| | ZCCHC8 | 36‡ | | |
| RET | AKAP13 | 12* | Exon 12 through | N-terminus |
| | CCDC6 | 12‡ | C-terminus | through Exon 6 |
| | | 11‡ | | |
| | ERC1 | 12* | | |
| | FKBP15 | 12* | | |
| | GOLGA5 | 12‡ | | |
| | HOOK3 | 12‡ | | |
| | KIF5B | 12‡ | | |
| | | 8‡ | | |
| | | 11‡ | | |
| | | 7‡ | | |
| | KTN1 | 12‡ | | |
| | NCOA4 | 12‡ | | |
| | | 11‡ | | |
| | PCM1 | 12‡ | | |
| | PRKAR1A | 12‡ | | |
| | SPECC1L | 12* | | |
| | TBL1XR1 | 12* | | |
| | TRIM24 | 12‡ | | |
| | TRIM27 | 12‡ | | |
| | TRIM33 | 12‡ | | |
| ALK | ATIC | 20‡ | Exon 20 through | N-terminus |
| | C2orf44 | 20‡ | C-terminus | through Exon 1 |
| | CARS | 20‡ | | |
| | CLTC | 20‡ | | |
| | DCTN1 | 20‡ | | |
| | EML4 | 20*‡ | | |
| | | 19‡ | | |
| | | 17* | | |
| | FN1 | 19‡ | | |
| | GTF2IRD1 | 20* | | |
| | HIP1 | 20‡ | | |
| | KIF5B | 20‡ | | |
| | | 19‡ | | |
| | KLC1 | 20‡ | | |
| | MSN | 20‡ | | |
| | NPM1 | 20‡ | | |
| | PPFIBP1 | 20‡ | | |
| | RANBP2 | 20‡ | | |
| | SEC31A | 20‡ | | |
| | SMEK2 | 2* | | |
| | SQSTM1 | 20‡ | | |
| | STRN | 20*‡ | | |
| | TFG | 20‡ | | |
| | TPM1 | 20* | | |
| | TPM3 | 20‡ | | |
| | TPM4 | 20‡ | | |
| | VCL | 20‡ | | |
| TrkA | LMNA | 10‡ | Exon 10 through | N-terminus |
| | TFG | 6‡ | C-terminus | through Exon 5 |
| | TP53 | 9‡ | | |
| | TPM3 | 10‡ | | |
| TrkB | AFAP1 | 12* | Exon 17 through | N-terminus |
| | NACC2 | 13‡ | C-terminus | through Exon 12 |
| | QKI | 16‡ | | |
| | PAN3 | 17* | | |
| | SQSTM | 16 | | |
| | | (terminates | | |
| | | at exon 20)* | | |
| | TRIM24 | 15* | | |
| TrkC | ETV6 | 15‡ | Exon 15 through | N-terminus |
| | | 14‡ | C-terminus | through Exon 13 |
| RAF1 | AGGF1 | 8* | Exon 10 through | N-terminus |
| | CLCN6 | 8* | C-terminus | through Exon 5 |
| | ESRP1 | 6‡ | | |
| | HACL1 | 8‡ | | |
| | LMNA | 8* | | |
| | MPRIP | 8* | | |
| | PAPD7 | 10* | | |
| | MPRIP | 8* | | |
| | SRGAP3 | 10‡ | | |
| | | 8‡ | | |
| | TRAK1 | 8* | | |
| BRAF | AGTRAP | 8‡ | Exon 11 through | N-terminus |
| | AKAP9 | 9‡ | C-terminus | through Exon 6 |
| | AP3B1 | 9* | | |
| | ATG7 | 9* | | |
| | BCL2L11 | 10* | | |
| | CEP89 | 9‡ | | |
| | CLCN6 | 11‡ | | |
| | FAM131B | 9‡ | | |
| | | 10‡ | | |
| | FAM114A2 | 11* | | |
| | FCHSD1 | 9‡ | | |
| | GATM | 11‡ | | |
| | GNAI1 | 10‡ | | |
| | HERPUD1 | 7‡ | | |
| | KDM7A | 11* | | |
| | KIAA1549 | 9‡ | | |
| | | 11‡ | | |
| | | 10‡ | | |
| | LSM14A | 9‡ | | |
| | MKRN1 | 11‡ | | |
| | RNF130 | 9‡ | | |
| | SLC45A3 | 8‡ | | |
| | SND1 | 9‡ | | |
| | | 11‡ | | |
| | ZC3HAV1 | 11* | | |
| | ZSCAN30 | 10‡ | | |
| PRKCA | IGF2BP3 | 4* | Exon 6 through | N-terminus |
| | TANC2 | 6* | C-terminus | through Exon 3 |
| PRKCB | SPNS1 | 3* | Exon 3 through | N-terminus |
| | ADCY9 | 3* | C-terminus | through Exon 2 |
| | GGA2 | 3* | | |
| PKN1 | ANXA4 | 13* | Exon 13 through | N-terminus |
| | TECR | 10* | C-terminus | through Exon 9 |

*Breakpoint/first exon obtained from Stransky et al.
‡Breakpoint/first exon obtained from COSMIC database (as of Mar. 29, 2018)

Exemplary breakpoints, consensus retained portions, and consensus lost portions for N-terminus fusions involving some wild-type counterparts are illustrated in Table 3:

TABLE 3

| Wild- | | Last | | |
|---|---|---|---|---|
| type | Fusion | Observed | Consensus Retained | Consensus Lost |
| protein | Partner | Exon | Portion | Portion |
| RET | GOLGA5 | 11‡ | N-terminus through | Exon 12 through C- |
| | NCOA4 | 11‡ | Exon 11 | terminus |
| | TRIM33 | 11‡ | | |
| ALK | MSN | 20‡ | N-terminus through | Exon 21 through C- |
| | | | Exon 20 | terminus |
| TrkA | TFG | 5‡ | N-terminus through | Exon 10 through C- |
| | TPM3 | 9‡ | Exon 5 | terminus |
| TrkC | ETV6 | 14‡ | N-terminus through | Exon 15 through C- |
| | | | Exon 14 | terminus |
| RAF1 | ESRP1 | 5‡ | N-terminus through | Exon 8 through C- |
| | COSF676 | 7‡ | Exon 5 | terminus |
| BRAF | AKAP9 | 8‡ | N-terminus through | Exon 9 through C- |
| | | | Exon 8 | terminus |

‡Breakpoint/last exon obtained from COSMIC database (as of Mar. 29, 2018)

In another embodiment, both N-terminus fusions and C-terminus fusions involving the same wild-type counterpart are evaluated for consensus retained region(s) and excluded region(s), and a first biomarker-specific reagent is selected that is specific for a portion of the wild-type counterpart within a consensus retained region of the N-terminus fusions but not within the excluded region, and a second biomarker-specific reagent is selected that is specific for a portion of the wild-type counterpart within a consensus retained region of the C-terminus fusions but not within the excluded region. This is illustrated at FIG. 1C. For the C-terminal fusions illustrated at FIG. 1C, three different fusion partners result in three different breakpoints: for fusions with Partner A, the breakpoint results in a fusion protein in which Exon 8 of the wild-type counterpart is the first expressed; for fusions with Partner B, the breakpoint results in a fusion protein in which Exon 9 of the wild-type counterpart is the first expressed; for fusions with Partner C, the breakpoint results in a fusion protein in which Exon 6 of the wild-type counterpart is the first expressed. Thus, the consensus retained portion includes a polypeptide expressed from Exons 9-12 because this is the portion of the wild-type counterpart that is retained in all three fusion proteins. For the N-terminal fusions illustrated at FIG. 1C, two different fusion partners result in two different breakpoints: for fusions with Partner D, the breakpoint results in a fusion protein in which Exon 9 of the wild-type counterpart is the last expressed; for fusions with Partner E, the breakpoint results in a fusion protein in which Exon 10 of the wild-type counterpart is the last expressed. Thus, the consensus retained portion includes a polypeptide expressed from Exons 1-9 because this is the portion of the wild-type counterpart that is retained in both fusion proteins. As can be seen, if the one of the biomarker-specific agents is specific for exon 9, then fusions proteins will not be detectable, because both the first and the second biomarker-specific will bind to the fusion. Exemplary consensus retained portions and excluded portions for some wild-type counterparts are illustrated in Table 4:

TABLE 4

| Wild-type | Consensus Retained Portion | | Excluded |
|---|---|---|---|
| protein | (From Tables 2 & 3) | | Portion |
| RET | N-terminus | N-terminus through Exon 11 | None |
| | C-Terminus | Exon 12 through C-terminus | |
| ALK | N-terminus | N-terminus through Exon 20 | Exon 20 |
| | C-Terminus | Exon 20 through C-terminus | |
| TrkA | N-terminus | N-terminus through Exon 5 | None |
| | C-Terminus | Exon 10 through C-terminus | |
| TrkC | N-terminus | N-terminus through Exon 14 | None |
| | C-Terminus | Exon 15 through C-terminus | |
| RAF1 | N-terminus | N-terminus through Exon 5 | None |
| | C-Terminus | Exon 10 through C-terminus | |
| BRAF | N-terminus | N-terminus through Exon 8 | None |
| | C-Terminus | Exon 11 through C-terminus | |

The defined group in the foregoing embodiments does not need to include every known fusion protein including the wild-type counterpart, or every known breakpoint. Rather, in some embodiments, a subset of known fusions is selected. For example, the subset may be selected as representative of the fusions found in a particular tumor type, or as representative of the fusions most likely to be encountered in clinical practice. For example, the COSMIC database reports the total number of specific samples in which a fusion between a wild-type counterpart and a specific fusion partner has been observed. This number is used to select a representative sampling of fusions for analysis of breakpoints and selection of biomarker-specific reagents. For example, a defined group may be selected to represent at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or all fusions involving a specific wild-type counterpart reported in a specific database (such as the COSMIC database). As another example, a defined group may be selected to represent at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or all tumors bearing a fusion protein involving a specific wild-type counterpart reported in a specific database (such as the COSMIC database). As another example, a defined group may be selected to represent at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or all tumors of a specified primary tissue type (such as lung, colorectal, breast, urinary, skin, brain, prostate, etc.) or a subtype thereof (such as non-small cell lung, small cell lung, HER+ breast, triple-negative breast, etc.) bearing a fusion protein involving a specific wild-type counterpart reported in a specific database (such as the COSMIC database). As another example, a defined group may be selected to represent at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or all tumors of a specified histological type (such as carcinoma, sarcoma, myeloma, leukemia, lymphoma, or mixed types) or a histological subtype thereof (such as adenocarcinoma, squamous cell carcinoma, osteosarcoma, chondrosarcoma, leiomyosarcoma, rhabdomyosarcoma, mesothelioma, fibrosarcoma, angiosarcoma, hemangioendothelioma, liposarcoma, glioma, astrocytoma, myxosarcoma, mesenchymous or mixed mesodermal tumor, adenosquamous carcinoma, mixed mesodermal tumor, carcinosarcoma, or teratocarcinoma) bearing a fusion protein involving a specific wild-type counterpart reported in a specific database (such as the COSMIC database). As another example, the defined group may be selected to include the top 2, top 3, top 4, top 5, etc. most prevalent fusions involving a specific wild-type counterpart reported in a specific database (such as the COSMIC database).

In another embodiment, a first biomarker-specific reagent is selected that is capable of binding to an N-terminal portion of the wild-type counterpart, and a second biomarker-specific reagent that is capable of binding to a C-terminal portion of the wild-type counterpart. For C-terminus fusions, the first biomarker-specific reagent is used to detect the lost portion while the second biomarker-specific reagent is used to detect the retained portion. For N-terminus fusions, the first biomarker-specific reagent is used to detect the retained portion while the second biomarker-specific reagent is used to detect the lost portion. For wild-type proteins involved in both C-terminus and N-terminus fusions, either biomarker-specific reagent performs either function, depending on which fusion is present in the sample. It is not necessary to know which biomarker-specific reagent performs which function before performing the assay, so long as intensity-matched staining can be achieved for samples that express the wild-type counterpart, and differences can be observed in samples that express a fusion protein. In one example, the N- and C-terminal portions targeted by the biomarker-specific reagents comprise or consist of a region that is less than 300 amino acids from the respective terminus. In one example, the N- and C-terminal portions targeted by the biomarker-specific reagents comprise or consist of a region that is less than 200 amino acids from the respective terminus. In one example, the N- and C-terminal portions targeted by the biomarker-specific reagents comprise or consist of a region that is less than 100 amino acids from the respective terminus. In another example, the N- and C-terminal portions targeted by the biomarker-specific reagents comprise or consist of a region that is less than 75 amino acids from the respective terminus. In another example, the N- and C-terminal portions targeted by the biomarker-specific reagents comprise or consist of a region that is less than 50 amino acids from the respective terminus. In another example, the N- and C-terminal portions targeted by the biomarker-specific reagents comprise or consist of a region that is less than 40 amino acids from the respective terminus. In another example, the N- and C-terminal portions targeted by the biomarker-specific reagents comprise or consist of a region that is less than 35 amino acids from the respective terminus. In another example, the N- and C-terminal portions targeted by the biomarker-specific reagents comprise or consist of a region that is less than 30 amino acids from the respective terminus. In another example, the fragments of the wild-type counterparts are less than 25 amino acids in length. In another example, the N- and C-terminal portions targeted by the biomarker-specific reagents comprise or consist of a region that is less than 20 amino acids from the respective terminus. Specific examples of N-terminus and C-terminus directed antibody combinations are disclosed in Table 5:

TABLE 5

| | Antibodies | | | | |
|---|---|---|---|---|---|
| Wild- | N- or | | | | |
| type | C- | Antibody or | Supplier | Clonality & | |
| protein | term.? | Clone name | (Cat. No.) | isotype | Epitope/Immunogen |
| ROS1 | N- | 4-6G | Abcam plc | Mouse | 1-285 of SEQ |
| | term. | | (ab108492) | monoclonal | ID NO: 1 |
| | | | | IgG1 | |
| | | 5-7H | Creative | Mouse | 1-285 of SEQ |
| | | | Diagnostics | monoclonal | ID NO: 1 |
| | | | (CABT- | IgG1 | |
| | | | 34802MH) | | |
| | | Anti-ROS1/ | LifeSpan | Rabbit | 23-56 of SEQ |
| | | ROS Antibody | BioSciences, Inc. | polyclonal | ID NO: 1 |
| | | (aa23-56) | (LS-C339686) | | |
| | | anti-ROS1 | antiobodies- | Rabbit | 33-63 of SEQ |
| | | Antibody | online.com | polyclonal | ID NO: 1 |
| | | | (ABIN2579391) | | |
| | | ROS1 Polyclonal | ThermoFisher | Rabbit | 39-57 of SEQ |
| | | Antibody | Scientific | polyclonal | ID NO: 1 |
| | | | (PA1-30318) | IgG | |
| | C- | EPMGHR2 | Abcam plc | Rabbit | 2050-2150 of |
| | term. | | (ab189925) | monoclonal | SEQ ID NO: 1 |
| | | | | IgG | |
| | | 1F6 | LifeSpan | Mouse | 2126-2347 of |
| | | | BioSciences, Inc. | monoclonal | SEQ ID NO: 1 |
| | | | (LS-C339686) | IgG2b | |
| | | 2A8 | LifeSpan | Mouse | 2126-2347 of |
| | | | BioSciences, Inc. | monoclonal | SEQ ID NO: 1 |
| | | | (LS-C340434) | IgG2b | |
| | | 5D1 | LifeSpan | Mouse | 2126-2347 of |
| | | | BioSciences, Inc. | monoclonal | SEQ ID NO: 1 |
| | | | (LS-C340436) | IgG2b | |
| | | 1F3 | LifeSpan | Mouse | 2126-2347 of |
| | | | BioSciences, Inc. | monoclonal | SEQ ID NO: 1 |
| | | | (LS-C339688) | IgG1 | |
| | | 3F12 | LifeSpan | Mouse | 2126-2347 of |
| | | | BioSciences, Inc. | monoclonal | SEQ ID NO: 1 |
| | | | (LS-C340467) | IgG2a | |
| | | 4A4 | LifeSpan | Mouse | 2126-2347 of |
| | | | BioSciences, Inc. | monoclonal | SEQ ID NO: 1 |
| | | | (LS-C339703) | IgG1 | |
| | | c-Ros Polyclonal | Bioss Inc. | Rabbit | 2300-2345/47 |
| | | Antibody | (bs-2504R) | polyclonal | of SEQ ID NO: |
| | | | | IgG | 1 |
| | | ROS1 (D4D6 ®)) | Cell Signaling | Rabbit | Unspecified |
| | | Rabbit mAb | Technology, Inc. | monoclonal | residues |
| | | | (#3287) | IgG | residing in |
| | | | | | carboxy |
| | | | | | terminal |
| | | | | | domain |
| RET | N- | 1A5 | LifeSpan | Mouse | 361-458 of |
| | term. | | BioSciences, Inc. | monoclonal | SEQ ID NO: 2 |
| | | | (LS-B10954) | IgG2a,k | |
| | | 4B7 | Creative | Mouse | 361-458 of |
| | | | Diagnostics | monoclonal | SEQ ID NO: 2 |
| | | | (DCABH-13226) | IgG2a | |
| | | Anti-Ret | Creative | Rabbit | Unspecified |
| | | polyclonal | Diagnostics | polyclonal | sequence |
| | | antibody | (DCABH-11989) | IgG | corresponding |
| | | | | | to human Ret |
| | | | | | N-terminus |
| | | E1N8X | Cell Signaling | Rabbit | Unspecified |
| | | | Technology, Inc. | monoclonal | peptide |
| | | | (#14556) | IgG | surrounding |
| | | | | | Pro320 of SEQ |
| | | | | | ID NO: 2 |
| | | C-3 | Santa Cruz | Mouse | 31-330 of SEQ |
| | | | Biotechnology Inc. | monoclonal | ID NO: 2 |
| | | | (sc-365943) | IgG1k | |
| | | RET Polyclonal | ThermoFisher | Rabbit | 152-182 of |
| | | Antibody | Scientific | polyclonal | SEQ ID NO: 2 |
| | | | (PAS-14722) | IgG | |
| | | N-term Q28 | RayBiotech, Inc. | Rabbit | 13-44 of SEQ |
| | | | (102-17541) | polyclonal Ig | ID NO: 2 |
| | C- | RET01 | LifeSpan | Mouse | Unspecified |
| | term. | | BioSciences, Inc. | monoclonal | residues in the |
| | | | (LS-C95523) | IgG1 | extreme C- |
| | | | | | terminal |
| | | | | | cytoplasmic |
| | | | | | region |
| | | 8D10C9 | LifeSpan | Mouse | aa896-1063 of |
| | | | BioSciences, Inc. | monoclonal | SEQ ID NO: 2 |
| | | | (LS-B6328) | IgG1 | |
| | | Anti-RET | LifeSpan | Mouse | aa896-1063 of |
| | | Antibody | BioSciences, Inc. | monoclonal | SEQ ID NO: 2 |
| | | | (LS-C41512) | IgG1 | |
| | | 5D4 | LifeSpan | Mouse | aa713-1017 of |
| | | | BioSciences, Inc. | monoclonal | SEQ ID NO: 2 |
| | | | (LS-C41512) | IgG1 | |
| | | 3F8 | LifeSpan | Mouse | Unspecified |
| | | | BioSciences, Inc. | monoclonal | residues in the |
| | | | (LS-C87551) | IgG1 | extreme C- |
| | | | | | terminal |
| | | | | | cytoplasmic |
| | | | | | region |
| | | 1G1 | LifeSpan | Mouse | aa713-1017 of |
| | | | BioSciences, Inc. | monoclonal | SEQ ID NO: 2 |
| | | | (LS-C339712) | IgG1 | |
| | | EPR2871 | Abcam plc | Mouse | Unspecified |
| | | | (ab134100) | monoclonal | peptide within |
| | | | | IgG1 | C-terminus |
| | | 9E21D0 | Creative | Mouse | 896-1063 of |
| | | | Diagnostics | monoclonal | SEQ ID NO: 2 |
| | | | (DCABH-1947) | IgG1 | |
| | | 6E4C4 | Santa Cruz | Mouse | 896-1063 of |
| | | | Biotechnology Inc. | monoclonal | SEQ ID NO: 2 |
| | | | (sc-101423) | IgG1k | |
| | | 8D10C9 | Santa Cruz | Mouse | 896-1063 of |
| | | | Biotechnology Inc. | monoclonal | SEQ ID NO: 2 |
| | | | (sc-101422) | IgG1k | |
| ALK | N- | RB1511-1512 | Aviva Systems | Rabbit | 14-43 of SEQ |
| | term. | | Biology Corp. | polyclonal Ig | ID NO: 3 |
| | | | (OAAB20861) | | |
| | C- | Ab-1586 | Aviva Systems | Rabbit | Non- |
| | term. | | Biology Corp. | polyclonal | phosphopeptide |
| | | | (OAAB20861) | IgG | around Y1586 |
| | | | | | of SEQ ID NO: |
| | | | | | 3 |
| | | 5A4 | Leica | Mouse | 1359-1460 of |
| | | | (NCL-L-ALK) | monoclonal | SEQ ID NO: 3 |
| | | | | IgG | |
| | | SP8 | Abcam | Rabbit | 1366-1468 of |
| | | | (ab16670) | monoclonal | SEQ ID NO: 3 |
| | | | | IgG | |
| | | D5F3 | Cell Signaling | Rabbit | Recombinant |
| | | | Technology, Inc. | monoclonal | protein |
| | | | (#3633) | IgG | corresponding |
| | | | | | to |
| | | | | | residues in the |
| | | | | | carboxy |
| | | | | | terminus of |
| | | | | | human ALK |
| | | ALK1 | Agilent | Mouse | 1359-1460 of |
| | | | Technologies | monoclonal | SEQ ID NO: 3 |
| | | | (M719529-2) | IgG3, kappa | |
| TrkA | N- | Y32Ex | Santa Cruz | Mouse | Raised against |
| | term. | | Biotechnology Inc. | monoclonal | extracellular |
| | | | (sc-80398) | IgG2a, kappa | domain of |
| | | | | light chain | TrkA of human |
| | | | | | origin |
| | | 165126 | R&D Systems, | Mouse | 4-377 of SEQ |
| | | | Inc. | monoclonal | ID NO: 4 |
| | | | | IgG2A | |
| | C- | B-3 | Santa Cruz | Mouse | 747-760 of |
| | term. | | Biotechnology Inc. | monoclonal | SEQ ID NO: |
| | | | (sc-7268) | IgG2a | 4* |
| | | 5B6 | LifeSpan | Mouse | 404-760 of |
| | | | BioSciences, Inc. | monoclonal | SEQ ID NO: 4 |
| | | | (LS-C339966) | IgG1 | |
| | | EPR17341 | Abcam | Rabbit | 816-838 of |
| | | | (ab181560) | monoclonal | SEQ ID NO: |
| | | | | IgG | 5* |
| TrkB | N- | 75133 | LifeSpan | Mouse | 32-430 of SEQ |
| | term. | | BioSciences, Inc. | monoclonal | ID NO: 5 |
| | | | (LS-C150091) | IgG2b | |
| | | 10B6C4 | Novus Biologicals | Mouse | 207-339 of |
| | | | (NBP2-52524) | monoclonal | SEQ ID NO: 5 |
| | | | | IgG1 | |
| | C- | EPR17341 | Abcam | Rabbit | 816-838 of |
| | term. | | (ab181560) | monoclonal | SEQ ID NO: |
| | | | | IgG | 5* |
| | | B-3 | Santa Cruz | Mouse | 747-760 of |
| | | | Biotechnology Inc. | monoclonal | SEQ ID NO: |
| | | | (sc-7268) | IgG2a | 4* |
| TrkC | N- | 7H6 | LifeSpan | Mouse | 32-429 of SEQ |
| | term. | | BioSciences, Inc. | monoclonal | ID NO: 6 |
| | | | (LS-C108850) | IgG2b | |
| | | WW6 | Santa Cruz | Mouse | Extracellular |
| | | | Biotechnology Inc. | monoclonal | domain of |
| | | | (sc-80403) | IgG2b | TrkC of human |
| | | | | | origin |
| | | 8I7 | Creative | Mouse IgG1 | 32-429 of SEQ |
| | | | Diagnostics | | ID NO: 6 |
| | | | (DCABH-12634) | | |
| | | Anti-NTRK3/ | LifeSpan | Rabbit | 300-400 of |
| | | TRKC Antibody | BioSciences, Inc. | polyclonal | SEQ ID NO: 6 |
| | | (aa300-400) | (LS-C359307) | | |
| | | Anti-NTRK3/ | LifeSpan | Rabbit | 31-61 of SEQ |
| | | TRKC Antibody | BioSciences, Inc. | polyclonal | ID NO: 6 |
| | | (aa31-61) IHC- | (LS-B10709) | | |
| | | plus ™ | | | |
| | C- | EPR17341 | Abcam | Rabbit | 816-838 of |
| | term. | | (ab181560) | monoclonal | SEQ ID NO: |
| | | | | IgG | 5* |
| | | B-3 | Santa Cruz | Mouse | 747-760 of |
| | | | Biotechnology Inc. | monoclonal | SEQ ID NO: |
| | | | (sc-7268) | IgG2a | 4* |
| RAF1 | N- | Anti-RAF1/ | LifeSpan | Rabbit | 1-240 of SEQ |
| | term. | RAF Antibody | BioSciences, Inc. | monoclonal | ID NO: 7 |
| | | (N-Terminus) | (LS-B6239) | IgG1, k | |
| | | IHC-plus ™ | | | |
| | C- | C-10 | Santa Cruz | Mouse | 621-655 of |
| | term. | | Biotechnology Inc. | monoclonal | SEQ ID NO: 7 |
| | | | (sc-373722) | IgG1 (kappa | |
| | | | | light chain) | |
| | | E-10 | Santa Cruz | Mouse | 637-648 of |
| | | | Biotechnology Inc. | monoclonal | SEQ ID NO: 7 |
| | | | (sc-7267) | IgG1 (kappa | |
| | | | | light chain) | |
| | | H-8 | Santa Cruz | Mouse | 621-655 of |
| | | | Biotechnology Inc. | monoclonal | SEQ ID NO: 7 |
| | | | (sc-376142) | IgG3 (kappa | |
| | | | | light chain) | |
| BRAF | N- | RM308 | NSJ Bioreagents | Rabbit | N-terminus of |
| | term. | | (R20328-0) | monoclonal | human B-raf |
| | | | | IgG | |
| | C- | Anti-BRAF/B- | LifeSpan | Rabbit | C-terminal |
| | term. | Raf Antibody (C- | BioSciences, Inc. | polyclonal | region of B-raf |
| | | Terminus) | (LS-C353922) | | |
| | | C-19 | Santa Cruz | Rabbit | C-terminal |
| | | | Biotechnology Inc. | polyclonal | region of B-raf |
| | | | (sc-166) | IgG | |
| PRKCA | N- | Anti-PRKCA | LifeSpan | Rabbit | 1-30 of SEQ |
| | term. | PKC-Alpha | BioSciences, Inc. | polyclonal | ID NO: 9 |
| | | Antibody (aa1- | (LS-B14519) | IgG | |
| | | 30) IHC-plus ™ | | | |
| | C- | 133 | Abcam | Mouse | 661-672 of |
| | term. | | (ab11723) | monoclonal | SEQ ID NO: 9 |
| | | | | IgG2a | |
| | | ANTI-PKC | Sigma-Aldrich | Rabbit | C-terminus of |
| | | ALPHA | (SAB1305634) | polyclonal | human PRKCA |
| | | | | IgG | |
| | | EPR16794 | Abcam | Rabbit | 450 through C- |
| | | | (ab179521) | monoclonal | terminus of |
| | | | | IgG | SEQ ID NO: |
| | | | | | 10 |
| | | | | | (Specificity for |
| | | | | | PRKCA and |
| | | | | | PRKCB) |
| PRKCB | N- | ARP56423_P050 | Aviva Systems | Rabbit | 1-50 of SEQ |
| | term. | | Biology | polyclonal | ID NO: 10 |
| | | | (ARP56423_P050) | IgG | |
| | | Anti-PKC beta | Abcam | Rabbit | 1-270 of SEQ |
| | | 1 + PKC beta 2 | (ab189782) | polyclonal | ID NO: 10 |
| | | antibody-N- | | | |
| | | terminal | | | |
| | | Anti-PKC beta 1 | Abcam | Rabbit | 1-30 of SEQ |
| | | N-terminal | (ab189782) | polyclonal | ID NO: 10 |
| | C- | EPR16794 | Abcam | Rabbit | 450 through C- |
| | term. | | (ab179521) | monoclonal | terminus of |
| | | | | IgG | SEQ ID NO: |
| | | | | | 10 (Specificity |
| | | | | | for PRKCA |
| | | | | | and PRKCB) |
| | | EPR18512 | Abcam | Rabbit | 600 through C- |
| | | | (ab195039) | monoclonal | terminus of |
| | | | | IgG | SEQ ID NO: |
| | | | | | 10 |
| | | A10-F | Novus Biologicals | Rabbit | 658-666 of |
| | | | (NBP1-30122) | monoclonal | SEQ ID NO: |
| | | | | IgG | 10 |
| | | PRKCB | ProSci Inc. | Goat | 631-642 of |
| | | Antibody | (43-319) | polyclonal | SEQ ID NO: |
| | | | | | 10 |
| PKN1 | N- | PKN1 Antibody | Novus Biologicals | Rabbit | 262-390 of |
| | term. | | (NBP1-85301) | polyclonal | SEQ ID NO: |
| | | | | IgG | 11 |
| | C- | Anti-PKN1 | LifeSpan | Rabbit | 615-874 of |
| | term. | Antibody | BioSciences, Inc. | polyclonal | SEQ ID NO: |
| | | (aa615-874) | (LS-B14604) | | 11 |
| | | IHC-plus ™ | | | |
| | | Anti-PKN1 | LifeSpan | Rabbit | 911-929 of |
| | | Antibody | BioSciences, Inc. | polyclonal | SEQ ID NO: |
| | | (aa911-929) | (LS-C147990) | | 11 |

*Immunoreactive with C-terminus of each of TrkA, TrkB, and TrkC

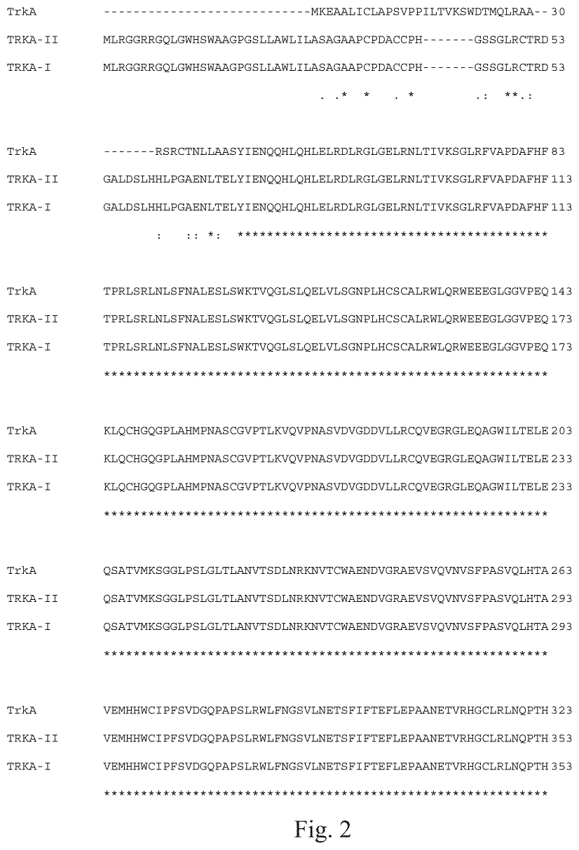

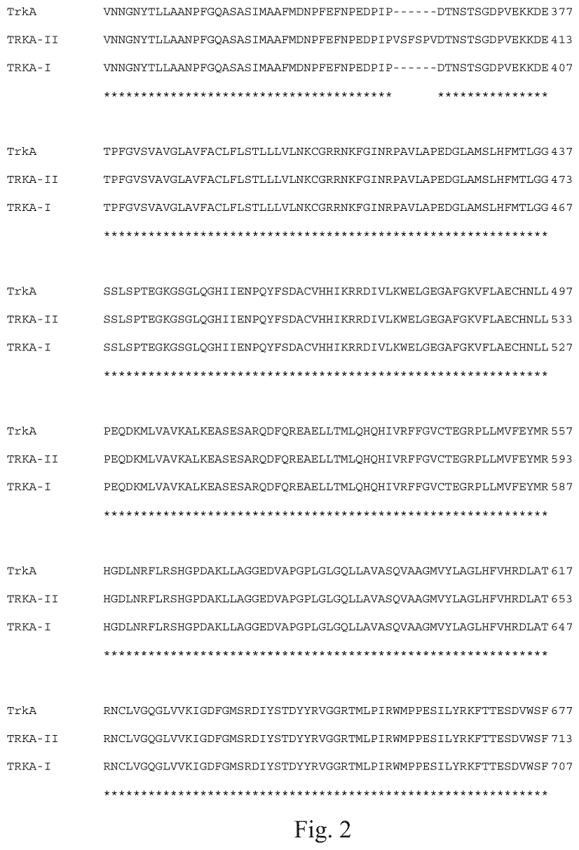

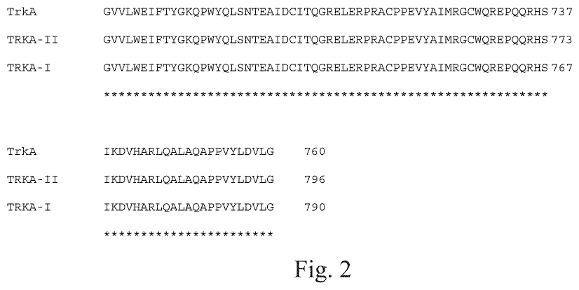

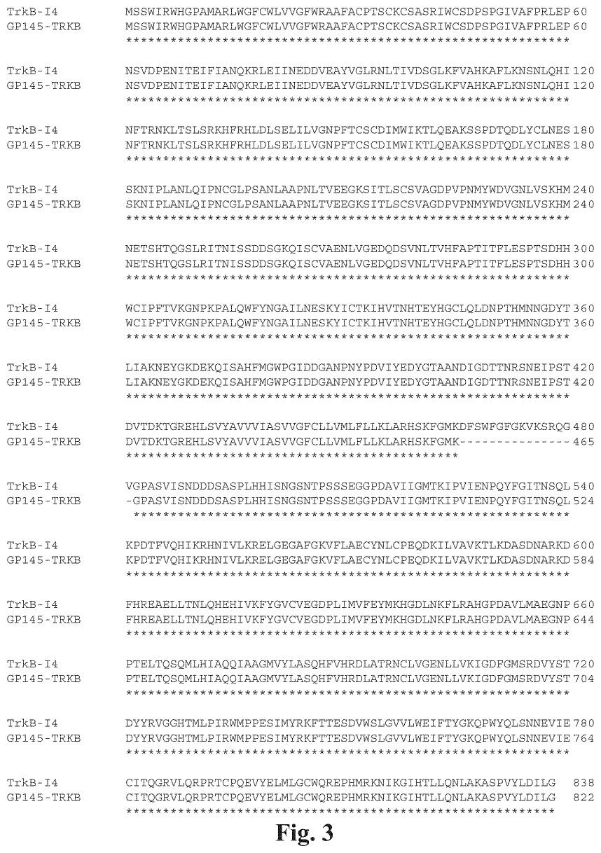

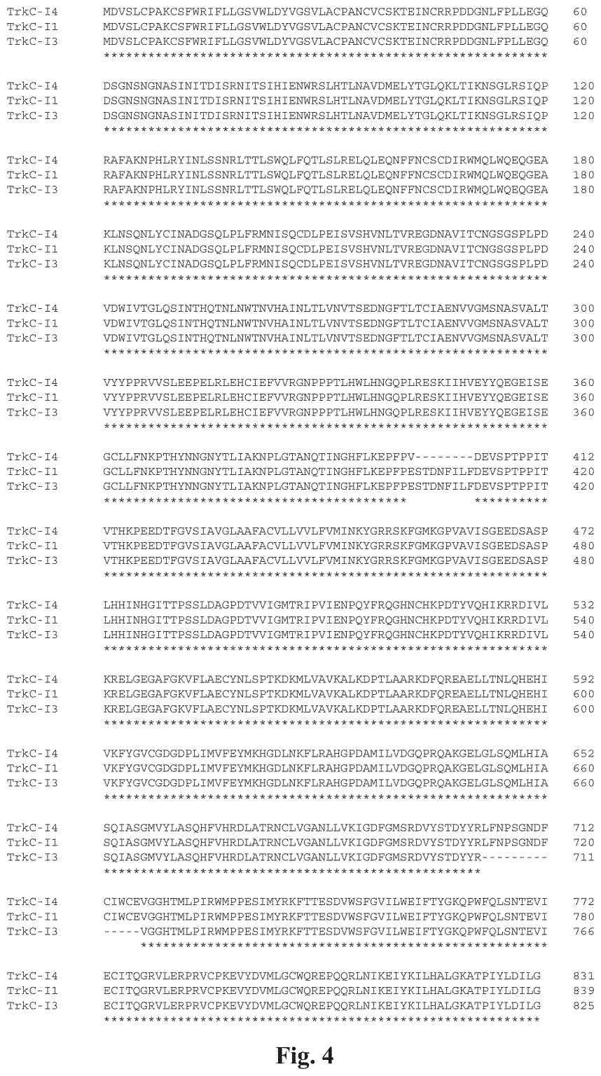

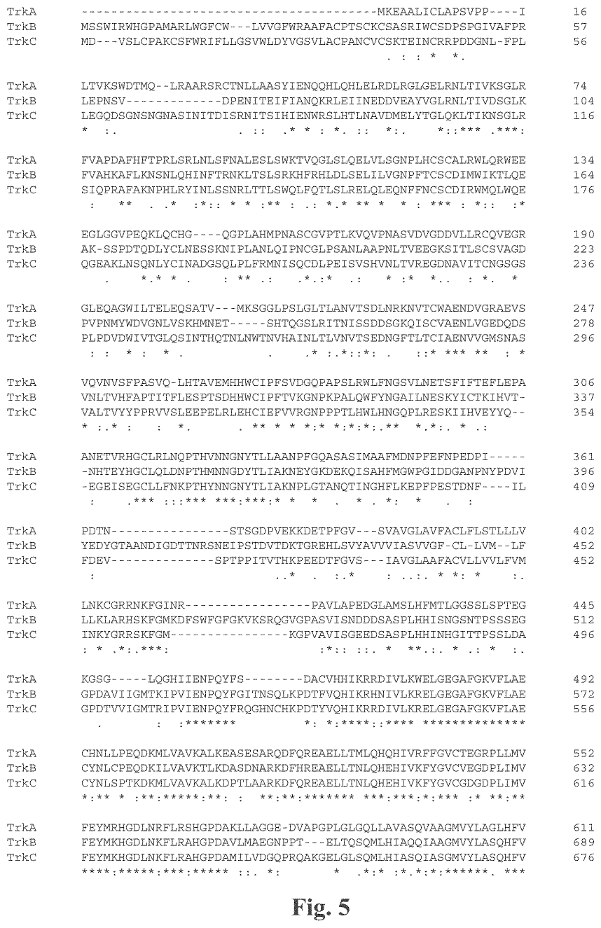

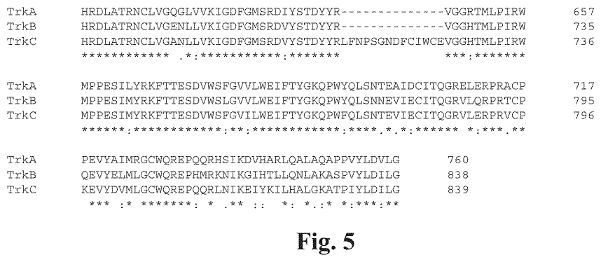

In embodiments in which the wild-type counterpart has alternate splicing variants, it may be useful to select biomarker-specific reagents that bind to the wild-type counterpart and one or more of its alternate splice variants. In such a case, the amino acid sequences of the selected splice variants are aligned with the wild-type counterpart, and non-variant regions of the wild-type counterpart are identified. The biomarker-specific reagents for the retained and lost portion or for the N- and C-terminal portion may be selected such that they are specific for a non-variant region. The one or more of its alternate splice variants in the foregoing embodiment do not need to include every known alternate splice variant of the wild-type counterpart. Rather, in some embodiments, a subset of known alternate splice variants is selected. For example, the subset may be selected as representative of the fusions found in a particular tumor type, or as representative of the fusions most likely to be encountered in clinical practice. For example, the COSMIC database reports the transcripts from which each member of a specific fusion protein is derived. For example, a defined group of alternate splice variants may be selected to represent at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or all alternate splice variants involved in fusions in a specific database (such as the COSMIC database). As another example, a defined group of alternate splice variants may be selected to represent at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or all tumors bearing a fusion protein involving a specific wild-type counterpart reported in a specific database (such as the COSMIC database). As another example, a defined group of alternate splice variants may be selected to represent at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or all tumors of a specified primary tissue type (such as lung, colorectal, breast, urinary, skin, brain, prostate, etc.) or a subtype thereof (such as non-small cell lung, small cell lung, HER+ breast, triple-negative breast, etc.) bearing a fusion protein involving a specific wild-type counterpart reported in a specific database (such as the COSMIC database). As another example, a defined group of alternate splice variants may be selected to represent at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or all tumors of a specified histological type (such as carcinoma, sarcoma, myeloma, leukemia, lymphoma, or mixed types) or a histological subtype thereof (such as adenocarcinoma, squamous cell carcinoma, osteosarcoma, chondrosarcoma, leiomyosarcoma, rhabdomyosarcoma, mesothelioma, fibrosarcoma, angiosarcoma, hemangioendothelioma, liposarcoma, glioma, astrocytoma, myxosarcoma, mesenchymous or mixed mesodermal tumor, adenosquamous carcinoma, mixed mesodermal tumor, carcinosarcoma, or teratocarcinoma) bearing a fusion protein involving a specific wild-type counterpart reported in a specific database (such as the COSMIC database). As another example, the defined group may be selected to include the top 2, top 3, top 4, top 5, etc. most prevalent alternate splice variants involved in fusion proteins reported in a specific database (such as the COSMIC database). In other embodiments, a defined group of alternate splice variants may be selected that represent at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or all alternate splice variants thereof as reported in a database (such as UNIPROT or Ensembl). For example, each of TrkA, TrkB, and TrkC have alternate splice variants. FIGS. 2-4 illustrate alignments generated using Clustal Omega (EMBL-EBI, Wellcome Genome Campus, Hinxton, Cambridgeshire) between the above-mentioned wild-type counterparts and selected major alternate splicing variants. For TrkA (FIG. 2), isoforms TrkA-I (Uniprot ID P04629-2; SEQ ID NO: 12), TrkA-II (Uniprot ID P04629-1, SEQ ID NO: 13), and TrkA Isoform 3 (Uniprot ID No. P04629-3; SEQ ID NO: 4) were aligned. TrkAIII (Uniprot ID P04629-4) was omitted from the alignment. As illustrated in FIG. 2, amino acids 42-760 of SEQ ID NO: 4 are retained in each of TrkA-I, TrkA-II, and TrkA Isoform 3, and thus considered a non-variant region of SEQ ID NO: 4 for this group of alternate splice variants. For TrkB (FIG. 3), isoforms GP145-Trkb (Uniprot ID Q16620-1; SEQ ID NO: 14) and TrkB (Isoform 4) (Uniprot ID Q16620-4; SEQ ID NO: 5) were aligned. Various C-terminal truncated isoforms were omitted from the alignment. As illustrated in FIG. 3, amino acids 1-465 and 482-838 of SEQ ID NO: 5 are retained in each isoform, and thus considered non-variant regions of SEQ ID NO: 5 for this group of alternate splice variants. For TrkC (FIG. 4), isoforms 1 (Uniprot No. Q16288-1, SEQ ID NO: 6), 3 (Uniprot No. Q16288-3, SEQ ID NO: 15), and 4 (Uniprot No. Q16288-4, SEQ ID NO: 16) were aligned. Various other isoforms were omitted from the alignment. As illustrated in FIG. 4, amino acids 1-401, 411-711, and 726-780 of SEQ ID NO: 6 are retained in each isoform, and thus considered non-variant regions of SEQ ID NO: 6 for this group of alternate splice variants.

In any of the foregoing embodiments, the biomarker-specific reagents may be any type of entity that is useful for in situ detection of proteins expressed by a cellular sample (such as by in histological or cytological staining methods). Exemplary biomarker-specific reagents include antibodies and antigen binding fragments thereof and engineered specific binding compositions, such as ADNECTINs (scaffold based on 10th FN3 fibronectin; Bristol-Myers-Squibb Co.), AFFIBODYs (scaffold based on Z domain of protein A from S. aureus; Affibody AB, Solna, Sweden), AVIMERs (scaffold based on domain A/LDL receptor; Amgen, Thousand Oaks, Calif.), dAbs (scaffold based on VH or VL antibody domain; GlaxoSmithKline PLC, Cambridge, UK), DARPins (scaffold based on Ankyrin repeat proteins; Molecular Partners A G, Zurich, C H), ANTICALINs (scaffold based on lipocalins; Pieris A G, Freising, D E), NANOBODYs (scaffold based on VHH (camelid Ig); Ablynx N/V, Ghent, B E), TRANS-BODYs (scaffold based on Transferrin; Pfizer Inc., New York, N.Y.), SMIPs (Emergent Biosolutions, Inc., Rockville, Md.), and TETRANECTINs (scaffold based on C-type lectin domain (CTLD), tetranectin; Borean Pharma A/S, Aarhus, DK). Descriptions of such engineered specific binding structures are reviewed by Wurch et al., Development of Novel Protein Scaffolds as Alternatives to Whole Antibodies for Imaging and Therapy: Status on Discovery Research and Clinical Validation, Current Pharmaceutical Biotechnology, Vol. 9, pp. 502-509 (2008), the content of which is incorporated by reference. In a specific embodiment, the first and second biomarker-specific reagents are antibodies. In another specific embodiment, the first and second antibodies are monoclonal antibodies (such as mouse monoclonal or rabbit monoclonal antibodies).

### IV. Staining Methods

In an embodiment, the sets of biomarker-specific reagents are used to stain samples suspected of harboring a fusion protein Staining is performed with the biomarker-specific reagents by affinity histochemical or affinity cytochemical methods. Affinity histochemical and cytochemical staining techniques typically involve contacting a sample deposited on a slide or other solid support with a biomarker-specific reagent under conditions sufficient to permit specific binding between the biomarker-specific reagent and the biomarker of interest. Binding of the biomarker-specific reagent to the biomarker facilitates deposition of a detectable moiety on the sample in proximity to locations containing the biomarker. The detectable moiety can be used to locate and/or quantify the biomarker to which the biomarker-specific reagent is directed. Thereby, the presence and/or concentration of the target in a sample can be detected by detecting the signal produced by the detectable moiety.

The staining process may be manual, automated, or a combination of manual and automated steps. In an embodiment, the staining process may be carried out on an automated advanced staining platform. Automated advanced staining platforms typically include at least: reservoirs of the various reagents used in the staining protocols, a reagent dispense unit in fluid communication with the reservoirs for dispensing reagent to onto a slide, a waste removal system for removing used reagents and other waste from the slide, and a control system that coordinates the actions of the reagent dispense unit and waste removal system. In addition to performing staining steps, many automated slide stainers can also perform steps ancillary to staining (or are compatible with separate systems that perform such ancillary steps), including: slide baking (for adhering the sample to the slide), dewaxing (also referred to as deparaffinization), epitope retrieval, counterstaining, dehydration and clearing, and coverslipping. Prichard describes several specific examples of automated IHC/ISH slide stainers and their various features, including the intelliPATH (Biocare Medical), WAVE (Celerus Diagnostics), DAKO OMNIS and DAKO AUTOSTAINER LINK 48 (Agilent Technologies), BENCHMARK (Ventana Medical Systems, Inc.), Leica BOND, and Lab Vision Autostainer (Thermo Scientific) automated slide stainers. Additionally, Ventana Medical Systems, Inc. is the assignee of a number of United States patents disclosing systems and methods for performing automated analyses, including U.S. Pat. Nos. 5,650,327, 5,654,200, 6,296,809, 6,352,861, 6,827,901 and 6,943,029, and U.S. Published Patent Application Nos. 20030211630 and 20040052685, each of which is incorporated herein by reference in its entirety. Commercially-available staining units typically operate on one of the following principles: (1) open individual slide staining, in which slides are positioned horizontally and reagents are dispensed as a puddle on the surface of the slide containing a tissue sample (such as implemented on the DAKO AUTOSTAINER Link 48 (Agilent Technologies) and intelliPATH (Biocare Medical) stainers); (2) liquid overlay technology, in which reagents are either covered with or dispensed through an inert fluid layer deposited over the sample (such as implemented on VENTANA BenchMark and DISCOVERY stainers); (3) capillary gap staining, in which the slide surface is placed in proximity to another surface (which may be another slide or a coverplate) to create a narrow gap, through which capillary forces draw up and keep liquid reagents in contact with the samples (such as the staining principles used by DAKO TECHMATE, Leica BOND, and DAKO OMNIS stainers). Some iterations of capillary gap staining do not mix the fluids in the gap (such as on the DAKO TECHMATE and the Leica BOND). In variations of capillary gap staining termed dynamic gap staining, capillary forces are used to apply sample to the slide, and then the parallel surfaces are translated relative to one another to agitate the reagents during incubation to effect reagent mixing (such as the staining principles implemented on DAKO OMNIS slide stainers (Agilent)). In translating gap staining, a translatable head is positioned over the slide. A lower surface of the head is spaced apart from the slide by a first gap sufficiently small to allow a meniscus of liquid to form from liquid on the slide during translation of the slide. A mixing extension having a lateral dimension less than the width of a slide extends from the lower surface of the translatable head to define a second gap smaller than the first gap between the mixing extension and the slide. During translation of the head, the lateral dimension of the mixing extension is sufficient to generate lateral movement in the liquid on the slide in a direction generally extending from the second gap to the first gap. See WO 2011-139978 A1. It has recently been proposed to use inkjet technology to deposit reagents on slides. See WO 2016-170008 A1. This list of staining technologies is not intended to be comprehensive, and any fully or semi-automated system for performing biomarker staining may be used.

### IV.A. Samples and Sample Preparation

The staining methods are practiced on cellular samples of the suspected tissue, including tissue samples and cytological samples. In some embodiments, the cellular sample is obtained from a subject having or suspected of having a tumor. In some embodiments, the sample is obtained directly from a tumor. In some embodiments, the tumor is a solid tumor, such as a carcinoma, lymphoma, or sarcoma. In an embodiment, the tumor is a tumor of the skin, breast, head and/or neck, lung, upper gastrointestinal tract (including the esophagus and stomach), female reproductive system (including uterine, fallopian, and ovarian tumors), lower gastrointestinal tract (including the colon, rectal, and anal tumors), urogenital tract, exocrine, endocrine, renal, neural, or of lymphocytic origin. In an embodiment, subject has a melanoma, breast cancer, ovarian cancer, pancreatic cancer, head and neck cancer, lung cancer, esophageal cancer, gastric cancer, colorectal cancer (including cancer of the colon, rectum, and anus), prostate, urothelial cancer, or lymphoma. In specific embodiments, the tumor is a melanoma, lung, bladder, breast, prostate, or colorectal cancer.

Where tissue samples are used, the tissue sample is processed in a manner compatible with histochemical staining, including, for example, fixation, embedding in a wax matrix (such as paraffin), and sectioning (such as with a microtome). No specific processing step is required by the present disclosure, so long as the sample obtained is compatible with histochemical staining of the sample with the set of biomarker-specific reagents. In a specific embodiment, microtome sections of formalin-fixed, paraffin-embedded (FFPE) samples are used in the staining process.

### IV.B. Epitope Retrieval and Blocking

Depending on the biomarker, the biomarker-specific reagent being used, and the sample being used, the sample may be subjected to an epitope retrieval process (also referred to as antigen retrieval) prior to application of the biomarker-specific reagent. Exemplary epitope retrieval processes include: heat-induced epitope retrieval (HIER), which involves heating the sample in various buffers at different pH levels; protease-based epitope retrieval (PBER), in which samples are digested by proteolytic enzymes prior to staining; and combinations of HIER and PBER. Various specific epitope retrieval processes are reviewed by Shi et al., D'Amico et al., Yamashita et al., Vinod et al., and Warford et al., although this is not exhaustive. Whether to perform epitope retrieval and the particular form of epitope retrieval to use depends on the specific biomarker-specific reagent selected, and may need to be empirically determined for each biomarker-specific reagent used.

Depending on the reagents and samples used, it may also be desirable to block activity of endogenous proteins prior to addition of biomarker-specific reagents and/or detection reagents. For example, where the detection reagents depend on biotin and biotin-binding proteins, it may be necessary to block endogenous biotin using, for example, free, unlabeled biotin-binding proteins. Likewise, many detection schemes rely on activity of enzymes, including phosphatases and peroxidases, which necessitates neutralization of endogenous enzymes having similar activities. Commercially-available kits are available for such blocking processes, e.g., Endogenous Biotin Blocking Kit (Cat. No. E21390, ThermoFisher Scientific), Endogenous Avidin/Biotin Blocking Kit (Cat. No. ab64212, Abcam, plc.), Endogenous Biotin Blocking Kit Cat. No. 760-050, Ventana Medical Systems, Inc.), Hydrogen Peroxide Blocking Reagent (Cat. No. ab64218, Abcam plc.), Peroxidase and Alkaline Phosphatase Blocking Reagent, (Code 52003, Agilent Technologies), among others.

It may also be useful to block sites on the sample to which the biomarker-specific reagent may bind non-specifically before applying the biomarker-specific reagent to the sample. Common blocking agents include buffered solutions of normal serum, non-fat dry milk, BSA (bovine serum albumin), and gelatin, as well as commercially available blocking agents such as eBioscience™ IHC/ICC Blocking Buffer—High Protein (Cat. No. 00-4952-54, ThermoFisher Scientific), eBioscience™ IHC/ICC Blocking Buffer—Low Protein (Cat. No. 00-4953-54, ThermoFisher Scientific), DISCOVERY antibody Block (Cat. No. 760-4204, Ventana Medical Systems, Inc.), among others.

Washing steps may be performed after each of these pre-processing steps by applying one or more passes of a wash buffer. Wash buffers typically are neutrally-buffered saline solutions, which may also contain small amounts of detergent. Exemplary wash buffers include, for example, Phosphate Buffered Saline (PBS), PBS-Tween20, Tris Buffered Saline (TBS), TBS-Tween20 (polysorbate 20), Tris-HCl, Tris-HC-Tween20, Phosphate Buffer (PB), AP Buffer, and the like.

### IV.C. Biomarker-Specific Reagent Preparation and Application

Once the sample has been prepared for staining, the biomarker-specific reagent is applied to the sample and incubated for a sufficient period of time and under conditions to promote specific binding between the biomarker and the biomarker-specific reagent.

Commercially-available biomarker-specific reagents are typically provided in a ready-to-use format or in a concentrated format. In a ready-to-use format, the biomarker-specific reagent is provided pre-diluted into a diluent at a fixed titer, which may be applied directly onto the sample. In a concentrate format, the biomarker-specific reagent must first be diluted to a working concentration in a diluent before being applied to the sample. In either case, the final working concentration of the two biomarker-specific reagents is selected to provide intensity matched staining. For example, the first and second biomarker-specific reagents are tested at various titers on serial sections of tissue samples known to express the wild-type counterpart of the fusion protein of interest. Staining obtained with the various titers of different antibodies is then compared to determine whether staining intensity is matched, and the titers of the two biomarker-specific reagents that most closely match in intensity are selected. In some embodiments, this process is repeated across a number of different samples know to have different levels of the wild-type counterpart of the fusion protein to ensure that titers for the two biomarker-specific reagents is matched across the different levels of staining that can be expected to be observed. Any method of determining titer of the biomarker-specific reagents can be used. In an embodiment, samples expressing a wild-type counterpart are stained with the biomarker-specific reagent for the lost portion at a number of different dilutions in combination with the desired detection system. The dilution giving a desired balance between sensitivity of detection and specificity of staining is selected. The process is then repeated with the biomarker-specific reagent for the retained portion, except that the selected dilution is the dilution that gives the closest match in staining profile to the biomarker-specific reagent for the lost portion. If desired, the active Ig content of the selected dilutions may be determined, for example, by enzyme-linked immunosorbent assay (ELISA), high performance liquid chromatography (HPLC), competition assay, indirect antibody assay and antigen bridging antibody assay (BA).

A washing step is typically performed after the sample is incubated with the biomarker-specific reagent by applying one or more passes of a wash buffer. This removes unbound or non-specifically bound biomarker-specific reagent from the sample to mitigate off-target and/or background staining.

### IV.D. Labeling Schemes and Associated Reagents

Detection of the biomarker in the sample is achieved by depositing a detectable moiety in close proximity to the biomarker-specific reagent bound to the sample. In some embodiments, the detectable moiety is directly conjugated to the biomarker-specific reagent, and thus is deposited on the sample upon binding of the biomarker-specific reagent to its target (generally referred to as a direct labeling method). In other embodiments, deposition of the detectable moiety is effected by the applying a set of detection reagents to the sample after the application of the biomarker-specific reagent, wherein the detection reagents bind to or otherwise react with the biomarker-specific reagent in a manner the effects deposition of the detectable moiety (generally referred to as an indirect labeling method).

In some embodiments in which an indirect method is used, the detectable moiety is deposited via an enzymatic reaction localized to the biomarker-specific reagent. Suitable enzymes for such reactions are well-known and include, but are not limited to, oxidoreductases, hydrolases, and peroxidases. Specific enzymes explicitly included are horseradish peroxidase (HRP), alkaline phosphatase (AP), acid phosphatase, glucose oxidase, β-galactosidase, β-glucuronidase, and β-lactamase. The enzyme may be directly conjugated to the biomarker-specific reagent, or may be indirectly associated with the biomarker-specific reagent via a labeling conjugate. As used herein, a “labeling conjugate” comprises:
(a) a specific detection reagent; and
(b) an enzyme conjugated to the specific detection reagent, wherein the enzyme is reactive with a chromogenic substrate, a signaling conjugate, and/or an enzyme-reactive dye under appropriate reaction conditions to effect in situ generation of the dye and/or deposition of the dye on the tissue sample.

In non-limiting examples, the specific detection reagent of the labeling conjugate may be a secondary detection reagent (such as a species-specific secondary antibody bound to a primary antibody, an anti-hapten antibody bound to a hapten-conjugated primary antibody, or a biotin-binding protein bound to a biotinylated primary antibody), a tertiary detection reagent (such as a species-specific tertiary antibody bound to a secondary antibody, an anti-hapten antibody bound to a hapten-conjugated secondary antibody, or a biotin-binding protein bound to a biotinylated secondary antibody), or other such arrangements. An enzyme thus localized to the sample-bound biomarker-specific reagent can then be used in a number of schemes to deposit a detectable moiety.

In some cases, the enzyme reacts with a chromogenic compound/substrate. Particular non-limiting examples of chromogenic compounds/substrates include 4-nitrophenylphospate (pNPP), fast red, bromochloroindolyl phosphate (BCIP), nitro blue tetrazolium (NBT), BCIP/NBT, fast red, AP Orange, AP blue, tetramethylbenzidine (TMB), 2,2′-azino-di-[3-ethylbenzothiazoline sulphonate] (ABTS), o-dianisidine, 4-chloronaphthol (4-CN), nitrophenyl-β-D-galactopyranoside (ONPG), o-phenylenediamine (OPD), 5-bromo-4-chloro-3-indolyl-β-galactopyranoside (X-Gal), methylumbelliferyl-β-D-galactopyranoside (MU-Gal), p-nitrophenyl-α-D-galactopyranoside (PNP), 5-bromo-4-chloro-3-indolyl-β-D-glucuronide (X-Gluc), 3-amino-9-ethyl carbazol (AEC), fuchsin, iodonitrotetrazolium (INT), tetrazolium blue, or tetrazolium violet.

In some embodiments, the enzyme can be used in a metallographic detection scheme. Metallographic detection methods include using an enzyme such as alkaline phosphatase in combination with a water-soluble metal ion and a redox-inactive substrate of the enzyme. In some embodiments, the substrate is converted to a redox-active agent by the enzyme, and the redox-active agent reduces the metal ion, causing it to form a detectable precipitate. (see, for example, U.S. patent application Ser. No. 11/015,646, filed Dec. 20, 2004, PCT Publication No. 2005/003777 and U.S. Patent Application Publication No. 2004/0265922; each of which is incorporated by reference herein in its entirety). Metallographic detection methods include using an oxido-reductase enzyme (such as horseradish peroxidase) along with a water soluble metal ion, an oxidizing agent and a reducing agent, again to for form a detectable precipitate. (See, for example, U.S. Pat. No. 6,670,113, which is incorporated by reference herein in its entirety).

In some embodiments, the enzymatic action occurs between the enzyme and the dye itself, wherein the reaction converts the dye from a non-binding species to a species deposited on the sample. For example, reaction of DAB with a peroxidase (such as horseradish peroxidase) oxidizes the DAB, causing it to precipitate.

In yet other embodiments, the detectable moiety is deposited via a signaling conjugate comprising a latent reactive moiety configured to react with the enzyme to form a reactive species that can bind to the sample or to other detection components. These reactive species are capable of reacting with the sample proximal to their generation, i.e. near the enzyme, but rapidly convert to a non-reactive species so that the signaling conjugate is not deposited at sites distal from the site at which the enzyme is deposited. Examples of latent reactive moieties include: quinone methide (QM) analogs, such as those described at WO2015124703A1, and tyramide conjugates, such as those described at, WO2012003476A2, each of which is hereby incorporated by reference herein in its entirety. In some examples, the latent reactive moiety is directly conjugated to a dye, such as N,N′-biscarboxypentyl-5,5 ‘-disulfonato-indo-dicarbocyanine (Cy5), 4-(dimethylamino) azobenzene-4’-sulfonamide (DABSYL), tetramethylrhodamine (DISCO Purple), and Rhodamine 110 (Rhodamine). In other examples, the latent reactive moiety is conjugated to one member of a specific binding pair, and the dye is linked to the other member of the specific binding pair. In other examples, the latent reactive moiety is linked to one member of a specific binding pair, and an enzyme is linked to the other member of the specific binding pair, wherein the enzyme is (a) reactive with a chromogenic substrate to effect generation of the dye, or (b) reactive with a dye to effect deposition of the dye (such as DAB). Examples of specific binding pairs include:
(1) a biotin or a biotin derivative (such as desthiobiotin) linked to the latent reactive moiety, and a biotin-binding entity (such as avidin, streptavidin, deglycosylated avidin (such as NEUTRAVIDIN), or a biotin binding protein having a nitrated tyrosine at its biotin binding site (such as CAPTAVIDIN)) linked to a dye or to an enzyme reactive with a chromogenic substrate or reactive with a dye (for example, a peroxidase linked to the biotin-binding protein when the dye is DAB); and
(2) a hapten linked to the latent reactive moiety, and an anti-hapten antibody linked to a dye or to an enzyme reactive with a chromogenic substrate or reactive with a dye (for example, a peroxidase linked to the biotin-binding protein when the dye is DAB).
   Non-limiting examples of biomarker-specific reagent and detection reagent combinations are set forth in Table 6 are specifically included.

TABLE 6
A. Biomarker-specific reagent linked directly to detectable moiety
Biomarker-specific reagent-Dye conjugate
B. Biomarker-specific reagent linked to enzyme reacting with detectable moiety
Biomarker-specific reagent-Enzyme conjugate + DAB
Biomarker-specific reagent-Enzyme conjugate + Chromogen
Biomarker-specific reagent-Enzyme conjugate + Fluorophore
C. Biomarker-specific reagent linked to Enzyme reacting with detectable moiety

| | |
|---|---|
| C1. Signaling conjugate | Biomarker-specific reagent-Enzyme conjugate + QM-Dye |
| comprises detectable moiety | conjugate |
| | Biomarker-specific reagent-Enzyme conjugate + Tyramide- |
| | Dye conjugate |
| C2. Signaling conjugate | Biomarker-specific reagent-Enzyme conjugate + QM- |
| comprises enzyme that reacts | Enzyme conjugate + DAB |
| directly with detectable | Biomarker-specific reagent-Enzyme conjugate + QM- |
| moiety | Enzyme conjugate + Chromogen |
| | Biomarker-specific reagent-Enzyme conjugate + QM- |
| | Enzyme conjugate + Fluorophore |
| | Biomarker-specific reagent-Enzyme conjugate + Tyramide- |
| | Enzyme conjugate + DAB |
| | Biomarker-specific reagent-Enzyme conjugate + Tyramide- |
| | Enzyme conjugate + Chromogen |
| | Biomarker-specific reagent-Enzyme conjugate + Tyramide- |
| | Enzyme conjugate + Fluorophore |
| C3. Signaling conjugate | Biomarker-specific reagent-Enzyme conjugate + QM- |
| comprises enzyme that reacts | Enzyme conjugate + QM-Dye conjugate |
| with second signaling | Biomarker-specific reagent-Enzyme conjugate + QM- |
| conjugate comprising | Enzyme conjugate + Tyramide-Dye conjugate |
| detectable moiety | Biomarker-specific reagent-Enzyme conjugate + Tyramide- |
| | Enzyme conjugate + QM-Dye conjugate |
| | Biomarker-specific reagent-Enzyme conjugate + Tyramide- |
| | Enzyme conjugate + Tyramide-Dye conjugate |
| C4. Signaling conjugate | Biomarker-specific reagent-Enzyme conjugate + Tyramide- |
| comprises member of a | (biotin or hapten) conjugate + Dye-(avidin or anti-hapten |
| specific binding pair and | specific detection reagent) conjugate |
| other member of binding pair | Biomarker-specific reagent-Enzyme conjugate + QM-(biotin |
| is linked to detectable moiety | or hapten) conjugate + Dye-(avidin or anti-hapten specific |
| | detection reagent) conjugate |
| C5. Signaling conjugate | Biomarker-specific reagent-Enzyme conjugate + QM-(biotin |
| comprises member of a | or hapten) conjugate + Enzyme-(avidin or anti-hapten |
| specific binding pair and | specific detection reagent) conjugate + DAB |
| other member of binding pair | Biomarker-specific reagent-Enzyme conjugate + QM-(biotin |
| is linked to enzyme reactive | or hapten) conjugate + Enzyme-(avidin or anti-hapten |
| with detectable moiety | specific detection reagent) conjugate + Chromogen |
| | Biomarker-specific reagent-Enzyme conjugate + QM-(biotin |
| | or hapten) conjugate + Enzyme-(avidin or anti-hapten |
| | specific detection reagent) conjugate + Fluorophore |
| | Biomarker-specific reagent-Enzyme conjugate + Tyramide- |
| | (biotin or hapten) conjugate + Enzyme-(avidin or anti-hapten |
| | specific detection reagent) conjugate + DAB |
| | Biomarker-specific reagent-Enzyme conjugate + Tyramide- |
| | (biotin or hapten) conjugate + Enzyme-(avidin or anti-hapten |
| | specific detection reagent) conjugate + Chromogen |
| | Biomarker-specific reagent-Enzyme conjugate + Tyramide- |
| | (biotin or hapten) conjugate + Enzyme-(avidin or anti-hapten |
| | specific detection reagent) conjugate + Fluorophore |
| C6. Signaling conjugate | Biomarker-specific reagent-Enzyme conjugate + QM-(biotin |
| comprises member of a | or hapten) conjugate + Enzyme-(avidin or anti-hapten |
| specific binding pair and | specific detection reagent) conjugate + Tyramide-Dye |
| other member of binding pair | conjugate |
| is linked to enzyme reactive | Biomarker-specific reagent-Enzyme conjugate + QM-(biotin |
| with second detectable | or hapten) conjugate + Enzyme-(avidin or anti-hapten |
| moiety linked to a detectable | specific detection reagent) conjugate + QM-Dye conjugate |
| moiety | Biomarker-specific reagent-Enzyme conjugate + Tyramide- |
| | (biotin or hapten) conjugate + Enzyme-(avidin or anti-hapten |
| | specific detection reagent) conjugate + Tyramide-Dye |
| | conjugate |
| | Biomarker-specific reagent-Enzyme conjugate + Tyramide- |
| | (biotin or hapten) conjugate + Enzyme-(avidin or anti-hapten |
| | specific detection reagent) conjugate + QM-Dye conjugate |

D. Biomarker-specific reagent linked to member of specific binding pair

| | |
|---|---|
| D1. Dye linked to other | Biomarker-specific reagent-(biotin or hapten) conjugate + |
| member of specific binding | Dye-(avidin or anti-hapten specific detection reagent) |
| pair | conjugate |
| D2. Enzyme linked to other | Biomarker-specific reagent-(biotin or hapten) conjugate + |
| member of specific binding | Enzyme-(avidin or anti-hapten specific detection reagent) |
| pair, wherein the enzyme is | conjugate + DAB |
| reactive with detectable | Biomarker-specific reagent-(biotin or hapten) conjugate + |
| moiety | Enzyme-(avidin or anti-hapten specific detection reagent) |
| | conjugate + Chromogen |
| | Biomarker-specific reagent-(biotin or hapten) conjugate + |
| | Enzyme-(avidin or anti-hapten specific detection reagent) |
| | conjugate + fluorophore |
| | Biomarker-specific reagent-(biotin or hapten) conjugate + |
| | Enzyme-(avidin or anti-hapten specific detection reagent) |
| | conjugate + QM-Dye conjugate |
| | Biomarker-specific reagent-(biotin or hapten) conjugate + |
| | Enzyme-(avidin or anti-hapten specific detection reagent) |
| | conjugate + Tyramide-Dye conjugate |

E. Secondary detection reagent linked directly to detectable moiety
Biomarker-specific reagent + 2° specific detection reagent-Dye conjugate
F. Secondary detection reagent linked to Enzyme reacting with detectable moiety
Biomarker-specific reagent + 2° specific detection reagent-Enzyme conjugate + DAB
Biomarker-specific reagent + 2° specific detection reagent-Enzyme conjugate + Chromogen
Biomarker-specific reagent + 2° specific detection reagent-Enzyme conjugate + Fluorophore
G. Secondary detection reagent linked to Enzyme reacting with detectable moiety

| | |
|---|---|
| G1. Signaling conjugate | Biomarker-specific reagent + 2° specific detection reagent- |
| comprises detectable moiety | Enzyme conjugate + QM-Dye conjugate |
| | Biomarker-specific reagent + 2° specific detection reagent- |
| | Enzyme conjugate + Tyramide-Dye conjugate |
| G2. Signaling conjugate | Biomarker-specific reagent + 2° specific detection reagent- |
| comprises enzyme that reacts | Enzyme conjugate + QM-Enzyme conjugate + DAB |
| directly with detectable | Biomarker-specific reagent + 2° specific detection reagent- |
| moiety | Enzyme conjugate + QM-Enzyme conjugate + Chromogen |
| | Biomarker-specific reagent + 2° specific detection reagent- |
| | Enzyme conjugate + QM-Enzyme conjugate + Fluorophore |
| | Biomarker-specific reagent + 2° specific detection reagent- |
| | Enzyme conjugate + Tyramide-Enzyme conjugate + DAB |
| | Biomarker-specific reagent + 2° specific detection reagent- |
| | Enzyme conjugate + Tyramide-Enzyme conjugate + |
| | Chromogen |
| | Biomarker-specific reagent + 2° specific detection reagent- |
| | Enzyme conjugate + Tyramide-Enzyme conjugate + |
| | Fluorophore |
| G3. Signaling conjugate | Biomarker-specific reagent + 2° specific detection reagent- |
| comprises enzyme that reacts | Enzyme conjugate + QM-Enzyme conjugate + QM-Dye |
| with second signaling | conjugate |
| conjugate comprising | Biomarker-specific reagent + 2° specific detection reagent- |
| detectable moiety | Enzyme conjugate + QM-Enzyme conjugate + Tyramide- |
| | Dye conjugate |
| | Biomarker-specific reagent + 2° specific detection reagent- |
| | Enzyme conjugate + Tyramide-Enzyme conjugate + QM- |
| | Dye conjugate |
| | Biomarker-specific reagent + 2° specific detection reagent- |
| | Enzyme conjugate + Tyramide-Enzyme conjugate + |
| | Tyramide-Dye conjugate |
| G4. Signaling conjugate | Biomarker-specific reagent + 2° specific detection reagent- |
| comprises member of a | Enzyme conjugate + Tyramide-(biotin or hapten) conjugate + |
| specific binding pair and | Dye-(avidin or anti-hapten specific detection reagent) |
| other member of binding pair | conjugate |
| is linked to detectable moiety | Biomarker-specific reagent + 2° specific detection reagent- |
| | Enzyme conjugate + QM-(biotin or hapten) conjugate + Dye- |
| | (avidin or anti-hapten specific detection reagent) conjugate |
| G5. Signaling conjugate | Biomarker-specific reagent + 2° specific detection reagent- |
| comprises member of a | Enzyme conjugate + QM-(biotin or hapten) conjugate + |
| specific binding pair and | Enzyme-(avidin or anti-hapten specific detection reagent) |
| other member of binding pair | conjugate + DAB |
| is linked to enzyme reactive | Biomarker-specific reagent + 2° specific detection reagent- |
| with detectable moiety | Enzyme conjugate + QM-(biotin or hapten) conjugate + |
| | Enzyme-(avidin or anti-hapten specific detection reagent) |
| | conjugate + Chromogen |
| | Biomarker-specific reagent + 2° specific detection reagent- |
| | Enzyme conjugate + QM-(biotin or hapten) conjugate + |
| | Enzyme-(avidin or anti-hapten specific detection reagent) |
| | conjugate + Fluorophore |
| | Biomarker-specific reagent + 2° specific detection reagent- |
| | Enzyme conjugate + Tyramide-(biotin or hapten) conjugate + |
| | Enzyme-(avidin or anti-hapten specific detection reagent) |
| | conjugate + DAB |
| | Biomarker-specific reagent + 2° specific detection reagent- |
| | Enzyme conjugate + Tyramide-(biotin or hapten) conjugate + |
| | Enzyme-(avidin or anti-hapten specific detection reagent) |
| | conjugate + Chromogen |
| | Biomarker-specific reagent + 2° specific detection reagent- |
| | Enzyme conjugate + Tyramide-(biotin or hapten) conjugate + |
| | Enzyme-(avidin or anti-hapten specific detection reagent) |
| | conjugate + Flurophore |
| G6. Signaling conjugate | Biomarker-specific reagent + 2° specific detection reagent- |
| comprises member of a | Enzyme conjugate + QM-(biotin or hapten) conjugate + |
| specific binding pair and | Enzyme-(avidin or anti-hapten specific detection reagent) |
| other member of binding pair | conjugate + Tyramide-Dye conjugate |
| is linked to enzyme reactive | Biomarker-specific reagent + 2° specific detection reagent- |
| with second detectable | Enzyme conjugate + QM-(biotin or hapten) conjugate + |
| moiety linked to a detectable | Enzyme-(avidin or anti-hapten specific detection reagent) |
| moiety | conjugate + QM-Dye conjugate |
| | Biomarker-specific reagent + 2° specific detection reagent- |
| | Enzyme conjugate + Tyramide-(biotin or hapten) conjugate + |
| | Enzyme-(avidin or anti-hapten specific detection reagent) |
| | conjugate + Tyramide-Dye conjugate |
| | Biomarker-specific reagent + 2° specific detection reagent- |
| | Enzyme conjugate + Tyramide-(biotin or hapten) conjugate + |
| | Enzyme-(avidin or anti-hapten specific detection reagent) |
| | conjugate + QM-Dye conjugate |

H. Secondary detection reagent linked to member of specific binding pair

| | |
|---|---|
| H1. Dye linked to other | Biomarker-specific reagent + 2° specific detection reagent- |
| member of specific binding | (biotin or hapten) conjugate + Dye-(avidin or anti-hapten |
| pair | specific detection reagent) conjugate |
| H2. Enzyme linked to | Biomarker-specific reagent + 2° specific detection reagent- |
| other member of specific | (biotin or hapten) conjugate + Enzyme-(avidin or anti-hapten |
| binding pair, wherein the | specific detection reagent) conjugate + DAB |
| enzyme is reactive with | Biomarker-specific reagent + 2° specific detection reagent- |
| detectable moiety | (biotin or hapten) conjugate + Enzyme-(avidin or anti-hapten |
| | specific detection reagent) conjugate + Chromogen |
| | Biomarker-specific reagent + 2° specific detection reagent- |
| | (biotin or hapten) conjugate + Enzyme-(avidin or anti-hapten |
| | specific detection reagent) conjugate + Fluorophore |
| | Biomarker-specific reagent + 2° specific detection reagent- |
| | (biotin or hapten) conjugate + Enzyme-(avidin or anti-hapten |
| | specific detection reagent) conjugate + QM-Dye conjugate |
| | Biomarker-specific reagent + 2° specific detection reagent- |
| | (biotin or hapten) conjugate + Enzyme-(avidin or anti-hapten |
| | specific detection reagent) conjugate + Tyramide-Dye |
| | conjugate |

I. Tertiary specific detection reagent linked directly to detectable moiety
Biomarker-specific reagent + 2° specific detection reagent + 3° specific detection reagent-
Dye conjugate
J. Tertiary specific detection reagent linked to Enzyme reacting with detectable
moiety
Biomarker-specific reagent + 2° specific detection reagent + 3° specific detection reagent-
Enzyme conjugate + DAB
Biomarker-specific reagent + 2° specific detection reagent + 3° specific detection reagent-
Enzyme conjugate + Chromogen
Biomarker-specific reagent + 2° specific detection reagent + 3° specific detection reagent-
Enzyme conjugate + Fluorophore
K. Tertiary specific detection reagent linked to Enzyme reacting with detectable
moiety

| | |
|---|---|
| K1. Signaling conjugate | Biomarker-specific reagent + 2° specific detection reagent + |
| comprises detectable moiety | 3° specific detection reagent-Enzyme conjugate + QM-Dye |
| | conjugate |
| | Biomarker-specific reagent + 2° specific detection reagent + |
| | 3° specific detection reagent-Enzyme conjugate + Tyramide- |
| | Dye conjugate |
| K2. Signaling conjugate | Biomarker-specific reagent + 2° specific detection reagent + |
| comprises enzyme that reacts | 3° specific detection reagent-Enzyme conjugate + QM- |
| directly with detectable | Enzyme conjugate + DAB |
| moiety | Biomarker-specific reagent + 2° specific detection reagent + |
| | 3° specific detection reagent-Enzyme conjugate + QM- |
| | Enzyme conjugate + Chromogen |
| | Biomarker-specific reagent + 2° specific detection reagent + |
| | 3° specific detection reagent-Enzyme conjugate + QM- |
| | Enzyme conjugate + Fluorophore |
| | Biomarker-specific reagent + 2° specific detection reagent + |
| | 3° specific detection reagent-Enzyme conjugate + Tyramide- |
| | Enzyme conjugate + DAB |
| | Biomarker-specific reagent + 2° specific detection reagent + |
| | 3° specific detection reagent-Enzyme conjugate + Tyramide- |
| | Enzyme conjugate + Chromogen |
| | Biomarker-specific reagent + 2° specific detection reagent + |
| | 3° specific detection reagent-Enzyme conjugate + Tyramide- |
| | Enzyme conjugate + Fluorophore |
| K3. Signaling conjugate | Biomarker-specific reagent + 2° specific detection reagent + |
| comprises enzyme that reacts | 3° specific detection reagent-Enzyme conjugate + QM- |
| with second signaling | Enzyme conjugate + QM-Dye conjugate |
| conjugate comprising | Biomarker-specific reagent + 2° specific detection reagent + |
| detectable moiety | 3° specific detection reagent-Enzyme conjugate + QM- |
| | Enzyme conjugate + Tyramide-Dye conjugate |
| | Biomarker-specific reagent + 2° specific detection reagent + |
| | 3° specific detection reagent-Enzyme conjugate + Tyramide- |
| | Enzyme conjugate + QM-Dye conjugate |
| | Biomarker-specific reagent + 2° specific detection reagent + |
| | 3° specific detection reagent-Enzyme conjugate + Tyramide- |
| | Enzyme conjugate + Tyramide-Dye conjugate |
| K4. Signaling conjugate | Biomarker-specific reagent + 2° specific detection reagent + |
| comprises member of a | 3° specific detection reagent-Enzyme conjugate + Tyramide- |
| specific binding pair and | (biotin or hapten) conjugate + Dye-(avidin or anti-hapten |
| other member of binding pair | specific detection reagent) conjugate |
| is linked to detectable moiety | Biomarker-specific reagent + 2° specific detection reagent + |
| | 3° specific detection reagent-Enzyme conjugate + QM- |
| | (biotin or hapten) conjugate + Dye-(avidin or anti-hapten |
| | specific detection reagent) conjugate |
| K5. Signaling conjugate | Biomarker-specific reagent + 2° specific detection reagent + |
| comprises member of a | 3° specific detection reagent-Enzyme conjugate + QM- |
| specific binding pair and | (biotin or hapten) conjugate + Enzyme-(avidin or anti-hapten |
| other member of binding pair | specific detection reagent) conjugate + DAB |
| is linked to enzyme reactive | Biomarker-specific reagent + 2° specific detection reagent + |
| with detectable moiety | 3° specific detection reagent-Enzyme conjugate + QM- |
| | (biotin or hapten) conjugate + Enzyme-(avidin or anti-hapten |
| | specific detection reagent) conjugate + Chromogen |
| | Biomarker-specific reagent + 2° specific detection reagent + |
| | 3° specific detection reagent-Enzyme conjugate + QM- |
| | (biotin or hapten) conjugate + Enzyme-(avidin or anti-hapten |
| | specific detection reagent) conjugate + Fluorophore |
| | Biomarker-specific reagent + 2° specific detection reagent + |
| | 3° specific detection reagent-Enzyme conjugate + Tyramide- |
| | (biotin or hapten) conjugate + Enzyme-(avidin or anti-hapten |
| | specific detection reagent) conjugate + DAB |
| | Biomarker-specific reagent + 2° specific detection reagent + |
| | 3° specific detection reagent-Enzyme conjugate + Tyramide- |
| | (biotin or hapten) conjugate + Enzyme-(avidin or anti-hapten |
| | specific detection reagent) conjugate + Chromogen |
| | Biomarker-specific reagent + 2° specific detection reagent + |
| | 3° specific detection reagent-Enzyme conjugate + Tyramide- |
| | (biotin or hapten) conjugate + Enzyme-(avidin or anti-hapten |
| | specific detection reagent) conjugate + Fluorophore |
| K6. Signaling conjugate | Biomarker-specific reagent + 2° specific detection reagent + |
| comprises member of a | 3° specific detection reagent-Enzyme conjugate + QM- |
| specific binding pair and | (biotin or hapten) conjugate + Enzyme-(avidin or anti-hapten |
| other member of binding pair | specific detection reagent) conjugate + Tyramide-Dye |
| is linked to enzyme reactive | conjugate |
| with second detectable | Biomarker-specific reagent + 2° specific detection reagent + |
| moiety linked to a detectable | 3° specific detection reagent-Enzyme conjugate + QM- |
| moiety | (biotin or hapten) conjugate + Enzyme-(avidin or anti-hapten |
| | specific detection reagent) conjugate + QM-Dye conjugate |
| | Biomarker-specific reagent + 2° specific detection reagent + |
| | 3° specific detection reagent-Enzyme conjugate + Tyramide- |
| | (biotin or hapten) conjugate + Enzyme-(avidin or anti-hapten |
| | specific detection reagent) conjugate + Tyramide-Dye |
| | conjugate |
| | Biomarker-specific reagent + 2° specific detection reagent + |
| | 3° specific detection reagent-Enzyme conjugate + Tyramide- |
| | (biotin or hapten) conjugate + Enzyme-(avidin or anti-hapten |
| | specific detection reagent) conjugate + QM-Dye conjugate |

L. Tertiary specific detection reagent linked to member of specific binding pair

| | |
|---|---|
| L1. Dye linked to other | Biomarker-specific reagent + 2° specific detection reagent + |
| member of specific binding | 3° specific detection reagent-(biotin or hapten) conjugate + |
| pair | Dye-(avidin or anti-hapten specific detection reagent) |
| | conjugate |
| L2. Enzyme linked to | Biomarker-specific reagent + 2° specific detection reagent + |
| other member of specific | 3° specific detection reagent-(biotin or hapten) conjugate + |
| binding pair, wherein the | Enzyme-(avidin or anti-hapten specific detection reagent) |
| enzyme is reactive with | conjugate + DAB |
| detectable moiety | Biomarker-specific reagent + 2° specific detection reagent + |
| | 3° specific detection reagent-(biotin or hapten) conjugate + |
| | Enzyme-(avidin or anti-hapten specific detection reagent) |
| | conjugate + Chromogen |
| | Biomarker-specific reagent + 2° specific detection reagent + |
| | 3° specific detection reagent-(biotin or hapten) conjugate + |
| | Enzyme-(avidin or anti-hapten specific detection reagent) |
| | conjugate + Fluorophore |
| | Biomarker-specific reagent + 2° specific detection reagent + |
| | 3° specific detection reagent-(biotin or hapten) conjugate + |
| | Enzyme-(avidin or anti-hapten specific detection reagent) |
| | conjugate + QM-Dye conjugate |
| | Biomarker-specific reagent + 2° specific detection reagent + |
| | 3° specific detection reagent-(biotin or hapten) conjugate + |
| | Enzyme-(avidin or anti-hapten specific detection reagent) |
| | conjugate + Tyramide-Dye conjugate |

In a specific embodiment, the biomarker-specific reagents and the specific detection reagents set forth in Table 6 are antibodies. As would be appreciated by a person having ordinary skill in the art, the detection scheme for each of the biomarker-specific reagent may be the same, or it may be different.
Non-limiting examples of commercially available detection reagents or kits comprising detection reagents suitable for use with present methods include: VENTANA ultraView detection systems (secondary antibodies conjugated to enzymes, including HRP and AP); VENTANA iVIEW detection systems (biotinylated anti-species secondary antibodies and streptavidin-conjugated enzymes); VENTANA OptiView detection systems (OptiView) (anti-species secondary antibody conjugated to a hapten and an anti-hapten tertiary antibody conjugated to an enzyme multimer); VENTANA Amplification kit (unconjugated secondary antibodies, which can be used with any of the foregoing VENTANA detection systems to amplify the number of enzymes deposited at the site of primary antibody binding); VENTANA OptiView Amplification system (Anti-species secondary antibody conjugated to a hapten, an anti-hapten tertiary antibody conjugated to an enzyme multimer, and a tyramide conjugated to the same hapten. In use, the secondary antibody is contacted with the sample to effect binding to the primary antibody. Then the sample is incubated with the anti-hapten antibody to effect association of the enzyme to the secondary antibody. The sample is then incubated with the tyramide to effect deposition of additional hapten molecules. The sample is then incubated again with the anti-hapten antibody to effect deposition of additional enzyme molecules. The sample is then incubated with the detectable moiety to effect dye deposition); VENTANA DISCOVERY, DISCOVERY OmniMap, DISCOVERY UltraMap anti-hapten antibody, secondary antibody, chromogen, fluorophore, and dye kits, each of which are available from Ventana Medical Systems, Inc. (Tucson, Ariz.); PowerVision and PowerVision+ IHC Detection Systems (secondary antibodies directly polymerized with HRP or AP into compact polymers bearing a high ratio of enzymes to antibodies); and DAKO EnVision™+ System (enzyme labeled polymer that is conjugated to secondary antibodies).

### IV.E. Counterstaining

If desired, the biomarker-stained slides may be counterstained to assist in identifying morphologically relevant areas. Examples of counterstains include chromogenic nuclear counterstains, such as hematoxylin (stains from blue to violet), Methylene blue (stains blue), toluidine blue (stains nuclei deep blue and polysaccharides pink to red), nuclear fast red (also called Kernechtrot dye, stains red), and methyl green (stains green); non-nuclear chromogenic stains, such as eosin (stains pink); fluorescent nuclear stains, including 4′, 6-diamino-2-pheylindole (DAPI, stains blue), propidium iodide (stains red), Hoechst stain (stains blue), nuclear green DCS1 (stains green), nuclear yellow (Hoechst 5769121, stains yellow under neutral pH and stains blue under acidic pH), DRAQ5 (stains red), DRAQ7 (stains red); fluorescent non-nuclear stains, such as fluorophore-labelled phalloidin, (stains filamentous actin, color depends on conjugated fluorophore).

### IV.F. Morphological Staining of Samples

In certain embodiments, it may also be desirable to morphologically stain a portion of the sample. For example, where the 1″ and 2^{nd} biomarker-specific reagents are used to stain serial sections of a tissue sample, an additional serial section may be morphologically stained for, e.g., primary diagnosis, identification of regions of interest for digital analysis (if desired), etc.

Basic morpohological staining techniques often rely on staining nuclear structures with a first dye, and staining cytoplasmic structures with a second stain. Many morphological stains are known, including but not limited to, hematoxylin and eosin (H&E) stain and Lee's Stain (Methylene Blue and Basic Fuchsin). In a specific embodiment, at least one serial section of each biomarker-stained slide is H&E stained. Any method of applying H&E stain may be used, including manual and automated methods. In an embodiment, at least one section of the sample is an H&E stained sampled stained on an automated H&E staining system. Automated H&E systems typically operate on one of two staining principles: batch staining (also referred to as “dip ‘n dunk”) or individual slide staining. Batch stainers generally use vats or baths of reagents in which many slides are immersed at the same time. Individual slide stainers, on the other hand, apply reagent directly to each slide, and no two slides share the same aliquot of reagent. Examples of commercially available H&E stainers include the VENTANA SYMPHONY (individual slide stainer) and VENTANA HE 600 (individual slide stainer) series H&E stainers from Roche; the Dako CoverStainer (batch stainer) from Agilent Technologies; the Leica ST4020 Small Linear Stainer (batch stainer), Leica ST5020 Multistainer (batch stainer), and the Leica ST5010 Autostainer XL series (batch stainer) H&E stainers from Leica Biosystems Nussloch GmbH.

### V. Staining Evaluation

In an embodiment, a set of stained samples generated by the presently disclosed methods are used to determine the presence or absence of a fusion protein in a patient sample. In the typical case, a sample obtained from a patient and prepared for analysis as set forth above. One portion of the sample (for example, a first tissue section of a biopsy of tumor resection sample, or a first slide prepared from a cytological sample of tumor cells (such as cellular smears (such as cervical smears), fine needle aspirates, isolated circulating tumor cells and the like) are prepared and stained with the first biomarker-specific reagent. A second portion of the same sample (for example, a serial section of the first tissue section, or a second slide prepared from the same cytological sample) is stained with the second biomarker-specific reagent. The staining process is optimized to obtain intensity-matched staining with the first and second biomarker-specific reagents. The stained samples are then scored on the basis of intensity. The presence of a fusion protein is diagnosed by matched staining between the first and second biomarker-specific reagent. The absence of a fusion protein is diagnosed by observing mismatched staining between the first and second biomarker-specific reagent.

### V.A. Manual Scoring

In some embodiments, intensity scoring is performed manually by a trained reader. The trained reader evaluates the extent, intensity, and (for tissue sections) the localization of staining in the two samples. The two samples are considered to have matched staining when staining occurs to a similar extent, with similar relative intensity, and (for tissue sections) at similar locations. The two samples are considered to have mismatched staining when there is a difference in one or more of the extent, the localization, and the intensity of staining. Thus, for example, a trained reader reviews the stained samples and determines that, in the first portion, there is staining over 75% of the tumor area with an intensity level of 2+, and that in the second section, there is staining over 75% of the tumor area with an intensity level of 2+. This would be considered “matched staining,” and the sample would be determined to express a wild-type counterpart, but to not express the fusion protein. As another example, a trained reader reviews the stained samples and determines that, in the first portion, there is staining over 75% of the tumor area with an intensity level of 3+, and that in the second section, there is staining over 75% of the tumor area with an intensity level of 2+. This would be considered “mismatched staining,” and the sample would be determined to express a fusion protein. As another example, a trained reader reviews the stained samples and determines that, in the first portion, there is staining over 75% of the tumor area with an intensity level of 3+, and that in the second section, there is staining over 50% of the tumor area with an intensity level of 3+. This also would be considered “mismatched staining,” and the sample would be determined to express a fusion protein. As another example, a trained reader reviews the stained samples and determines that, in the first portion, there is staining over 75% of the tumor area with an intensity level of 3+, with staining occurring in both the tumor core and the invasive margin, and that in the second section, there is staining over 75% of the tumor area with an intensity level of 3+, but that all staining is confined to the tumor core, and no staining is observed in the invasive margin. This also would be considered “mismatched staining,” and the sample would be determined to express a fusion protein.

Examples of scoring methodologies include intensity score (typically on a 0, 1+, 2+, 3+ scale), percent intensity (i.e. percentage of relevant tissue compartment—such as tumor area, stroma, extracellular space, etc.—that stains above a specified intensity level), H-score (H-score=1(percentage of cells with 1+ staining)+2(percentage of relevant cells with 2+ staining)+3(percentage of relevant cells with 2+ staining)).

Scoring is typically performed only on cells that express the biomarker of interest in a cellular compartment in which the biomarker is expected to be expressed. Exemplary compartments in which specific biomarkers are expected to be expressed are set forth in Table 7:

TABLE 7

| | | Exemplary “positive” and |
|---|---|---|
| Wild-type | | “negative” scoring |
| protein | Expected expression pattern | methodologies |
| ROS1 | Equivalent staining of N-ROS | Decrease staining (intensity |
| | and C-ROS in the cytoplasmic | and/or percent tumor staining) |
| | compartment for wild type | in the N-ROS when compared |
| | expression | to C-ROS as positive for a |
| | | fusion. Negative for fusion |
| | | would be no staining or |
| | | equivalent staining of N-ROS |
| | | and C-ROS |
| RET | Equivalent staining of N-RET | Un-equivalent (intensity and/or |
| | and C-RET in the cytoplasmic | percent tumor staining) in the |
| | and/or membranous | N-RET when compared to C- |
| | compartment for wild type | RET as positive for a fusion. |
| | expression | Negative for fusion would be |
| | | no staining or equivalent |
| | | staining of N-RET and C-RET |
| ALK | Equivalent staining of N-ALK | Un-equivalent (intensity and/or |
| | and C-ALK in the membranous | percent tumor staining) in the |
| | compartment for wild type | N-ALK when compared to C- |
| | expression | ALK as positive for a fusion. |
| | | Negative for fusion would be |
| | | no staining or equivalent |
| | | staining of N-ALK and C-ALK |
| TrkA | Equivalent staining of N-TrkA | Un-equivalent (intensity and/or |
| | and C-TrkA in the cytoplasmic | percent tumor staining) in the |
| | compartment for wild type | N-TrkA when compared to C- |
| | expression | TrkA as positive for a fusion. |
| | | Negative for fusion would be |
| | | no staining or equivalent |
| | | staining of N-TrkA and C- |
| | | TrkA |
| TrkB | Equivalent staining of N-TrkB | Un-equivalent (intensity and/or |
| | and C-TrkB in the cytoplasmic | percent tumor staining) in the |
| | compartment for wild type | N-TrkB when compared to C- |
| | expression | TrkB as positive for a fusion. |
| | | Negative for fusion would be |
| | | no staining or equivalent |
| | | staining of N-TrkB and C- |
| | | TrkB |
| TrkC | Equivalent staining of N-TrkC | Un-equivalent (intensity and/or |
| | and C-TrkC in the cytoplasmic | percent tumor staining) in the |
| | compartment for wild type | N-TrkC when compared to C- |
| | expression | TrkC as positive for a fusion. |
| | | Negative for fusion would be |
| | | no staining or equivalent |
| | | staining of N-TrkC and C- |
| | | TrkC |
| RAF1 | Equivalent staining of N-RAF1 | Un-equivalent (intensity and/or |
| | and C-RAF1 in the nuclear | percent tumor staining) in the |
| | compartment for wild type | N-RAF1 when compared to C- |
| | expression | RAF1 as positive for a fusion. |
| | | Negative for fusion would be |
| | | no staining or equivalent |
| | | staining of N-RAF1 and C- |
| | | RAF1 |
| BRAF | Equivalent staining of N-BRAF | Un-equivalent (intensity and/or |
| | and C-BRAF in the cytoplasmic | percent tumor staining) in the |
| | compartment for wild type | N-BRAF when compared to C- |
| | expression | BRAF as positive for a fusion. |
| | | Negative for fusion would be |
| | | no staining or equivalent |
| | | staining of N-BRAF and C- |
| | | BRAF |
| PRKCA | Equivalent staining of N- | Un-equivalent (intensity and/or |
| | PRKCA and C-PRKCA in the | percent tumor staining) in the |
| | cytoplasmic compartment for | N-PRKCA when compared to |
| | wild type expression | C-PRKCA as positive for a |
| | | fusion. Negative for fusion |
| | | would be no staining or |
| | | equivalent staining of N- |
| | | PRKCA and C-PRKCA |
| PRKCB | Equivalent staining of N- | Un-equivalent (intensity and/or |
| | PRKCB and C-PRKCB in the | percent tumor staining) in the |
| | cytoplasmic and/or nuclear | N-PRKCB when compared to |
| | compartment for wild type | C-PRKCB as positive for a |
| | expression | fusion. Negative for fusion |
| | | would be no staining or |
| | | equivalent staining of N- |
| | | PRKCB and C-PRKCB |
| PKN1 | Equivalent staining of N- | Un-equivalent (intensity and/or |
| | PKN1and C-PKN1 in the | percent tumor staining) in the |
| | cytoplasmic compartment for | N-PKN1 when compared to C- |
| | wild type expression | PKN1 as positive for a fusion. |
| | | Negative for fusion would be |
| | | no staining or equivalent |
| | | staining of N-PKN1 and C- |
| | | PKN1 |

In an embodiment, the presence or absence of a fusion protein involving a wild-type counterpart of Table 7 is detected by detecting matched staining in one or more of the corresponding cellular compartments of Table 7.

### V.B. Automated or Semi-Automated Scoring

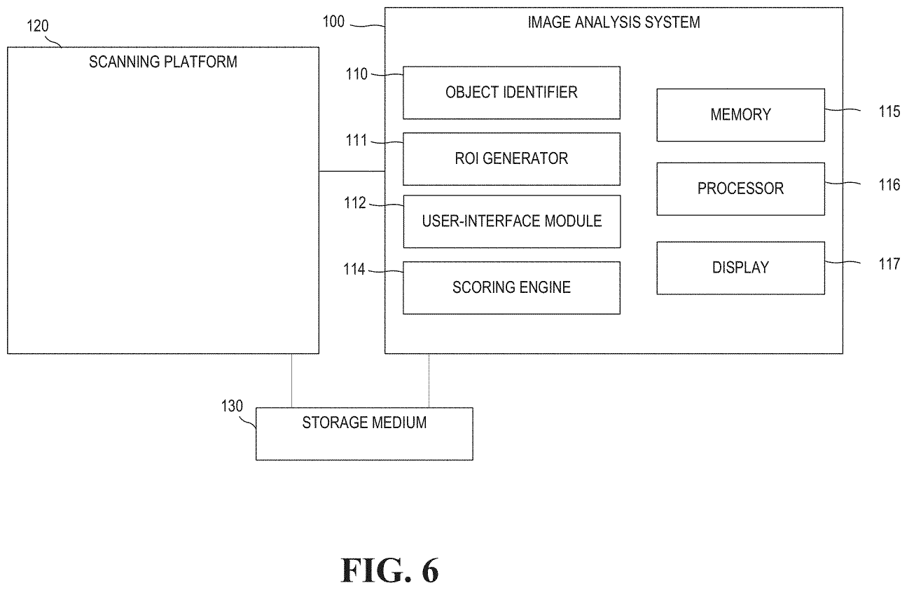

In some embodiments, scoring may be performed by a digital pathology system. These systems typically include an image acquisition component and an image analysis component. An exemplary digital pathology system is illustrated at FIG. 6.

Scanning platform 120 typically includes at least a scanning platform 120 such as a slide scanner that can scan the stained slides at 20×, 40×, or other magnifications to produce high resolution whole-slide digital images. At a basic level, the typical slide scanner includes at least: (1) a microscope with lens objectives, (2) a light source (such as halogen, light emitting diode, white light, and/or multispectral light sources, depending on the dye), (3) robotics to move glass slides around (or to move the optics around the slide), (4) one or more digital cameras for image capture, (5) a computer and associated software to control the robotics and to manipulate, manage, and view digital slides. Digital data at a number of different X-Y locations (and in some cases, at multiple Z planes) on the slide are captured by the camera's charge-coupled device (CCD), and the images are joined together to form a composite image of the entire scanned surface. Common methods to accomplish this include:
(1) Tile based scanning, in which the slide stage or the optics are moved in very small increments to capture square image frames, which overlap adjacent squares to a slight degree. The captured squares are then automatically matched to one another to build the composite image; and
(2) Line-based scanning, in which the slide stage moves in a single axis during acquisition to capture a number of composite image “strips.” The image strips can then be matched with one another to form the larger composite image.
   A detailed overview of various scanners (both fluorescent and brightfield) can be found at Farahani et al., Whole slide imaging in pathology: advantages, limitations, and emerging perspectives, Pathology and Laboratory Medicine Intl, Vol. 7, p. 23-33 (June 2015), the content of which is incorporated by reference in its entirety. Examples of commercially available slide scanners include: 3DHistech PANNORAMIC SCAN II; DigiPath PATHSCOPE; Hamamatsu NANOZOOMER RS, HT, and XR; Huron TISSUESCOPE 4000, 4000XT, and HS; Leica SCANSCOPE AT, AT2, CS, FL, and SCN400; Mikroscan D2; Olympus VS120-SL; Omnyx VL4, and VL120; PerkinElmer LAMINA; Philips ULTRA-FAST SCANNER; Sakura Finetek VISIONTEK; Unic PRECICE 500, and PRECICE 600×; VENTANA DP200, ISCAN COREO and ISCAN HT; and Zeiss AXIO SCAN.Z1. Other exemplary systems and features can be found in, for example, WO2011-049608 or in U.S. Patent Application No. 61/533,114, filed on Sep. 9, 2011, entitled IMAGING SYSTEMS, CASSETTES, AND METHODS OF USING THE SAME the content of which is incorporated by reference in its entirety.

Images generated by scanning platform 120 may be transferred to image analysis system 100 or to a storage medium 130 (such as a server, database, or non-transitory computer readable media) which is accessible and/or readable by image analysis system 100. In some embodiments, the images may be transferred automatically to image analysis system 100 via one or more local-area networks and/or wide-area networks. In some embodiments, image analysis system 100 may be integrated with or included in scanning platform 120 and/or other modules of scanning platform 120, in which case the image may be transferred to image analysis system, e.g., through a memory accessible by both scanning platform 120 and system 120. In some embodiments, scanning platform 120 may not be communicatively coupled to image analysis system 100, in which case the images may be stored on a non-volatile form of the storage medium 130 of any type (e.g., a flash drive) and downloaded from the medium to image analysis system 100 or to a server or database communicatively coupled thereto. In any of the above examples, image analysis system 100 may obtain an image of a biological sample, where the sample may have been affixed to a slide and stained by an advanced staining platform (not illustrated), and where the slide may have been scanned by a slide scanner 120 or another type of scanning platform. It is appreciated, however, that in other embodiments, below-described techniques may also be applied to images of biological samples acquired and/or stained through other means.

The digital pathology system includes an image analysis system 100. Image analysis system 100 may include one or more computing devices such as desktop computers, laptop computers, tablets, smartphones, servers, application-specific computing devices, or any other type(s) of electronic device(s) capable of performing the techniques and operations described herein. In some embodiments, image analysis system 100 may be implemented as a single device. In other embodiments, image analysis system 100 may be implemented as a combination of two or more devices together achieving the various functionalities discussed herein. For example, image analysis system 100 may include one or more server computers and a one or more client computers communicatively coupled to each other via one or more local-area networks and/or wide-area networks such as the Internet.

As illustrated in FIG. 6, image analysis system 100 may include a memory 116, a processor 117, and a display 118. Memory 116 may include any combination of any type of volatile or non-volatile memories, such as random-access memories (RAMs), read-only memories such as an Electrically-Erasable Programmable Read-Only Memory (EEPROM), flash memories, hard drives, solid state drives, optical discs, and the like. For brevity purposes memory 116 is depicted in FIG. 6 as a single device, but it is appreciated that memory 116 can also be distributed across two or more devices.

Processor 117 may include one or more processors of any type, such as central processing units (CPUs), graphics processing units (GPUs), special-purpose signal or image processors, field-programmable gate arrays (FPGAs), tensor processing units (TPUs), and so forth. For brevity purposes processor 117 is depicted in FIG. 6 as a single device, but it is appreciated that processor 117 can also be distributed across any number of devices.

Display 118 may be implemented using any suitable technology, such as LCD, LED, OLED, TFT, Plasma, etc. In some implementations, display 118 may be a touch-sensitive display (a touchscreen).

As illustrated in FIG. 6, image analysis system 100 may also include an object identifier 110, a region of interest (ROI) generator 111, a user-interface module 112, and a scoring engine 114. While these modules are depicted in FIG. 6 as standalone modules, it will be evident to persons having ordinary skill in the art that each module may instead be implemented as a number of sub-modules, and that in some embodiments any two or more modules can be combined into a single module. Furthermore, in some embodiments, system 100 may include additional engines and modules (e.g., input devices, networking and communication modules, etc.) not depicted in FIG. 6 for brevity. Furthermore, in some embodiments, some of the blocks depicted in FIG. 6 may be disabled or omitted. As will be discussed in more detail below, the functionality of some or all modules of system 100 can be implemented in hardware, software, firmware, or as any combination thereof. Exemplary commercially-available software packages useful in implementing modules as disclosed herein include VENTANA VIRTUOSO; Definiens TISSUE STUDIO, DEVELOPER XD, and IMAGE MINER; and Visopharm BIOTOPIX, ONCOTOPIX, and STEREOTOPIX software packages.

After acquiring the image, image analysis system 100 may pass the image to an object identifier 110, which functions to identify and mark relevant objects and other features within the image that will later be used for scoring. Object identifier 110 may extract from (or generate for) each image a plurality of image features characterizing the various objects in the image as a well as pixels representing expression of the biomarker(s). The extracted image features may include, for example, texture features such as Haralick features, bag-of-words features and the like. The values of the plurality of image features may be combined into a high-dimensional vector, hereinafter referred to as the “feature vector” characterizing the expression of the biomarker. For example, if M features are extracted for each object and/or pixel, each object and/or pixel can be characterized by an M-dimensional feature vector. The output of object identifier 110 is effectively a map of the image annotating the position of objects and pixels of interest and associating those objects and pixels with a feature vector describing the object or pixels.

For biomarkers that are scored on the basis of the biomarker's association with a particular type of object (such as membranes, nuclei, cells, etc.), the features extracted by object identifier 110 may include features or feature vectors sufficient to categorize the objects in the sample as biomarker-positive objects of interest or biomarker-negative markers of interest and/or by level or intensity of biomarker staining of the object. In cases where the biomarker may be weighted differently depending on the object type that is expressing it (for example, scored on the basis of tumor cell expression versus stromal expression), the features extracted by object identifier 110 may include features relevant to determining the type of objects associated with biomarker-positive pixels. Thus, the objects may then be categorized at least on the basis of biomarker expression (for example, biomarker-positive or biomarker-negative cells) and, if relevant, a subtype of the object (e.g. tumor cell, immune cell, etc.). In cases where extent of biomarker-expression is scored regardless of association with objects, the features extracted by object identifier 110 may include for example location and/or intensity of biomarker-positive pixels. The precise features extracted from the image will depend on the type of classification function being applied, and would be well-known to a person of ordinary skill in the art.

### VI. Clinical Application

In an embodiment, the assay as described herein is used to characterize a tumor sample from a patient. For example, a biopsy section or a resection sample is obtained, fixed, embedded in paraffin, and sectioned. Serial sections are stained with the first and second biomarker-specific reagents and staining intensity is scored. A tumor having a score indicative of the presence of a fusion protein is characterized as “fusion positive,” while a tumor having a score that is not indicative of the presence of a fusion protein is characterized as “fusion negative.” In another embodiment, a fine needle aspirate (FNA) of a solid tumor is obtained and at least two slides created from the sample. The slides are separately stained with the first and second biomarker-specific reagents and a number of tumor cells staining positively with each biomarker-specific reagent is scored as a function of the total number of tumor cells. An FNA sample having a score indicative of the presence of a fusion protein is characterized as “fusion positive,” while an FNA sample having a score that is not indicative of the presence of a fusion protein is characterized as “fusion negative.”

In some embodiments, the assay is used as a screening test to identify patients eligible for a nucleic acid-based assay to confirm the presence of the fusion protein. For example, samples may be screened for the presence or absence of a fusion protein using the assay, and only those samples that are characterized as fusion positive are subjected to a sequencing-based or PCR-based assay to confirm the presence and/or identity of the fusion that is was detected in the assay. In other embodiments, the assay is a reflex test to confirm the presence and expression of a fusion protein identified by a nucleic acid-based assay. For example, samples may be screened for the presence or absence of a fusion protein using a sequencing-based or PCR-based assay, and only those samples that are characterized as fusion positive by the sequencing-based or PCR-based assay are screened by the assay described herein to confirm the presence and/or expression of the fusion detected by the nucleic acid assay. In other embodiments, characterization of the presence or absence of a fusion protein is made solely on the basis of the assay.

In some embodiments, the assay is used to select a therapy for the patient. For example, a patient having a tumor or sample characterized as “fusion positive” receives a targeted therapy directed against the wild-type counterpart, optionally in combination with a standard treatment course for the tumor. Exemplary targeted therapies include those recited in Table 8:

TABLE 8

| Wild-type protein | | Exemplary active ingredients or |
|---|---|---|
| (References) | Drug type | compound class |
| ROS1 | Small molecule | crizotinib, lorlatinib, ceritinib, entrectinib |
| (Roskoski 1) | tyrosine kinase | cabozantinib |
| | inhibitor | |
| RET | Small molecule | cabozantinib, vandetanib, lenvatinib, |
| (Roskoski 2) | tyrosine kinase | alectinib, sunitinib, sorafenib |
| | inhibitor | Ponatinib |
| ALK | Small molecule | Crizotinib, ceritinib, alectinib, brigatinib, |
| (Roskoski 3) | tyrosine kinase | entrectinib, lorlatinib |
| | inhibitor | |
| TrkA/B/C | Small molecule | aliratinib; belizatinib; cabozantinib; |
| (Bailey) | tyrosine kinase | dovitinib; DS-6051b; entrectinib; |
| | inhibitor | F17752; LOXO-101 (larotrectinib); |
| | | milciclib; PLX7486; sitrivatinib |
| RAF1 | Small molecule | sorafenib, AZ628, Raf265, AAL881, |
| | serine/threonine kinase | LBT613 |
| | inhibitor | |
| BRAF | Small molecule | vemurafenib, dabrafenib, sorafenib, |
| (Khazak) | serine/threonine kinase | PLX4032, AAL881, LBT613 |
| | inhibitor | |
| PRKCA/ | Small molecule | UCN-01, chelerythrine, UCN-01, |
| PRKCB/ | serine/threonine kinase | Gö6976, Bisindolylmaleimide I (BIM 1) |
| PKN1 | inhibitor | and PKC412 (midostaurin); AEB071 |
| (Storz) | | |

A patient having a tumor or sample characterized as fusion negative receives a standard therapy, without inclusion of a targeted therapy for the wild-type counterpart.

### VII. Kits and Staining Assemblies

In an embodiment, a kit is provided for performing the staining methods as described herein. In an embodiment, the kit comprises a first biomarker-specific reagent and a second biomarker-specific reagent. In an embodiment, the first and second biomarker-specific reagents are specific for a wild-type protein set forth in Table 1. In an embodiment, the first and second biomarker-specific reagents are specific for a wild-type protein set forth in Table 2, wherein the first biomarker-specific reagent is specific for the consensus retained portion of the wild-type protein and the second biomarker-specific reagent is specific for the consensus lost portion of the wild-type protein. In an embodiment, the first and second biomarker-specific reagents are specific for a wild-type protein set forth in Table 3, wherein the first biomarker-specific reagent is specific for the consensus retained portion of the wild-type protein and the second biomarker-specific reagent is specific for the consensus lost portion of the wild-type protein. In an embodiment, the first and second biomarker-specific reagents are specific for a wild-type protein set forth in Table 4, wherein the first biomarker-specific reagent is specific for an N-terminal portion of the wild-type protein and the second biomarker-specific reagent is specific for a C-terminal portion of the wild-type protein, and wherein neither the first nor the second biomarker-specific reagent is specific for an excluded portion of the wild-type protein. In an embodiment, the first and second biomarker-specific reagents are specific for a wild-type protein set forth in Table 5, wherein the first biomarker-specific reagent is an N-terminus directed antibody and the second biomarker-specific reagent is a C-terminus directed antibody. In an embodiment, the first and second biomarker-specific reagents are provided in a ready-to-use format at a titer that achieves intensity-matched staining with a specific set of detection reagents. In another embodiment, the first and second biomarker-specific reagents are provided as a concentrate or solid form (such as a lyophilate, crystallized, or powdered composition, or other ready-to-dissolve solid). In an embodiment, kits comprising the concentrate or solid form of the first and second biomarker-specific reagents further comprise instructions for obtaining a final titer of the first and second biomarker-specific reagents that achieves intensity-matched staining with a specific set of staining reagents. In an embodiment, kits comprising the concentrate or solid form of the first and second biomarker-specific reagents further comprise a diluent for each of the first and second biomarker-specific reagents. In another embodiment, the first and second biomarker-specific reagents are provided as the concentrate or solid form, and the kits further comprise a diluent for each of the first and second biomarker-specific reagents. In some embodiments, the diluents are provided in a pre-measured volume, wherein the pre-measured volume is a volume that achieves a final titer of the first and second biomarker-specific reagents that achieves intensity-matched staining with a specific set of staining reagents. In some embodiments, the diluents are provided in excess, wherein the kit further comprises instructions for using the diluents to obtain a final titer of the first and second biomarker-specific reagents that achieves intensity-matched staining with a specific set of staining reagents. In some embodiments, kits of any of the foregoing embodiments may further comprise instructions for achieving intensity-matched staining with the first and second biomarker-specific reagents. Additionally, in any of the foregoing embodiments, the kits may further comprise the specific set of detection reagents. In an embodiment, the specific set of detection reagents is a set of detection reagents for performing one of the staining methodologies of Table 6 with each of the biomarker-specific reagents. In an embodiment, the biomarker-specific reagents and specific detection reagents (if included) of the kit are antibodies. In an embodiment in which the kit comprises a set of staining reagents, the staining reagents are suitable for brightfield microscopy.

In an embodiment, an assembly for performing intensity-matched staining is provided, the assembly generally comprising an automated advanced staining platform programmed to perform an intensity matched staining process with a first biomarker-specific reagent and a second biomarker-specific reagent as set forth herein. In an embodiment, the assembly comprises: (a) a first biomarker-specific reagent diluted to a final titer in a first diluent; (b) a second biomarker-specific reagent diluted to a final titer in a second diluent, which may be the same as or different from the first diluent; (c) a set of detection reagents for the first biomarker-specific reagent; (d) a set of detection reagents for the second biomarker-specific reagent; (e) a set of samples obtained from the same tumor; and (f) an automated advanced staining platform programmed to apply (a) and (c) to a first portion of the set of samples and to apply (d) to (e) to a second portion of the set of samples using a protocol that obtains intensity-matched staining. In an embodiment, the first and second biomarker-specific reagents are specific for a wild-type protein set forth in Table 1. In an embodiment, the first and second biomarker-specific reagents are specific for a wild-type protein set forth in Table 2, wherein the first biomarker-specific reagent is specific for the consensus retained portion of the wild-type protein and the second biomarker-specific reagent is specific for the consensus lost portion of the wild-type protein. In an embodiment, the first and second biomarker-specific reagents are specific for a wild-type protein set forth in Table 3, wherein the first biomarker-specific reagent is specific for the consensus retained portion of the wild-type protein and the second biomarker-specific reagent is specific for the consensus lost portion of the wild-type protein. In an embodiment, the first and second biomarker-specific reagents are specific for a wild-type protein set forth in Table 4, wherein the first biomarker-specific reagent is specific for an N-terminal portion of the wild-type protein and the second biomarker-specific reagent is specific for a C-terminal portion of the wild-type protein, and wherein neither the first nor the second biomarker-specific reagent is specific for an excluded portion of the wild-type protein. In an embodiment, the first and second biomarker-specific reagents are specific for a wild-type protein set forth in Table 5, wherein the first biomarker-specific reagent is an N-terminus directed antibody and the second biomarker-specific reagent is a C-terminus directed antibody. In an embodiment, the set of samples of (e) are serial sections of a formalin-fixed paraffin-embedded tissue section, and the detection reagents of (c) and (d) are reagents for deposition of chromogenic or fluorescent dyes. In some embodiments, the set of samples of (e) are serial sections of a formalin-fixed paraffin-embedded tissue section, the first and second biomarker-specific reagents are primary antibodies, and the detection reagents of (c) and (d) comprise secondary antibodies specific for the primary antibodies and additional reagents for enzymatic deposition of chromogenic or fluorescent dyes. In some embodiments, the set of samples of (e) are serial sections of a formalin-fixed paraffin-embedded tissue section, the first and second biomarker-specific reagents are primary antibodies, and the detection reagents of (c) and (d) are detection reagents for performing one of the detection schemes according to Table 6. In some embodiments, the set of samples of (e) are serial sections of a formalin-fixed paraffin-embedded tissue section, the first and second biomarker-specific reagents are primary antibodies, and the detection reagents of (c) and (d) are detection reagents for performing one of the detection schemes according to Table 6 using DAB as the dye.

In another embodiment, an assembly for detecting the presence or absence of a fusion protein in a sample is provided, the assembly generally comprising: (a) a set of histologically or cytologically stained slides, the set of histologically or cytologically-stained slides comprising a first slide stained with a first biomarker-specific reagent as set forth herein and a second slide stained with a second biomarker-specific reagent as set forth herein, the first and second slides being stained by a process that achieves intensity-matched staining; and (b) an imaging system. In an embodiment, the imaging system comprises a microscope suitable for manual scoring of the stained slides. In another embodiment, the imaging system comprises an automated slide scanner suitable for generating a digital image of each slide. In another embodiment, the assembly further comprises (c) an image analysis system communicatively coupled to the automated slide scanner, the image analysis system programmed to generate an intensity score for each of the digital images of the first and second slides. In another embodiment, the image analysis system of (c) is programmed to calculate a score selected from the group consisting of: relative intensity score (typically on a 0, 1+, 2+, 3+ scale), percent intensity (i.e. percentage of relevant tissue compartment—such as tumor area, stroma, extracellular space, etc.—that stains above a specified intensity level), H-score (H-score=1(percentage of relevant cells with 1+ staining)+2(percentage of relevant cells with 2+ staining)+3(percentage of relevant cells with 2+ staining)). In another embodiment, the image analysis system is programmed to generate a score according to one or more of the fusion partners and methodologies set forth in Table 7.

In another embodiment, an assembly for scoring a stained slide is provided, the assembly generally comprising: (a) a set of digital images stored on a non-transitory computer readable memory, the set of digital images comprising one or more images of each of a set of histologically or cytologically stained slides, the set of histologically or cytologically-stained slides comprising a first slide stained with a first biomarker-specific reagent as set forth herein and a second slide stained with a second biomarker-specific reagent as set forth herein, the first and second slides being stained by a process that achieves intensity-matched staining; and (b) an imaging analysis system programmed to generate an intensity-based score for each of the digital images of the first and second slides. In another embodiment, the image analysis system of (c) is programmed to calculate a score selected from the group consisting of: relative intensity score (typically on a 0, 1+, 2+, 3+ scale), percent intensity (i.e. percentage of relevant tissue compartment—such as tumor area, stroma, extracellular space, etc.—that stains above a specified intensity level), H-score (H-score=1 (percentage of relevant cells with 1+ staining)+2(percentage of relevant cells with 2+ staining)+3(percentage of relevant cells with 2+ staining)). In another embodiment, the image analysis system is programmed to generate a score according to one or more of the methodologies set forth in Table 7.

### VIII. Examples

The following examples use N- and C-terminal antibodies against human ROS1 as a model system to test whether ROS1 fusion proteins could be detected in an immunohistochemical format on the basis of relative intensity.

### VIII.A. Antibodies, Antibody Titer, and Staining Method

Rabbit monoclonal antibodies specific for N-Ros1 and C-Ros1 were raised against immunogens comprising amino acids 395-418 (N-Ros1) and 2336-2347 (c-Ros1) of SEQ ID NO: 1. The antibodies were intensity matched using two multi-tissue blocks. One block included three pieces of tissue, two known to be positive for fusion protein via fluorescent in situ hybridization (FISH) and one with unknown FISH status. The other block had 4 pieces of tissue with unknown FISH status and reactive type-two pneumocytes. Sections were obtained from the blocks and mounted on positively charged glass slides. The titer of the c-Ros1 antibody was optimized for maximal staining intensity in samples containing fusion or wild-type without off-target staining, which in this case was a 1:10,000 dilution in Tris-HCL Dilution Buffer with Brij-35. Serial dilutions of the N-Ros1 antibody were selected, and serial sections were matched and stained with n-Ros and c-Ros antibodies at the titers listed in Table 9 in Tris-HCL Dilution Buffer with Brij-35:

TABLE 9

| | Section 1 | Section 2 |
|---|---|---|
| Pair #1 | C-Ros1 at 1:10,000 | n-Ros1 at 1:50 |
| Pair #2 | C-Ros1 at 1:10,000 | n-Ros1 at 1:100 |
| Pair #3 | C-Ros1 at 1:10,000 | n-Ros1 at 1:500 |
| Pair #4 | C-Ros1 at 1:10,000 | n-Ros1 at 1:1000 |
| Pair #5 | C-Ros1 at 1:10,000 | n-Ros1 at 1:2000 |
| Pair #6 | C-Ros1 at 1:10,000 | n-Ros1 at 1:5000 |
| Pair #7 | C-Ros1 at 1:10,000 | n-Ros1 at 1:10,000 |
| Pair #8 | C-Ros1 at 1:10,000 | n-Ros1 at 1:20,000 |

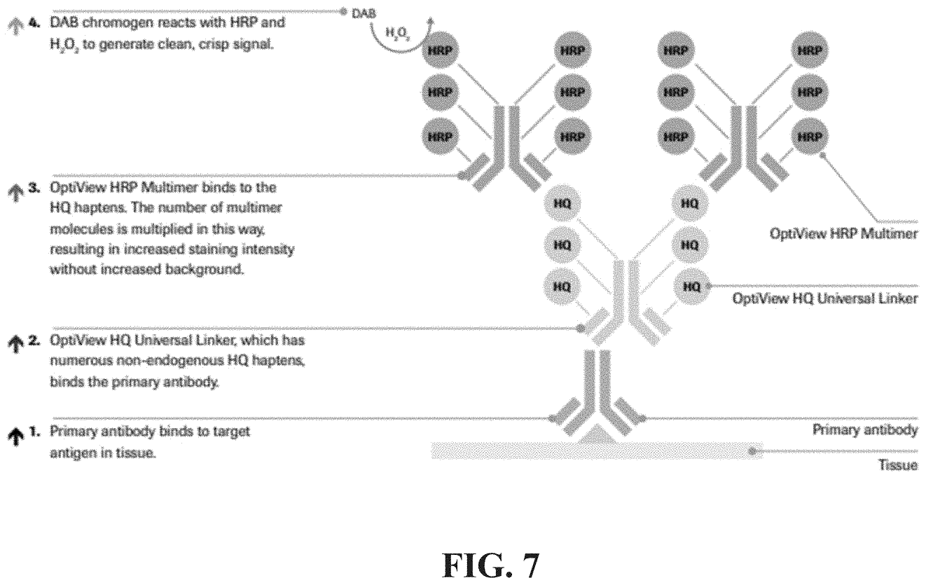

Staining was performed on a BenchMark ULTRA automated slide stainer (Ventana Medical Systems, Inc.) using OptiView DAB IHC detection kit (Ventana Medical Systems, Inc.). A schematic of the OptiView DAB IHC detection system is illustrated at FIG. 7. The staining protocol is listed below at Table 10:

TABLE 10

| Parameter | BenchMark ULTRA |
|---|---|
| Deparaffinization | Selected |
| Cell Conditioning (CC1) | 64 minutes |
| Pre Primary Peroxidase Inhibitor | Selected |
| Primary Antibody or Negative | 16 minutes @ 36° C. |
| Control Ig | |
| OptiView HQ Universal Linker | 8 minutes |
| OptiView HRP Multimer | 8 minutes |
| Counterstain: Hematoxylin II | 4 minutes |
| Post Counterstain: Bluing Reagent | 4 minutes |

CC1 is a tris based buffer with a slightly basic pH, which, at elevated temperatures is capable of hydrolyzing the covalent bonds formed by formalin in tissue.

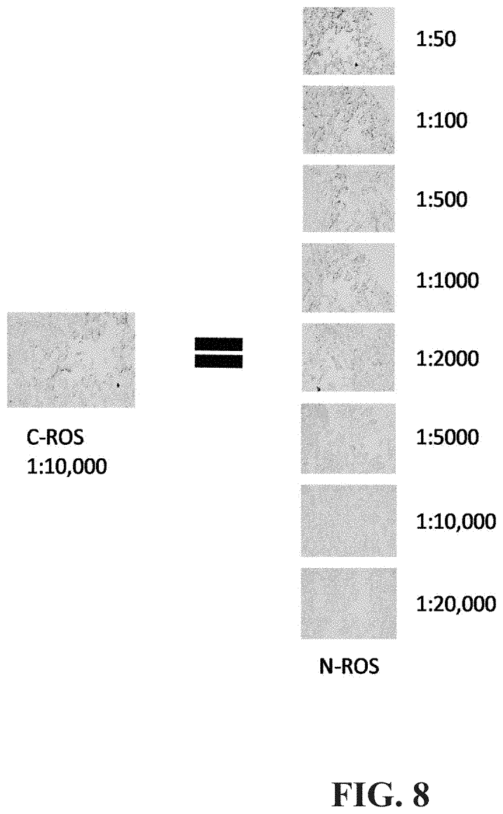

Staining in the samples of each pair was reviewed by a trained pathologist for: (a) closeness of staining intensity in samples containing wild-type Ros1; and (b) observable difference in staining of samples harboring Ros1 fusion-positive samples. Images of the stained slides can be seen at FIG. 8. Pair #5 was deemed to have the closest match between the two antibodies staining in wild-type samples, with acceptable background and off-target staining levels in fusion-positive and Ros1-negative samples. A 1:2000 titer of the n-Ros1 antibody and a 1:10,000 titer of the c-Ros antibody was selected for subsequent experiments.

### VIII.B. Test Samples

9 formalin-fixed, paraffin-embedded non-small cell lung carcinoma (NSCLC) tumor resections were selected to test the ability of the present methods to distinguish fusion-positive from fusion-negative cases. Cases were tested for the presence of ROS1 genetic rearrangements by FISH. Some samples (including all samples testing negative by FISH) were also tested by RT-PCR. Samples testing positive in at least one of the FISH and RT-PCR tests were considered “positive,” while samples negative by both FISH and RT-PCR were considered negative. One sample, with discrepant FISH status and RT-PCR status, was also tested by DNA sequencing. Samples and fusion status are set forth in Table 11:

TABLE 11

| Case ID | FISH Status | RT-PCR status | Sequencing |
|---|---|---|---|
| 1 | Negative | Negative | N/A |
| 2 | Negative | Positive | Positive |
| 3 | Negative | Negative | N/A |
| 4 | Positive | N/A | N/A |
| 5 | Positive | Positive | N/A |
| 6 | Positive | N/A | N/A |
| 7 | Positive | N/A | N/A |
| 8 | Positive | N/A | N/A |
| 9 | Positive | Positive | N/A |

As can be seen, 2 samples (Case ID 1 and Case ID 3) are negative and 7 cases (Case ID Nos. 2 and 4-9) are positive.

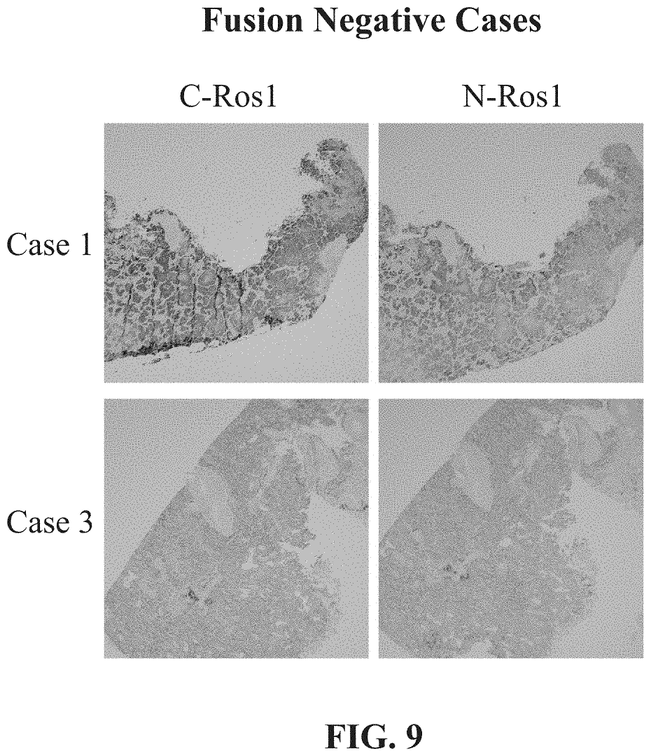

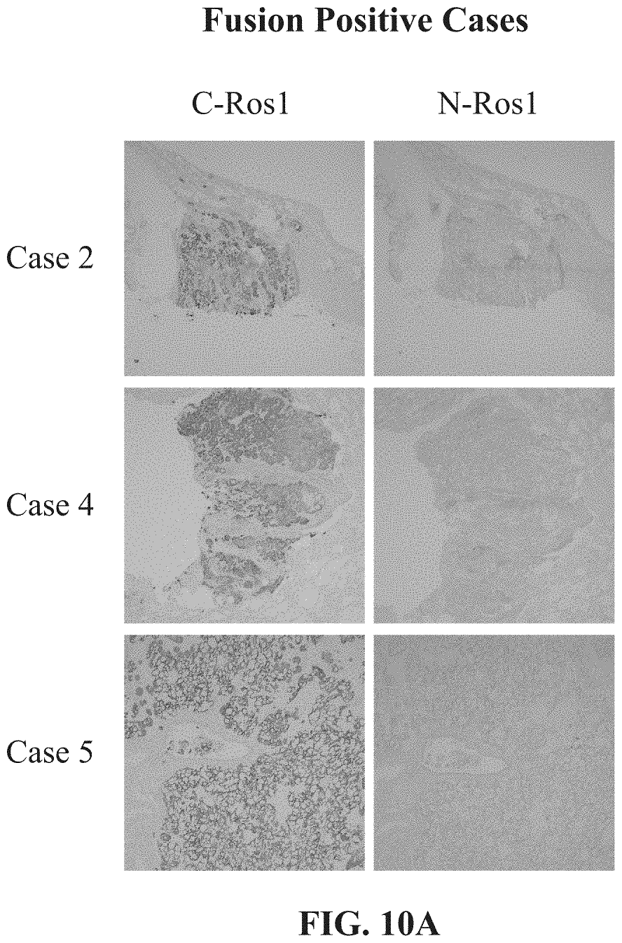

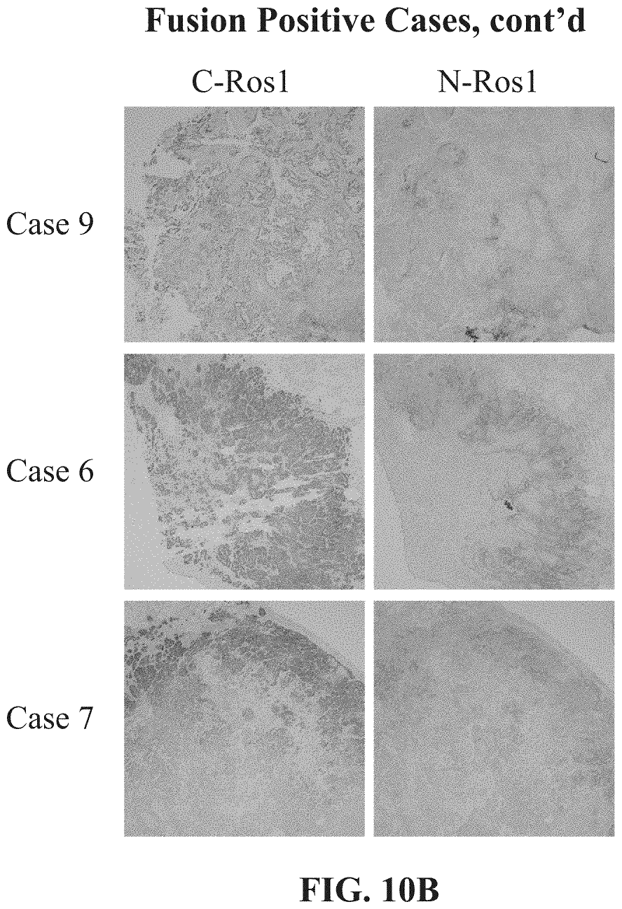

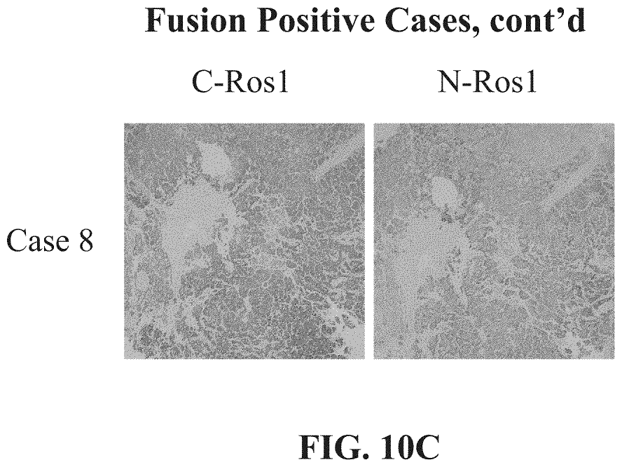

Consecutive sections of each case were stained as described in Section VIII.A, using either (a) a 1:10,000 dilution of the c-Ros1 antibody, or (b) a 1:2,000 dilution of the n-Ros1 antibody. Stained samples were scored by a pathologist on a 0+, 1+, 2+, 3+ scale. Images for each of the cases can be found at FIGS. 9 and 10A-10C. Scores for each case are shown below in Table 12:

TABLE 12

| Case ID | n-Ros1 Score | c-Ros1 score | FISH/RT-PCR status |
|---|---|---|---|
| 1 | 100%; 2.75 | 100%; 3.00 | Negative/Negative |
| | intensity | intensity | |
| 2 | 100%; 3.00 | 0%; 0 intensity | Negative/Positive |
| | intensity | | |
| 3 | 100%; 2.00 | 100%; 2.00 | Negative/Negative |
| | intensity | intensity | |
| 4 | 100%; 3.00 | 0%; 0 intensity | Positive/N/A |
| | intensity | | |
| 5 | 100%; 3.00 | 100%; 0.25 | Positive/Positive |
| | intensity | intensity | |
| 6 | 100% 3.00 | 40%; 2.00 | Positive/N/A |
| | intensity | intensity | |
| 7 | 60%; 2.50 | 30%; 1.25 | Positive/N/A |
| | intensity | intensity | |
| 8 | 100%; 3.00 | 100%; 2.25 | Positive/N/A |
| | intensity | intensity | |
| 9 | 100%; 2.5 | 0%; 0 intensity | Positive/Positive |
| | intensity | | |

The results reported in Table 12 show concordance between IHC staining patterns and the presence of ROS1 rearrangement as detected by FISH and/or RT-PCR.

### IX. References

The following references are hereby incorporated by reference in their entirety:
Amatu et al., NTRK gene fusions as novel targets of cancer therapy across multiple tumour types, ESMO Open, Vol. 1, Issue 2, e000023 doi:10.1136/esmoopen-2015-000023 (2016).
Appiah-Kubi et al., Platelet-derived growth factor receptors (PDGFRs) fusion genes involvement in hematological malignancies, Critical Revs. Oncology/Hematology, Vol. 109, pp.
20-34 (2017).
Bailey et al., Tropomyosin receptor kinase inhibitors: an updated patent review for 2010-2016—Part II, Expert Opinion on Therapetuic Patents, Vol. 27, Issue 7, pp. 831-49 (March 2017).
Bubendorf, et al. Testing for ROS1 in non-small cell lung cancer: a review with recommendations, Virchows Arch, Vol. 469, pp. 489-503 (2016).
Cascone et al., Targeting RET Rearrangements in Non-Small Cell Lung Cancer, ASCO Daily News (online), https://am.asco.org/targeting-ret-rearrangements-non-small-cell-lung-cancer (dated May 25, 2017; last accessed Mar. 22, 2018).
Cooper & Paterson, Determination of the Specific Antibody Titer, Current Protocols in Molecular Biology, Vol. 50, Issue 1, pp. 11.17.1-11.17.13 (April 2000).
D'Amico et al., State of the art in antigen retrieval for immunohistochemistry, J Immunol Methods. Vol. 341(1-2), pp. 1-18 (Feb. 28, 2009).
Hechtman et al., Pan-Trk Immunohistochemistry Is an Efficient and Reliable Screen for the Detection of NTRK Fusions, Am. J. Surg. Path., Vol. 41, Issue 11, pp. 1547-51 (November 2017).
Khazak et al., Selective Raf Inhibition in Cancer Therapy, Expert Opinions in Therapeutic Targets, Vol. 11, Issue 12, pp. 1587-1609 (December 2007).
Konduri et al., EGFR Fusions as Novel Therapeutic Targets in Lung Cancer, Cancer Discovery, Vol. 6, Issue 6, pp. 601-11 (2016).
Latsheva & Babu, Discovering and understanding oncogenic gene fusions through data intensive computational approaches, Nucleic Acids Res., Vol. 44, Issue 10, pp. 4487-4503 (2016).
Le Rolle et al., Identification and characterization of RET fusions in advanced colorectal cancer, Oncotarget, Vol. 6, Issue 30, pp. 28929-37 (2015).
Melo et al., The ABL-BCR fusion gene is expressed in chronic myeloid leukemia, Blood, Vol. 81, Issue 1, pp. 158-65 (1993).
Palanisamy et al., Rearrangements of the RAF Kinase Pathway in Prostate Cancer, Gastric Cancer and Melanoma, Nature Medicine, Vol. 16, Issue 7, pp. 793-98 (2010).
Prichard, Overview of Automated Immunohistochemistry, Arch Pathol Lab Med., Vol. 138, pp. 1578-1582 (2014)
Roskoski, ROS1 protein-tyrosine kinase inhibitors in the treatment of ROS1 fusion protein-driven non-small cell lung cancers, Pharmacological Research, Volume 121, pp. 202-212 (July 2017) (“Roskoski 1”).
Roskoski, Role of RET protein-tyrosine kinase inhibitors in the treatment RET-driven thyroid and lung cancers, Pharmacological Research, Volume 128, pp. 1-17 (February 2018) (“Roskoski 2”).
Roskoski, Anaplastic lymphoma kinase (ALK) inhibitors in the treatment of ALK-driven lung cancers, Pharmacological Research, Volume 117, pp. 343-356 (March 2017) (“Roskoski 3”).
Ross et al., ALK Fusions in a Wide Variety of Tumor Types Respond to Anti-ALK Targeted Therapy, Oncologist, Vol. 22, Issue 12, pp. 1444-50 (e-published Oct. 27, 2017)
Ross et al., The distribution of BRAF gene fusions in solid tumors and response to targeted therapy, Int. J. Cancer, Vol. 138, Issue 4, pp. 881-90 (2016).
Shi et al., Antigen retrieval immunohistochemistry: review and future prospects in research and diagnosis over two decades, J Histochem Cytochem, Vol. 59, Issue 1, pp. 13-32 (January 2011).
Storz, Targeting Protein Kinase C subtypes in pancreatic cancer, Expert Rev Anticancer Ther. Vol. 15, Issue 4, pp. 433-438 (2015).
Stransky et al., The landscape of kinase fusions in cancer, Nature Communications, Vol. 5, Article No. 4846 (2014) doi:10.1038/ncomms5846.
Uguen & De Braekeleer, ROS1 fusions in cancer: a review, Future Oncol., Vol. 12, Issue 16, pp. 1911-28 (2016).
Vinod et al., A simple and effective heat induced antigen retrieval method, MethodsX, Vol. 3, pp. 315-19 (published online Apr. 8, 2016).
Warford et al., Antigen retrieval, blocking, detection and visualisation systems in immunohistochemistry: A review and practical evaluation of tyramide and rolling circle amplification systems, Methods, Vol. 70, Issue 1, pp. 28-33 (November 2014).
Wellcome Sanger Institute, COSMIC—the Catalogue of Somatic Mutations in Cancer (COSMIC database), available at http://cancer.sanger.ac.uk/cosmic/fusion (last accessed 23-Mar.-2018).
Yamashita et al., Mechanisms of Heat-induced Antigen Retrieval: Analyses In Vitro Employing SDS-PAGE and Immunohistochemistry, J. Histochemistry and Cytochemistry, Vol. 53, Issue 1, pp. 13-21 (2005).

1. A method of preparing a histological or cytological sample of a tumor for detection of a fusion protein, the method comprising:
affinity enzymatically staining a first portion of the sample with a first biomarker-specific reagent, wherein the first biomarker-specific reagent is specific for a retained portion of a wild-type protein, and
affinity enzymatically staining a second portion the sample with a second biomarker-specific reagent, wherein the second biomarker-specific reagent is specific for a lost portion of the wild-type protein,
wherein affinity enzymatically staining the first portion of the sample and affinity enzymatically staining the second portion the sample results in intensity-matched staining of the first portion of the sample and the second portion of the sample.

2. The method of claim 1, wherein affinity enzymatically staining the sample comprises an affinity histochemical assay.

3. The method of claim 2, wherein the affinity histochemical assay comprises:
(a) contacting the first section with a first antibody, wherein the first antibody is immunospecific for an epitope located in the retained portion of the wild-type protein;
(b) contacting the first section having the first antibody bound thereto with a first set of detection reagents under conditions sufficient to deposit a dye on the first section in proximity to the first antibody bound to the first section;
(c) contacting the second section with a second antibody, wherein the second antibody is immunospecific for an epitope located in the lost portion of the wild-type protein; and
(d) contacting the sample having the second antibody bound thereto with a second set of detection reagents under conditions sufficient to deposit the second dye on the sample in proximity to the second antibody bound to the sample.

4. The method of claim 3, wherein:
(b) the first set of detection reagents comprises:
(b1) a first secondary detection reagent capable of specifically binding to the first antibody,
(b2) a first enzyme bound to or adapted to be bound to the first secondary detection reagent, and
(b3) a first set of chromogenic or fluorescent reagents reactive with the first enzyme, wherein reaction of the first set of chromogenic or fluorescent reagents results in generation of the first dye and/or deposition of the first dye onto the sample; and
(e) the second set of detection reagents comprises:
(e1) a second secondary detection reagent capable of specifically binding to the second antibody;
(e2) a second enzyme bound to or adapted to be bound to the second secondary detection reagent; and
(e3) a second set of chromogenic or fluorescent reagents reactive with the second enzyme, wherein reaction of the second set of chromogenic or fluorescent reagents results in generation of the second dye and/or deposition of the second dye onto the sample.

5. The method of claim 4, wherein the first enzyme is conjugated to the first secondary reagent and/or the second enzyme is conjugated to the second secondary reagent.

6. The method of claim 5, wherein:
(b4) the first set of detection reagents further comprises a first signaling conjugate, the first signaling conjugate comprising:
(b4a) a latent reactive moiety reactive with the first enzyme to generate a reactive species capable of binding to the sample, and
(b4b) an element conjugated to the latent reactive moiety, the element selected from the group consisting of
the first dye,
the first enzyme, and
a member of a first specific binding pair; and/or
(e4) the second set of detection reagents further comprises a second signaling conjugate, the second signaling conjugate comprising:
(e4a) a latent reactive moiety reactive with the second enzyme to generate a reactive species capable of binding to the sample, and
(e4b) an element conjugated to the latent reactive moiety, the element selected from the group consisting of
the second dye,
the second enzyme, and
a first member of a second specific binding pair.

7. The method of claim 6, wherein:
(b4b) the element conjugated to the latent reactive moiety of the first signaling conjugate is the member of the first specific binding pair, and wherein the first set of detection reagents further comprises a second member of the first specific binding pair, wherein the second member of the first specific binding pair is selected from the group consisting of the first dye and the first enzyme; and/or
(e4b) the element conjugated to the latent reactive moiety of the second signaling conjugate is the member of the second specific binding pair, and wherein the second set of detection reagents further comprises a second member of the second specific binding pair, wherein the second member of the second specific binding pair is selected from the group consisting of the second dye and the second enzyme.

8. The method of claim 4, wherein:
(b2) the first enzyme is conjugated to a first tertiary detection reagent, wherein the first tertiary detection reagent is capable of specifically binding to the first secondary detection reagent; and/or
(e2) the second enzyme is conjugated to a second tertiary detection reagent, wherein the second tertiary detection reagent is capable of specifically binding to the second secondary detection reagent.

9. The method of claim 8, wherein:
(b4) the first set of detection reagents further comprises a first signaling conjugate, the first signaling conjugate comprising:
(b4a) a latent reactive moiety reactive with the first enzyme to generate a reactive species capable of binding to the sample, and
(b4b) an element conjugated to the latent reactive moiety, the element selected from the group consisting of
the first dye,
the first enzyme, and
a member of a first specific binding pair; and/or
(e4) the second set of detection reagents further comprises a second signaling conjugate, the second signaling conjugate comprising:
(e4a) a latent reactive moiety reactive with the second enzyme to generate a reactive species capable of binding to the sample, and
(e4b) an element conjugated to the latent reactive moiety, the element selected from the group consisting of
the second dye,
the second enzyme, and
a first member of a second specific binding pair.

10. The method of claim 9, wherein:
(b4b) the element conjugated to the latent reactive moiety of the first signaling conjugate is the member of the first specific binding pair, and wherein the first set of detection reagents further comprises a second member of the first specific binding pair, wherein the second member of the first specific binding pair is selected from the group consisting of the first dye and the first enzyme; and/or
(e4b) the element conjugated to the latent reactive moiety of the second signaling conjugate is the member of the second specific binding pair, and wherein the second set of detection reagents further comprises a second member of the second specific binding pair, wherein the second member of the second specific binding pair is selected from the group consisting of the second dye and the second enzyme.

11. The method of claim 9, wherein:
(b1) the first secondary detection reagent comprises a first hapten,
(b2) the first tertiary detection reagent is capable of specifically binding to the first hapten,
(b4b) the first member of the specific binding pair is the first hapten and the second member of the first specific binding pair is the first tertiary detection reagent;
and/or
(e1) the second secondary detection reagent comprises a second hapten,
(e2) the second tertiary detection reagent is capable of specifically binding to the second hapten,
(e4b) the first member of the second specific binding pair is the second hapten and the second member of the second specific binding pair is the second tertiary detection reagent.

12. The method of claim 1, further comprising:
scoring staining in the first portion of the sample and the second portion of the sample; and
determining the presence of the fusion protein based on the scores.

13. The method of claim 12, wherein the first portion of the sample and the second portion of the sample are scored by determining an intensity score, wherein a higher intensity score in the first portion of the sample than in the second portion of the sample indicates the presence of a fusion protein.

14. The method of claim 12, wherein the first portion of the sample and the second portion of the sample are scored by determining an H-score, wherein a higher H-score in the first portion of the sample than in the second portion of the sample indicates the presence of a fusion protein.

15. The method of claim 12, wherein the first portion of the sample and the second portion of the sample are scored by determining an percentage of tumor cells with positive staining, wherein a higher percentage of positively-staining tumor cells in the first portion of the sample than in the second portion of the sample indicates the presence of a fusion protein.

16. The method of claim 1, wherein the wild-type protein is encoded by a gene selected from the group consisting of ROS1, RET, ALK, NTRKA, NTRKB, NTRKC, RAF1, BRAF, PRKCA, PRKCB, and PKN1.

17. The method of claim 16, wherein the gene is ROS1, the first biomarker-specific reagent binds to an epitope disposed in amino acids residues 1926-2347 of SEQ ID NO: 1, and the second biomarker-specific reagent binds to an epitope disposed in amino acids residues of 1-1749 SEQ ID NO: 1.

18. A method of preparing a histological or a cytological sample for detecting expression of an oncogenic fusion protein, the method comprising:
(a) contacting the sample with a first biomarker-specific reagent under conditions sufficient to permit specific binding between the first biomarker-specific reagent and a first target, wherein the first target is one of a retained portion of a wild-type counterpart of the oncogenic fusion protein or a lost portion of the wild-type counterpart of the oncogenic fusion protein;
(b) contacting the sample with a first set of detection reagents under conditions sufficient to deposit the first dye on the sample in proximity to the first biomarker specific reagent bound to the sample;
(c) contacting the sample with a second biomarker-specific reagent under conditions sufficient to permit specific binding between the second biomarker-specific reagent and a second target, wherein the second target is the other of the retained portion of a wild-type counterpart of the oncogenic fusion protein or the lost portion of the wild-type counterpart of the oncogenic fusion protein; and
(d) contacting the sample with a second set of detection reagents under conditions sufficient to deposit the second dye on the sample in proximity to the first biomarker specific reagent bound to the sample, wherein the first dye and the second dye are chosen such that:
(d1) the first and the second dye are distinguishable from one another when co-localized on the sample, or
(d2) the first dye generates a first detectable signal when not co-localized with the second dye, the second dye generates a second detectable signal when not co-localized with the first dye, and the first dye and the second dye generate a third detectable signal when co-localized.

19. A multiplex method of preparing a histological or a cytological sample of a tumor for evaluation of the presence or absence of oncogenic rearrangements of ROS1, the method comprising:
(a) affinity enzymatically staining the sample with a first biomarker specific reagent capable of binding to an N-terminal portion of a wild-type human Ros1 protein and a set of detection reagents adapted to deposit a first dye in proximity to the first biomarker specific reagent when bound to the sample, and
(b) affinity enzymatically staining the sample with a second biomarker specific reagent capable of binding to a C-terminal portion of a wild-type human Ros1 protein and a set of detection reagents adapted to deposit a second dye in proximity to the first biomarker specific reagent when bound to the sample, wherein the first dye and the second dye are distinguishable from one another when co-localized on the sample, or the first dye generates a first detectable signal when not co-localized with the second dye, the second dye generates a second detectable signal when not co-localized with the first dye, and the first dye and the second dye generate a third detectable signal when co-localized.
